(19) United States
(12) Patent Application Publication
(10) Pub. No.: US 2011/0207726 A1
(43) Pub. Date: Aug. 25, 2011

(54) Inhibitors of Human Cathepsin L, Cathepsin B, and Cathepsin S

(75) Inventors: Scott L. Diamond, Bala Cynwyd, PA (US); Mary Pat Beavers, New Hope, PA (US); Donna Huryn, Allenton, NJ (US); Michael C. Myers, East Berlin, PA (US); Amos B. Smith, Merion, PA (US); Parag P. Shah, Philadelphia, PA (US); Zhuqing Liu, Philadelphia, NJ (US)

(73) Assignee: The Trustees of the University of Pennsylvania

(21) Appl. No.: 12/937,495

(22) PCT Filed: Apr. 17, 2009
(86) PCT No.: PCT/US2009/002412

§ 371 Date: May 11, 2011

Related U.S. Application Data
(60) Provisional application No. 61/124,549, filed on Apr. 17, 2008.
(60) Provisional application No. 61/124,521, filed on Apr. 17, 2008.

(51) Int. Cl. A61K 31/538; C07D 209/20; C07D 401/12; C07D 413/12; C07D 403/12; C07D 207/09; A61K 31/4045; A61K 31/4709; A61K 31/4725; A61K 31/40; C12N 9/99; A61P 35/00; A61P 19/10; A61P 19/02; A61P 31/12; A61P 31/14; A61P 17/14; A61P 25/00; A61P 11/06; A61P 17/06

(52) U.S. Cl. 514/230.5; 548/495; 546/165; 544/105; 548/455; 546/146; 548/467; 548/537; 514/419; 514/314; 514/414; 514/307; 514/423; 435/184

(57) ABSTRACT

The present invention is directed to novel protease inhibitors that are specific for cathepsin L, cathepsin B, and cathepsin S. Accordingly, the present invention encompasses compositions and methods for treating and preventing diseases and disorders associated with cathepsin L, cathepsin B, or cathepsin S function or activity.

### BACKGROUND OF THE INVENTION

Cysteine proteases, ubiquitous in nature, are frequent targets of drug discovery efforts due to role they play in a number of physiological and pathophysiological processes. In mammals, three classes of cysteine proteases have been characterized: papain-like (such as the cysteinyl cathepsins), calpains, and caspases (Schirmeister et al., 2003, Mini Rev. Med. Chem. 3:361; Vasiljevera et al., 2007, Curr. Pharm. Des. 13:385). Papain-like cysteine proteases play a role in protein turnover, and their overexpression or disregulation has been implicated in certain inflammatory diseases, in cancer, and in osteoporosis, arthritis, among other diseases. Inappropriate activity of calpains has also been associated with a number of disease conditions including neurodegeneration, muscular dystrophy and diabetes. Caspases play a role in the inflammatory process and in apoptosis; their inhibition has been suggested as an approach to degenerative diseases such as arthritis and stroke. A number of infectious agents (bacteria, viruses, and protozoa) also utilize either host or their own cysteine proteases for infectivity, virulence and/or replication processes, and targeting these proteases has been a popular strategy for anti-infective drug discovery efforts (McKerrow et al., 1999, Bioorg. Med. Chem. 7:639).

Inhibitors of cysteine proteases typically rely on the presence of a “warhead” to provide a site for nucleophilic attack by the active site cysteine thiolate (Hernandez et al., 2002, Curr. Opin. Chem. Biol. 6:459). Examples of warheads include peptidyl halomethyl ketones (Schoellmann et al., 1963, Biochemistry 2:252; Rasnick, 1985, Anal. Biochem. 149:461; Raubet et al., 1986, Biochem. J. 239:633), peptidyl diazomethanes (Crawford et al., 1988, Biochem. J. 253:751), peptide aldehydes (Yasuma et al., 1998, J. Med. Chem. 41:4301), acyloxymethyl ketones (Smith et al., 1988, J. Am. Chem. Soc. 110:4429, epoxysuccinyl derivatives (Fujishima et al., 1997, FEBS Lett. 407:47), epoxyketones (Spaltenstein et al., 1996, Tetrahedron Lett. 37:1343), α-aminoalkyl epoxides (Albeck et al., 1995, Bioorg. Med. Chem. Lett. 5:1767), α-keto-aldehydes (Lynas et al., 2000, Bioorg. Med. Chem. Lett. 10:1771), azepanones (Marquis et al., 2005, J. Med. Chem. 48:6870), aziridines and azodicarboxamides (Schirmeister, 1999, J. Med. Chem. 42:560; Radim et al., 2006, Chem. Med. Chem. 1:1126), vinyl sulfones (Palmer et al., 1995, J. Med. Chem. 38:3193), and aza peptides (Xing et al., 1998, J. Med. Chem. 41:1344).

There remains in the art, however, a need for novel and specific inhibitors of cysteine proteases, including Cathepsin L, Cathepsin B, and Cathepsin S inhibitors. The present invention fills this need.

### SUMMARY OF THE INVENTION

In one embodiment the invention includes a composition comprising at least one compound of Formula I, or any pharmaceutically-acceptable salt thereof:

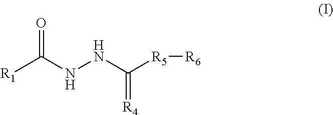

where:
R₁ is —CHR₂R₃ or heterocyclyl;
R₂ is H, —NR₇R₈, —SR₇, acyl, aroyl, heteroaroyl, alkyl, substituted alkyl, aryl, substituted aryl, heteroaryl, substituted heteroaryl, heterocyclyl, or substituted heterocyclyl;
R₃ is H, —CHR₇R₈, alkyl, substituted alkyl, acyl, aroyl, heteroaroyl, aryl, substituted aryl, heteroaryl, substituted heteroaryl, heterocyclyl, substituted heterocyclyl —OR₇, or —SR₇;
R₄ is O or S;
R₅ is —O—, —S—, —NR₇— or a chemical bond;
R₆ is H, alkyl, substituted alkyl, aryl, substituted aryl, heteroaryl, substituted heteroaryl, heterocyclyl, or substituted heterocyclyl; and,
R₇ and R₈ are independently H, aroyl, heteroaroyl, alkyl, substituted alkyl, aryl, substituted aryl, heteroaryl, substituted heteroaryl, heterocyclyl, or substituted heterocyclyl.

In one aspect, R₄ is O and R₅ is —S—. In another aspect, at least one compound of Formula I is selected from the group consisting of Compound No. 1, Compound No. 5, Compound No. 6, Compound No. 7, Compound No. 8, Compound No. 9, Compound No. 10, Compound No. 11, Compound No. 12, Compound No. 13, Compound No. 14, Compound No. 15, Compound No. 16, Compound No. 17, Compound No. 18, Compound No. 19, Compound No. 20, Compound No. 21, Compound No. 22, Compound No. 23, Compound No. 24, Compound No. 25, Compound No. 26, Compound No. 27, Compound No. 28, Compound No. 29, Compound No. 30, Compound No. 31, Compound No. 32, Compound No. 33, Compound No. 34, Compound No. 35, Compound No. 36, Compound No. 37, Compound No. 38, Compound No. 39, Compound No. 40, Compound No. 41, Compound No. 42, Compound No. 43, Compound No. 44, Compound No. 45, Compound No. 46, Compound No. 47, Compound No. 48, Compound No. 49, Compound No. 50, Compound No. 51, Compound No. 52, Compound No. 53, Compound No. 54, Compound No. 55, Compound No. 56, Compound No. 57, Compound No. 58, Compound No. 59, Compound No. 60, Compound No. 61, Compound No. 62, Compound No. 63, Compound No. 64, Compound No. 65, Compound No. 66, Compound No. 67, Compound No. 68, Compound No. 69, Compound No. 70, Compound No. 71, Compound No. 72, Compound No. 73, Compound No. 74, Compound No. 75, Compound No. 76, Compound No. 77, Compound No. 78, Compound No. 79, Compound No. 80, Compound No. 81, Compound No. 82, Compound No. 83, Compound No. 84, Compound No. 85, Compound No. 86, Compound No. 87, Compound No. 88, Compound No. 89, Compound No. 90, Compound No. 91, and Compound No. 92.

In yet another aspect, R₄ is O and R₅ is —O—. In still another aspect, at least one compound of Formula I is selected from the group consisting of Compound No. 93, Compound No. 94, Compound No. 95, Compound No. 96, Compound No. 97, Compound No. 98, Compound No. 99, Compound No. 100, Compound No. 101, Compound No. 102, Compound No. 103, Compound No. 104, Compound No. 105, Compound No. 106, Compound No. 107, Compound No. 108, Compound No. 109, Compound No. 110, Compound No. 111, and Compound No. 112.

In another aspect, R₄ is O and R₅ is a chemical bond. In another aspect, where said at least one compound of Formula I is selected from the group consisting of Compound No. 113, Compound No. 114, Compound No. 115, Compound No. 116, and Compound No. 117.

In another aspect, R₄ is O and R₅ is —NR₇.

In still another aspect, at least one compound of Formula I is Compound No. 125.

In another aspect, R₄ is S and R₅ is —S—.

In yet another aspect, at least one compound of Formula I is Compound No. 126.

In another embodiment the invention includes a pharmaceutical composition comprising a pharmaceutically acceptable carrier, and at least one compound selected from the group consisting of Compound No. 1, Compound No. 5, Compound No. 6, Compound No. 7, Compound No. 8, Compound No. 9, Compound No. 10, Compound No. 11, Compound No. 12, Compound No. 13, Compound No. 14, Compound No. 15, Compound No. 16, Compound No. 17, Compound No. 18, Compound No. 19, Compound No. 20, Compound No. 21, Compound No. 22, Compound No. 23, Compound No. 24, Compound No. 25, Compound No. 26, Compound No. 27, Compound No. 28, Compound No. 29, Compound No. 30, Compound No. 31, Compound No. 32, Compound No. 33, Compound No. 34, Compound No. 35, Compound No. 36, Compound No. 37, Compound No. 38, Compound No. 39, Compound No. 40, Compound No. 41, Compound No. 42, Compound No. 43, Compound No. 44, Compound No. 45, Compound No. 46, Compound No. 47, Compound No. 48, Compound No. 49, Compound No. 50, Compound No. 51, Compound No. 52, Compound No. 53, Compound No. 54, Compound No. 55, Compound No. 56, Compound No. 57, Compound No. 58, Compound No. 59, Compound No. 60, Compound No. 61, Compound No. 62, Compound No. 63, Compound No. 64, Compound No. 65, Compound No. 66, Compound No. 67, Compound No. 68, Compound No. 69, Compound No. 70, Compound No. 71, Compound No. 72, Compound No. 73, Compound No. 74, Compound No. 75, Compound No. 76, Compound No. 77, Compound No. 78, Compound No. 79, Compound No. 80, Compound No. 81, Compound No. 82, Compound No. 83, Compound No. 84, Compound No. 85, Compound No. 86, Compound No. 87, Compound No. 88, Compound No. 89, Compound No. 90, Compound No. 91, Compound No. 92, Compound No. 93, Compound No. 94, Compound No. 95, Compound No. 96, Compound No. 97, Compound No. 98, Compound No. 99, Compound No. 100, Compound No. 101, Compound No. 102, Compound No. 103, Compound No. 104, Compound No. 105, Compound No. 106, Compound No. 107, Compound No. 108, Compound No. 109, Compound No. 110, Compound No. 111, Compound No. 112, Compound No. 113, Compound No. 114, Compound No. 115, Compound No. 116, Compound No. 117, Compound No. 118, Compound No. 119, Compound No. 120, Compound No. 122, Compound No. 123, Compound No. 124, Compound No. 125, Compound No. 126, and Compound No. 127.

In one aspect, at least one compound is selected from the group consisting of Compound No. 1, Compound No. 5, Compound No. 7, Compound No. 8, Compound No. 9, Compound No. 10, Compound No. 11, Compound No. 12, Compound No. 13, Compound No. 14, Compound No. 15, Compound No. 17, Compound No. 18, Compound No. 19, Compound No. 20, Compound No. 21, Compound No. 22, Compound No. 23, Compound No. 24, Compound No. 29, Compound No. 31, Compound No. 34, Compound No. 36, Compound No. 37, Compound No. 38, Compound No. 39, Compound No. 40, Compound No. 41, Compound No. 42, Compound No. 43, Compound No. 44, Compound No. 45, Compound No. 46, Compound No. 47, Compound No. 48, Compound No. 49, Compound No. 50, Compound No. 51, Compound No. 52, Compound No. 53, Compound No. 54, Compound No. 55, Compound No. 56, Compound No. 57, Compound No. 58, Compound No. 62, Compound No. 63, Compound No. 65, Compound No. 66, Compound No. 76, Compound No. 77, Compound No. 78, Compound No. 79, Compound No. 82, Compound No. 83, Compound No. 84, Compound No. 93, Compound No. 94, and Compound No. 96.

In another aspect, at least one compound is selected from the group consisting of Compound No. 7, Compound No. 12, Compound No. 13, Compound No. 18, Compound No. 21, Compound No. 22, Compound No. 23, Compound No. 31, Compound No. 37, Compound No. 38, Compound No. 39, Compound No. 40, Compound No. 41, Compound No. 42, Compound No. 43, Compound No. 44, Compound No. 45, Compound No. 46, Compound No. 47, Compound No. 48, Compound No. 49, Compound No. 50, Compound No. 51, Compound No. 52, Compound No. 53, Compound No. 54, Compound No. 55, Compound No. 56, Compound No. 57, Compound No. 58, Compound No. 62, Compound No. 63, Compound No. 65, Compound No. 66, Compound No. 77, Compound No. 78, Compound No. 79, Compound No. 82, Compound No. 83, Compound No. 84, Compound No. 93, and Compound No. 94.

In another embodiment, the invention includes a method of inhibiting cathepsin L activity, the method comprising contacting a medium comprising cathepsin L with an effective amount of an inhibitor compound selected from the group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof, where when the inhibitor compound contacts the medium comprising cathepsin L, the activity of cathepsin L is inhibited. In one aspect, the thiocarbazate comprises a compound selected from the group consisting of Compound No. 1 and 5-92. In another aspect, the thiocarbazate comprises a compound selected from the group consisting of Compound No. 1, 5, 7, 8-15, 19-24, 29, 31, 34, 36-43, 46, 47, 50-56, 58, 62, 76-78, and 83. In still another aspect, the thiocarbazate comprises a compound selected from the group consisting of Compound No. 7, 12, 13, 21-23, 31, 37-43, 46, 47, 50-56, 58, 62, 76-78, and 83. In another aspect, the oxacarbazate comprises a compound selected from the group consisting of Compound No. 93-112. In still another aspect, the oxacarbazate comprises a compound selected from the group consisting of Compound No. 93, 94, and 96. In another aspect, the oxacarbazate comprises a compound selected from the group consisting of Compound No. 93 and 94. In yet another aspect, the diacyl hydrazine is selected from the group of compounds consisting of Compound No. 113-117. In another aspect, the acyl hydrazine is selected from the group of compounds consisting of Compound No. 118-119. In yet another aspect, the N-hydroxy-amide consists of Compound No. 120. In another aspect, the dialdehyde consists of Compound No. 121. In another aspect, the sulfonylated acyl hydrazine consists of Compound No. 122. In still another aspect, the acyl hydrazone selected from the group of compounds consisting of Compound No. 123-124. In another aspect, the acyl hydrazine carboxamide consists of Compound No. 125. In still another aspect, the acyl hydrazine carbodithioate consists of Compound No. 126. In yet another aspect, the acyl hydrazine oxoacetamide consists of Compound No. 127.

Yet another embodiment of the invention includes a method of inhibiting cathepsin B activity, the method comprising contacting a medium comprising cathepsin B with an effective amount of an inhibitor compound selected from the group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof, where when the inhibitor compound contacts the medium comprising cathepsin B, the activity of the cathepsin B is inhibited. In one aspect, the thiocarbazate comprises a compound selected from the group consisting of Compound No. 1 and 5-92. In another aspect, the oxacarbazate comprises a compound selected from the group consisting of Compound No. 93-112. In still another aspect, the diacyl hydrazine is selected from the group of compounds consisting of Compound. No. 113-117. In yet another aspect, the acyl hydrazine is selected from the group of compounds consisting of Compound No. 118-119. In still another aspect, the N-hydroxy-amide consists of Compound No. 120. In yet another aspect, the dialdehyde consists of Compound No. 121. In another aspect, the sulfonylated acyl hydrazine consists of Compound No. 122. In still another aspect, the acyl hydrazone selected from the group of compounds consisting of Compound No. 123-124. In another aspect, the acyl hydrazine carboxamide consists of Compound No. 125. In yet another aspect, the acyl hydrazine carbodithioate consists of Compound No. 126. In yet another aspect, the acyl hydrazine oxoacetamide consists of Compound No. 127.

Yet another embodiment of the invention includes a method of inhibiting cathepsin S activity, the method comprising contacting a medium comprising cathepsin S with an effective amount of an inhibitor compound selected from the group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof, where when the inhibitor compound contacts the medium comprising cathepsin S, the activity of the cathepsin S is inhibited. In one aspect, the thiocarbazate comprises a compound selected from the group consisting of Compound No. 1 and 5-92. In another aspect, the thiocarbazate comprises a compound selected from the group consisting of Compound No. 13, 14, 17-20, 24, 29, 31, 34, 41, 44-54, 57, 58, 62, 63, 76, 77, and 82-84. In another aspect, the thiocarbazate comprises a compound selected from the group consisting of Compound No. 13, 18, 31, 41, 44-54, 57, 58, 62, 63, and 77. In still another aspect, the oxacarbazate comprises a compound selected from the group consisting of Compound No. 93-112. In yet another aspect, the oxacarbazate comprises a compound selected from the group consisting of Compound No. 93, 94, and 96. In another aspect, the oxacarbazate comprises a compound selected from the group consisting of Compound No. 93 and 94. In still another aspect, the diacyl hydrazine is selected from the group of compounds consisting of Compound No. 113-117. In another aspect, the acyl hydrazine is selected from the group of compounds consisting of Compound No. 118-119. In yet another aspect, the N-hydroxy-amide consists of Compound No. 120. In another aspect, the dialdehyde consists of Compound No. 121. In still another aspect, the sulfonylated acyl hydrazine consists of Compound No. 122. In yet another aspect, the acyl hydrazone selected from the group of compounds consisting of Compound No. 123-124. In still another aspect, the acyl hydrazine carboxamide consists of Compound No. 125. In yet another aspect, the acyl hydrazine carbodithioate consists of Compound No. 126. In another aspect, the acyl hydrazine oxoacetamide consists of Compound No. 127.

Still another embodiment of the invention includes a method of treating a subject infected by or at risk of infection by, a viral pathogen, the method comprising administering a therapeutically effective amount of at least one cathepsin L inhibitor to the subject in need thereof, where the cathepsin L inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one aspect, the cathepsin L inhibitor of the invention comprises a thiocarbazate of Compound No. 1 and 5-92. In another aspect, the thiocarbazate is selected from the group of compounds consisting of Compound No. 1, 5, 7, 8-15, 19-24, 29, 31, 34, 36-43, 46, 47, 50-56, 58, 62, 76-78, and 83. In yet another aspect, the cathepsin L inhibitor comprises an oxacarbazate selected from the group of compounds consisting of Compound No. 93-112. In another aspect, the oxacarbazate selected from the group of compounds consisting of Compound No. 93, 94, and 96. In yet another aspect, the cathepsin L inhibitor comprises a diacyl hydrazine selected from the group of compounds consisting of Compound No. 113-117. In another aspect, the cathepsin L inhibitor comprises an acyl hydrazine selected from the group of compounds consisting of Compound No. 118-119. In still another aspect, the cathepsin L inhibitor comprises an N-hydroxy-amide consisting of Compound No. 120. In another aspect, the cathepsin L inhibitor comprises a sulfonylated acyl hydrazine consisting of Compound No. 122. In still another aspect, the cathepsin L inhibitor comprises an acyl hydrazone selected from the group of compounds consisting of Compound No. 123-124. In another aspect, the cathepsin L inhibitor comprises an acyl hydrazine carboxamide consisting of Compound No. 125. In yet another aspect, the cathepsin L inhibitor comprises an acyl hydrazine carbodithioate consisting of Compound No. 126. In another aspect, the cathepsin L inhibitor comprises an acyl hydrazine oxoacetamide consisting of Compound No. 127. In still another aspect, the infection is selected from the group consisting of SARS, Ebola, and Hendra virus.

Another embodiment of the invention includes a method of treating a subject infected by or at risk of infection by, a viral pathogen, the method comprising administering a therapeutically effective amount of at least one cathepsin B inhibitor to the subject in need thereof, wherein the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one aspect, the cathepsin B inhibitor of the invention comprises a thiocarbazate of Compound No. 1 and 5-92. In another aspect the, cathepsin B inhibitor comprises an oxacarbazate selected from the group of compounds consisting of Compound No. 93-112. In another aspect, the said cathepsin B inhibitor comprises a diacyl hydrazine selected from the group of compounds consisting of Compound No. 113-117. In still another aspect, the cathepsin B inhibitor comprises an acyl hydrazine selected from the group of compounds consisting of Compound No. 118-119. In another aspect, the cathepsin B inhibitor comprises an N-hydroxy-amide consisting of Compound No. 120. In another aspect, the cathepsin B inhibitor comprises a sulfonylated acyl hydrazine consisting of Compound No. 122. In still another aspect, the cathepsin B inhibitor comprises an acyl hydrazone selected from the group of compounds consisting of Compound No. 123-124. In another aspect, the cathepsin B inhibitor comprises an acyl hydrazine carboxamide consisting of Compound No. 125. In yet another aspect, the cathepsin B inhibitor comprises an acyl hydrazine carbodithioate consisting of Compound No. 126. In another aspect, the cathepsin B inhibitor comprises an acyl hydrazine oxoacetamide consisting of Compound No. 127. In yet another aspect, the infection is selected from the group consisting of SARS, Ebola, and Hendra virus.

In another embodiment, the invention includes a method of treating a subject afflicted with cancer, the method comprising the method comprising administering a therapeutically effective amount of at least one cathepsin B inhibitor to the subject in need thereof, wherein the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one aspect, the cathepsin B inhibitor of the invention comprises a thiocarbazate of Compound No. 1 and 5-92. In another aspect the, cathepsin B inhibitor comprises an oxacarbazate selected from the group of compounds consisting of Compound No. 93-112. In another aspect, the said cathepsin B inhibitor comprises a diacyl hydrazine selected from the group of compounds consisting of Compound No. 113-117. In still another aspect, the cathepsin B inhibitor comprises an acyl hydrazine selected from the group of compounds consisting of Compound No. 118-119. In another aspect, the cathepsin B inhibitor comprises an N-hydroxy-amide consisting of Compound No. 120. In another aspect, the cathepsin B inhibitor comprises a sulfonylated acyl hydrazine consisting of Compound No. 122. In still another aspect, the cathepsin B inhibitor comprises an acyl hydrazone selected from the group of compounds consisting of Compound No. 123-124. In another aspect, the cathepsin B inhibitor comprises an acyl hydrazine carboxamide consisting of Compound No. 125. In yet another aspect, the cathepsin B inhibitor comprises an acyl hydrazine carbodithioate consisting of Compound No. 126. In another aspect, the cathepsin B inhibitor comprises an acyl hydrazine oxoacetamide consisting of Compound No. 127. In yet another aspect, the infection is selected from the group consisting of SARS, Ebola, and Hendra virus.

Another embodiment of the invention includes a method of treating a subject afflicted with or at risk of developing osteoporosis, the method comprising the method comprising administering a therapeutically effective amount of at least one cathepsin B inhibitor to the subject in need thereof, wherein the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one aspect, the cathepsin B inhibitor of the invention comprises a thiocarbazate of Compound No. 1 and 5-92. In another aspect the, cathepsin B inhibitor comprises an oxacarbazate selected from the group of compounds consisting of Compound No. 93-112. In another aspect, the said cathepsin B inhibitor comprises a diacyl hydrazine selected from the group of compounds consisting of Compound No. 113-117. In still another aspect, the cathepsin B inhibitor comprises an acyl hydrazine selected from the group of compounds consisting of Compound No. 118-119. In another aspect, the cathepsin B inhibitor comprises an N-hydroxy-amide consisting of Compound No. 120. In another aspect, the cathepsin B inhibitor comprises a sulfonylated acyl hydrazine consisting of Compound No. 122. In still another aspect, the cathepsin B inhibitor comprises an acyl hydrazone selected from the group of compounds consisting of Compound No. 123-124. In another aspect, the cathepsin B inhibitor comprises an acyl hydrazine carboxamide consisting of Compound No. 125. In yet another aspect, the cathepsin B inhibitor comprises an acyl hydrazine carbodithioate consisting of Compound No. 126. In another aspect, the cathepsin B inhibitor comprises an acyl hydrazine oxoacetamide consisting of Compound No. 127. In yet another aspect, the infection is selected from the group consisting of SARS, Ebola, and Hendra virus.

Still another embodiment of the invention includes a method of treating a subject afflicted with or at risk of developing arthritis, the method comprising the method comprising administering a therapeutically effective amount of at least one cathepsin B inhibitor to the subject in need thereof, wherein the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one aspect, the cathepsin B inhibitor of the invention comprises a thiocarbazate of Compound No. 1 and 5-92. In another aspect the, cathepsin B inhibitor comprises an oxacarbazate selected from the group of compounds consisting of Compound No. 93-112. In another aspect, the said cathepsin B inhibitor comprises a diacyl hydrazine selected from the group of compounds consisting of Compound No. 113-117. In still another aspect, the cathepsin B inhibitor comprises an acyl hydrazine selected from the group of compounds consisting of Compound No. 118-119. In another aspect, the cathepsin B inhibitor comprises an N-hydroxy-amide consisting of Compound No. 120. In another aspect, the cathepsin B inhibitor comprises a sulfonylated acyl hydrazine consisting of Compound No. 122. In still another aspect, the cathepsin B inhibitor comprises an acyl hydrazone selected from the group of compounds consisting of Compound No. 123-124. In another aspect, the cathepsin B inhibitor comprises an acyl hydrazine carboxamide consisting of Compound No. 125. In yet another aspect, the cathepsin B inhibitor comprises an acyl hydrazine carbodithioate consisting of Compound No. 126. In another aspect, the cathepsin B inhibitor comprises an acyl hydrazine oxoacetamide consisting of Compound No. 127. In yet another aspect, the infection is selected from the group consisting of SARS, Ebola, and Hendra virus.

Still another embodiment of the invention includes a method of treating a subject infected by or at risk of infection by, a viral pathogen, the method comprising administering a therapeutically effective amount of at least one cathepsin S inhibitor to the subject in need thereof, where the cathepsin S inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof, where when the inhibitor compound contacts the medium comprising cathepsin S, the activity of the cathepsin S is inhibited. In one aspect, the thiocarbazate comprises a compound selected from the group consisting of Compound No. 1 and 5-92. In another aspect, the thiocarbazate comprises a compound selected from the group consisting of Compound No. 13, 14, 17-20, 24, 29, 31, 34, 41, 44-54, 57, 58, 62, 63, 76, 77, and 82-84. In another aspect, the thiocarbazate comprises a compound selected from the group consisting of Compound No. 13, 18, 31, 41, 44-54, 57, 58, 62, 63, and 77. In still another aspect, the oxacarbazate comprises a compound selected from the group consisting of Compound No. 93-112. In yet another aspect, the oxacarbazate comprises a compound selected from the group consisting of Compound No. 93, 94, and 96. In another aspect, the oxacarbazate comprises a compound selected from the group consisting of Compound No. 93 and 94. In still another aspect, the diacyl hydrazine is selected from the group of compounds consisting of Compound No. 113-117. In another aspect, the acyl hydrazine is selected from the group of compounds consisting of Compound No. 118-119. In yet another aspect, the N-hydroxy-amide consists of Compound No. 120. In another aspect, the dialdehyde consists of Compound No. 121. In still another aspect, the sulfonylated acyl hydrazine consists of Compound No. 122. In yet another aspect, the acyl hydrazone selected from the group of compounds consisting of Compound No. 123-124. In still another aspect, the acyl hydrazine carboxamide consists of Compound No. 125. In yet another aspect, the acyl hydrazine carbodithioate consists of Compound No. 126. In another aspect, the acyl hydrazine oxoacetamide consists of Compound No. 127.

In still another embodiment, the invention includes a method of treating a subject afflicted with hair loss, the method comprising administering a therapeutically effective amount of at least one cathepsin L inhibitor to the subject in need thereof, where the cathepsin L inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one aspect, the cathepsin L inhibitor of the invention comprises a thiocarbazate of Compound No. 1 and 5-92. In another aspect, the thiocarbazate is selected from the group of compounds consisting of Compound No. 1, 5, 7, 8-15, 19-24, 29, 31, 34, 36-43, 46, 47, 50-56, 58, 62, 76-78, and 83. In yet another aspect, the cathepsin L inhibitor comprises an oxacarbazate selected from the group of compounds consisting of Compound No. 93-112. In another aspect, the oxacarbazate selected from the group of compounds consisting of Compound No. 93, 94, and 96. In yet another aspect, the cathepsin L inhibitor comprises a diacyl hydrazine selected from the group of compounds consisting of Compound No. 113-117. In another aspect, the cathepsin L inhibitor comprises an acyl hydrazine selected from the group of compounds consisting of Compound No. 118-119. In still another aspect, the cathepsin L inhibitor comprises an N-hydroxy-amide consisting of Compound No. 120. In another aspect, the cathepsin L inhibitor comprises a sulfonylated acyl hydrazine consisting of Compound No. 122. In still another aspect, the cathepsin L inhibitor comprises an acyl hydrazone selected from the group of compounds consisting of Compound No. 123-124. In another aspect, the cathepsin L inhibitor comprises an acyl hydrazine carboxamide consisting of Compound No. 125. In yet another aspect, the cathepsin L inhibitor comprises an acyl hydrazine carbodithioate consisting of Compound No. 126. In another aspect, the cathepsin L inhibitor comprises an acyl hydrazine oxoacetamide consisting of Compound No. 127.

In still another embodiment, the invention includes a method of treating a subject afflicted with an autoimmune disease, the method comprising administering a therapeutically effective amount of at least one cathepsin S inhibitor to a subject in need thereof, where the cathepsin S inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof, where when the inhibitor compound contacts the medium comprising cathepsin S, the activity of the cathepsin S is inhibited. In one aspect, the thiocarbazate comprises a compound selected from the group consisting of Compound No. 1 and 5-92. In another aspect, the thiocarbazate comprises a compound selected from the group consisting of Compound No. 13, 14, 17-20, 24, 29, 31, 34, 41, 44-54, 57, 58, 62, 63, 76, 77, and 82-84. In another aspect, the thiocarbazate comprises a compound selected from the group consisting of Compound No. 13, 18, 31, 41, 44-54, 57, 58, 62, 63, and 77. In still another aspect, the oxacarbazate comprises a compound selected from the group consisting of Compound No. 93-112. In yet another aspect, the oxacarbazate comprises a compound selected from the group consisting of Compound No. 93, 94, and 96. In another aspect, the oxacarbazate comprises a compound selected from the group consisting of Compound No. 93 and 94. In still another aspect, the diacyl hydrazine is selected from the group of compounds consisting of Compound No. 113-117. In another aspect, the acyl hydrazine is selected from the group of compounds consisting of Compound No. 118-119. In yet another aspect, the N-hydroxy-amide consists of Compound No. 120. In another aspect, the dialdehyde consists of Compound No. 121. In still another aspect, the sulfonylated acyl hydrazine consists of Compound No. 122. In yet another aspect, the acyl hydrazone selected from the group of compounds consisting of Compound No. 123-124. In still another aspect, the acyl hydrazine carboxamide consists of Compound No. 125. In yet another aspect, the acyl hydrazine carbodithioate consists of Compound No. 126. In another aspect, the acyl hydrazine oxoacetamide consists of Compound No. 127.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a schematic illustration depicting a thiocarbazate scaffold used to develop the thiocarbazate library.

FIG. 2, comprising FIG. 2A and FIG. 2B, is a series of images depicting protease profiling heatmap of twenty-two thiocarbazates at 10 μM against seventy-five proteases. FIG. 2A depicts a heatmap where thiocarbazates from Table 1 are listed by number across the top of the heatmap and proteases tested are listed on the left. FIG. 2B provides a list of proteases that exhibited no inhibition in the presence of the thiocarbazate tested.

FIG. 3 is a schematic illustration depicting the synthesis of 2,5-disubstituted oxadiazoles.

FIG. 4 is a schematic illustration depicting the conversion of an oxadiazole, Compound (i), to a thiocarbazate, Compound (iii).

FIG. 5 is a schematic illustration depicting an example of the known aza-peptide cathepsin inhibitor, Compound (iv). The kₒₙ is >11,000 M⁻¹s⁻¹, papain (Abeles et al., 1992).

FIG. 6 is a schematic illustration depicting the synthetic procedure to prepare thiocarbazates.

FIG. 7 is a schematic illustration depicting the decomposition products of Compound (iii) in DMSO.

FIG. 8, comprising FIG. 8A through FIG. 8C, is a series of images depicting the structure and activity of oxadiazole Compound (ii) and Compound No. 1. FIG. 8A is a schematic illustration of oxadiazole Compound (ii). FIG. 8B is a schematic illustration of thiocarbazate Compound No. 1, the Boc-protected S-enantiomer of the ring opened by-product of the original high throughput screening (HTS) hit. FIG. 8C is a graph depicting activity of Compound No. 1 against human cathepsin L after pre-incubation with the enzyme target for 0 h (◯), 1 h (Δ), 2 h (□), and 4 h (▴).

FIG. 9, comprising FIG. 9A through FIG. 9C, is a series of graphs depicting the dilution protocol for determination of reversibility. FIG. 9A is a graph depicting cathepsin L at 100-fold its final assay concentration (870 ng/mL) and inhibitor at 10-fold its IC₅₀ after 1 hour preincubation (75 nM) were combined and incubated for 1 hour at room temperature at 2 μL. A rapidly reversible inhibitor should dissociate from the enzyme to restore approximately >90% of enzymatic activity. FIG. 9B is a graph depicting reversibility data for Compound No. 1 after 0 min (◯), 15 min (Δ), 1 hr (□), and 4 hr (▴) preincubation with cathepsin L and upon 100-fold dilution into assay buffer containing ZPhe-Arg-AMC. A full enzyme-substrate reaction without inhibitor () served as a positive control. FIG. 9C is a graph depicting reaction progress curve with 4 hr preincubation of cathepsin L and Compound No. 1.

FIG. 10, comprising FIG. 10A and FIG. 10B, is a series of images depicting a mechanism for binding. FIG. 10A is a schematic illustration depicting single-step mechanism for simple, reversible, slow binding inhibition governed by kinetic constants kₒₙ and k_{off}. FIG. 10B is a graph depicting Kₘ and k_{cat} determination for human cathepsin L enzymatic reaction with 2-Phe-Arg-AMC substrate. Kₘ=0.77 μM and k_{cat}=1.5 s⁻¹.

FIG. 11, comprising FIG. 11A and FIG. 11B, is a series of images depicting the inhibition kinetic model and reaction curves for cathepsin L activity with respect to a given agonist. FIG. 11A depicts ordinary differential equations governing the single-step mechanism of inhibition shown in FIG. 10A. FIG. 11B is a graph depicting reaction progress curves (⋄) shown for 8.7 ng/mL human cathepsin L enzyme and 1 μM Z-Phe-Arg-AMC substrate with varying concentrations of Compound No. 1 inhibitor. They have been fit to a five-parameter inhibition kinetic model using APPSPACK optimization software with a linear least squares objective function.

FIG. 12, comprising FIG. 12A and FIG. 12B, is a series of graphs depicting the IC₅₀ of cathepsin L inhibitor Compound No. 1 against various pathogens. FIG. 12A is a graph depicting the activity of Compound No. 1 against Plasmodium falciparum, IC₅₀=15.4±0.6 μM. FIG. 12B is a graph depicting the activity of Compound No. 1 against Leishmania major, determined to be IC₅₀=12.5±0.6 μM.

FIG. 13, comprising FIG. 13A and FIG. 13B, is a series of images depicting the structure and relation ship of Cathepsin L inhibitor CLIK-148 and its structural interaction with papain. FIG. 13A is a schematic illustration of CLIK-148, a cathepsin L-specific inhibitor (Katunuma et al., 1999, FEBS Lett. 458:6-10; Tsuge et al., 1999, Biochem. Biophys. Res. Comm. 266:411-416). FIG. 13B is an image depicting an overlay of papain/CLIK-148 crystal structure (Icvz.pdb) with independently docked epoxide ring-opened form of CLIK-148 (light gray). The XP Glide score for the top-scoring pose was −9.27 kcal/mol.

FIG. 14, comprising FIG. 14A and FIG. 14B, is a series of images depicting the structure and physical interactions of Compound No. 1. FIG. 14A is an image depicting hydrogen bonding interactions between Compound No. 1 and papain involving catalytic residues Gln19, Cys25, Gly66, Asp158 and Trp177. The distance between the Cys25 sulfur atom and the thiocarbazate carbonyl carbon is 3.287 Å. FIG. 14B is an image depicting an overlay of papain/CLIK-148 with a computational model of Compound No. 1 in papain.

FIG. 15 is a schematic illustration depicting a newly developed oxacarbazate cathepsin L inhibitor, Compound No. 96 of the present invention.

FIG. 16 is a schematic illustration depicting the synthetic procedure for preparation of the oxacarbazate Compound No. 96.

FIG. 17 is a graph depicting the activity of the oxacarbazate Compound No. 96 (concentration in μM) in SARS and Ebola viral entry assays. Compound No. 96 was effective at blocking SARS (IC₅₀=273±49 nM) and Ebola (IC₅₀=193±39 nM) entry into cells, but not Vesicular stomatitis virus (VSV; IC₅₀>10 μM).

### DETAILED DESCRIPTION OF THE INVENTION

The present invention is related to the recent discovery and characterization of selective inhibitors of cysteine proteases. In one embodiment, the cysteine protease inhibitor selectively inhibits the function or activity of Cathepsin L. In another embodiment, the cysteine protease inhibitor selectively inhibits the function or activity of Cathepsin B. In still another embodiment, the cysteine protease inhibitor selectively inhibits the function or activity of Cathepsin S.

The cysteine protease inhibitors provided herein include several chemotypes including thiocarbazates, oxacarbazates, diacyl hydrazines, acyl hydrazines, N-hydroxy-amides, dialdehydes, sulfonylated acyl hydrazines, acyl hydrazones, acyl hydrazine carboxamides, acyl hydrazine carbodithioates, and acyl hydrazine oxoacetamides and well as methods of their synthesis, assays of activity, and use in the treatment of a variety of diseases, disorders and conditions.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used herein, the term “derivative” refers to a small molecule that differs in structure from the reference molecule, but retains the essential properties of the reference molecule. A derivative molecule may also include a salt, an adduct, or other variant of the reference molecule.

“Viral infection” as used herein refers to infection by a viral pathogen wherein there is clinical evidence of the infection based on symptoms or based on the demonstration of the presence of the viral pathogen in a biological sample from the individual.

A “non-viral infection” as used herein refers to infection by a non-viral pathogen, such as bacteria, fungus, or, parasite, wherein there is clinical evidence of the infection based on symptoms or based on the demonstration of the presence of the non-viral pathogen in a biological sample from the individual.

As used herein an “individual” refers to an animal, preferably a mammal, including both non-human mammals and humans, and more preferably, refers to a human.

The phrase “inhibit,” as used herein, means to reduce a molecule, a reaction, an interaction, a gene, an mRNA, and/or a protein's expression, stability, function or activity by a measurable amount or to prevent its expression, stability, function, or activity entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The term “infectivity”, as used herein, describes the ability of an organism to enter, survive and multiply in the host, while the “infectiousness” of a disease indicates the comparative ease with which the disease is transmitted to other hosts.

The term “infection,” as used herein, refers to a detrimental colonization of a host organism by a foreign species, including a bacterium, a virus, a fungus, a protozoan, or a parasite. In an infection, the infecting organism seeks to utilize the host's resources to multiply, usually at the expense of the host. The infecting organism, or pathogen, interferes with the normal functioning of the host. The host's response to infection is mounted by the humoral and cellular components of the host's immune system. An “occult infection” is one which presents no symptoms.

A “pathogen” or “infectious agent,” used synonymously herein, refers to any disease-causing virus, bacteria, fungi, protozoa, or parasite that infects and causes disease in an animal or plant.

The expression “effective amount” when used to describe a therapy administered to an individual suffering from an infection refers to the amount of a compound that results in a therapeutically beneficial effect, such as a reversal, elimination, or reduction in the frequency, severity, and/or duration of the symptoms of the infection.

The phrase “treatment of a viral infection,” or the phrase “treatment of an individual infected with a pathogen, specifically a virus,” as used herein, encompasses alleviating, reducing the frequency, severity, and/or duration of, or eliminating one or more symptoms of the viral infection.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv) and humanized antibodies (Harlow et al., 1998, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). As used herein, a “neutralizing antibody” is an immunoglobulin molecule that binds to and blocks, directly or indirectly, the biological activity of the antigen.

A “medium,” as used herein, refers to a solution, a bodily fluid, a cell, or a tissue, either in vivo or in vitro.

As used herein, the term “alkyl” refers to a branched or unbranched saturated hydrocarbon group of 1 to 24 carbon atoms, such as methyl, ethyl, n-propyl, isopropyl, n-butyl, isobutyl, tert-butyl, pentyl, hexyl, heptyl, octyl, decyl and the like. Preferred alkyl groups herein contain 1 to 6 carbon atoms. Alkyl groups may be optionally substituted with one to three groups selected from the group consisting of acyl, aroyl, heteroaroyl, aminoacyl, N-aryl-aminoacyl, N-heteroaryl-aminoacyl, N-heterocyclyl-aminoacyl, heterocyclyl-acyl, acylamino, alkoxycarbonyl, halo, amino, methoxy, ethoxy, hydroxyl, methylthio, methylsulfonyl, nitro, aryl, heterocyclyl and heteroaryl. The term “aminoacyl” refers to the group —C(═O)—NH₂. The term “acylamino” refers to the group —NHC(═O)—X, wherein “X” is a monovalent group.

As used herein, the term “cycloalkyl” refers to ring-containing alkyl radicals. Examples include cyclohexyl, cyclopentyl, cyclopropyl, cyclopropylmethyl and norbornyl. Cycloalkyl groups may be optionally substituted with one to three groups selected from the group consisting of halo, amino, methoxy, ethoxy, hydroxyl, methylthio, methylsulfonyl, nitro, aryl, heterocyclyl and heteroaryl.

As used herein, the term “aryl,” employed alone or in combination with other terms, means, unless otherwise stated, a carbocyclic aromatic group containing one or more rings (typically one, two or three rings). Multiple rings may be attached together in a pendent manner, such as a biphenyl, or may be fused, such as naphthalene. Examples include, but are not limited to, phenyl, anthracyl and naphthyl. Preferred are phenyl and naphthyl, most preferred is phenyl. Aryl groups may be optionally substituted with one to three groups chosen from halo, amino, methoxy, ethoxy, hydroxyl, methylthio, methylsulfonyl, nitro, aryl, heterocyclyl and heteroaryl.

As used herein, the term “heterocycle”, “heterocyclyl” or “heterocyclic” by itself or as part of another substituent means, unless otherwise stated, an unsubstituted or substituted, stable, mono- or multicyclic heterocyclic ring system consisting of carbon atoms and at least one heteroatom selected from the group consisting of N, O, and S, and wherein the nitrogen and sulfur heteroatoms may be optionally oxidized, and the nitrogen atom may be optionally quaternized. The heterocycle may be attached to the compound of which it is a component, unless otherwise stated, at any heteroatom or carbon atom in the heterocycle that affords a stable structure. Heterocyclic groups may be optionally substituted with one to three groups chosen from halo, amino, methoxy, ethoxy, hydroxyl, methylthio, methylsulfonyl, nitro, aryl, heterocyclyl and heteroaryl.

Examples of non-aromatic heterocycles include monocyclic groups such as: aziridinyl, oxiranyl, thiiranyl, azetidinyl, oxetanyl, thietanyl, pyrrolidinyl, pyrrolinyl, imidazolinyl, pyrazolidinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydropyridinyl, 1,4-dihydropyridinyl, piperazinyl, morpholinyl, thiomorpholinyl, pyranyl, 2,3-dihydropyranyl, tetrahydropyranyl, 1,4-dioxanyl, 1,3-dioxanyl, homopiperazinyl, homopiperidinyl, 1,3-dioxepinyl, 4,7-dihydro-1,3-dioxepinyl and hexamethyleneoxide.

As used herein, the term “heteroaryl” or “heteroaromatic” refers to a heterocycle having aromatic character. A monocyclic heteroaryl group is preferably a 5-, 6-, or 7-membered ring, examples of which are pyrrolyl, furyl, thienyl, pyridyl, pyrimidinyl and pyrazinyl. A polycyclic heteroaryl may comprise multiple aromatic rings or may include one or more partially saturated rings. Heteroaryl groups may be optionally substituted with one to three groups selected from the group consisting of halo, amino, methoxy, ethoxy, hydroxyl, methylthio, methylsulfonyl, nitro, aryl, heterocyclyl and heteroaryl.

Examples of monocyclic heteroaryl groups include, for example, six-membered monocyclic aromatic rings such as, for example, pyridyl, pyrazinyl, pyrimidinyl and pyridazinyl; and five-membered monocyclic aromatic rings such as, for example, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heteroaryl groups containing a partially saturated ring include tetrahydroquinolyl and 2,3-dihydrobenzofuryl.

Examples of polycyclic heteroaryls include indolyl, indolinyl, quinolyl, tetrahydroquinolyl, isoquinolyl, 1,2,3,4-tetrahydroisoquinolyl, cinnolinyl, quinoxalinyl, quinazolinyl, phthalazinyl, 1,8-naphthyridinyl, 1,4-benzodioxanyl, chromene-2-one-yl (coumarinyl), dihydrocoumarin, chromene-4-one-yl, benzofuryl, 1,5-naphthyridinyl, 2,3-dihydrobenzofuryl, 1,2-benzisoxazolyl, benzothienyl, benzoxazolyl, benzothiazolyl, purinyl, benzimidazolyl, benzotriazolyl, thioxanthinyl, benzazepinyl, benzodiazepinyl, carbazolyl, carbolinyl, acridinyl, pyrrolizidinyl and quinolizidinyl.

### DESCRIPTION

The invention is based in part on the discovery of novel cysteine protease inhibitors that are substantially selective for inhibiting the function or activity of cathepsin L, cathepsin B, or cathepsin S.

A skilled artisan will appreciate that inhibiting cathepsin activity can be accomplished using any method known in the art. Examples of methods to inhibit cathepsin activity include, but are not limited to decreasing expression of an endogenous cathepsin gene, decreasing expression of cathepsin mRNA, and inhibiting activity of cathepsin protein. A cathepsin inhibitor may therefore be a compound or composition that decreases expression of a cathepsin gene, a compound or composition that decreases cathepsin mRNA half-life, stability and/or expression, or a compound or composition that inhibits cathepsin protein function. A cathepsin inhibitor may be any type of compound, including but not limited to, a polypeptide, a nucleic acid, an aptamer, a peptidomimetic, and a small molecule, or combinations thereof.

Cathepsin inhibition may be accomplished either directly or indirectly. For example, a cathepsin may be directly inhibited by compounds or compositions that directly interact with cathepsin protein, such as antibodies or soluble cathepsin receptors. Alternatively, cathepsin may be inhibited indirectly by compounds or compositions that inhibit cathepsin receptors, cathepsin downstream effectors, or upstream regulators which up-regulate cathepsin expression.

Decreasing expression of an endogenous cathepsin gene includes providing a specific inhibitor of cathepsin gene expression. Decreasing expression of cathepsin mRNA or cathepsin protein includes decreasing the half-life or stability of cathepsin mRNA or decreasing expression of cathepsin mRNA. Methods of decreasing expression of cathepsin include, but are not limited to, methods that use an siRNA, a microRNA, an antibody, a soluble receptor, an antisense nucleic acid, a ribozyme, an expression vector encoding a transdominant negative mutant, a peptide, a small molecule, other specific inhibitors of cathepsin gene, mRNA, and protein expression, and combinations thereof.

In a preferred embodiment, a cysteine protease inhibitor of the instant invention is a small molecule, including a thiocarbazate, an oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, and an acyl hydrazine oxoacetamide. The invention further includes a derivative of any inhibitor disclosed herein.

### I. Compositions

### Compounds of the Invention Encompassed by Formula I:

In one embodiment, the compounds of the present invention are represented by the Formula I, or any pharmaceutically-acceptable salt thereof:

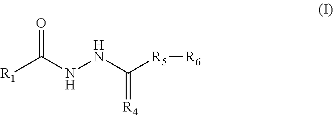

wherein:
R₁ is —CR_{2′}R_{2″}R₃ or heterocyclyl;
R_{2′} and R_{2″} are independently H, —NR₇R₈, —SR₇, acyl, aroyl, heteroaroyl, alkyl, substituted alkyl, aryl, substituted aryl, heteroaryl, substituted heteroaryl, heterocyclyl, or substituted heterocyclyl;
R₃ is H, —CHR₇R₈, alkyl, substituted alkyl, acyl, aroyl, heteroaroyl, aryl, substituted aryl, heteroaryl, substituted heteroaryl, heterocyclyl, substituted heterocyclyl —OR₇, or —SR₇;
R₄ is O or S;
R₅ is —O—, —S—, —C(═O)—, —NR₇— or a chemical bond;
R₆ is H, alkyl, substituted alkyl, aryl, substituted aryl, heteroaryl, substituted heteroaryl, heterocyclyl, or substituted heterocyclyl; and,
R₇ and R₈ are independently H, aroyl, heteroaroyl, alkyl, substituted alkyl, aryl, substituted aryl, heteroaryl, substituted heteroaryl, heterocyclyl, or substituted heterocyclyl.

In one embodiment of the invention, R₄ is O and R₅ is —S—.

In a preferred sub-embodiment, the compounds of the invention are selected from the group consisting of Compound No. 1, Compound No. 5, Compound No. 6, Compound No. 7, Compound No. 8, Compound No. 9, Compound No. 10, Compound No. 11, Compound No. 12, Compound No. 13, Compound No. 14, Compound No. 15, Compound No. 16, Compound No. 17, Compound No. 18, Compound No. 19, Compound No. 20, Compound No. 21, Compound No. 22, Compound No. 23, Compound No. 24, Compound No. 25, Compound No. 26, Compound No. 27, Compound No. 28, Compound No. 29, Compound No. 30, Compound No. 31, Compound No. 32, Compound No. 33, Compound No. 34, Compound No. 35, Compound No. 36, Compound No. 37, Compound No. 38, Compound No. 39, Compound No. 40, Compound No. 41, Compound No. 42, Compound No. 43, Compound No. 44, Compound No. 45, Compound No. 46, Compound No. 47, Compound No. 48, Compound No. 49, Compound No. 50, Compound No. 51, Compound No. 52, Compound No. 53, Compound No. 54, Compound No. 55, Compound No. 56, Compound No. 57, Compound No. 58, Compound No. 59, Compound No. 60, Compound No. 61, Compound No. 62, Compound No. 63, Compound No. 64, Compound No. 65, Compound No. 66, Compound No. 67, Compound No. 68, Compound No. 69, Compound No. 70, Compound No. 71, Compound No. 72, Compound No. 73, Compound No. 74, Compound No. 75, Compound No. 76, Compound No. 77, Compound No. 78, Compound No. 79, Compound No. 80, Compound No. 81, Compound No. 82, Compound No. 83, Compound No. 84, Compound No. 85, Compound No. 86, Compound No. 87, Compound No. 88, Compound No. 89, Compound No. 90, Compound No. 91, and Compound No. 92, or any pharmaceutically-acceptable salt thereof.

In another preferred sub-embodiment, the compounds of the invention are selected from the group consisting of Compound No. 7, Compound No. 12, Compound No. 13, Compound No. 18, Compound No. 21, Compound No. 22, Compound No. 23, Compound No. 25, Compound No. 26, Compound No. 27, Compound No. 28, Compound No. 30, Compound No. 31, Compound No. 33, Compound No. 35, Compound No. 37, Compound No. 38, Compound No. 39, Compound No. 40, Compound No. 41, Compound No. 42, Compound No. 43, Compound No. 44, Compound No. 45, Compound No. 46, Compound No. 47, Compound No. 48, Compound No. 49, Compound No. 50, Compound No. 51, Compound No. 52, Compound No. 53, Compound No. 54, Compound No. 55, Compound No. 56, Compound No. 57, Compound No. 58, Compound No. 59, Compound No. 60, Compound No. 61, Compound No. 62, Compound No. 63, Compound No. 65, Compound No. 66, Compound No. 67, Compound No. 68, Compound No. 70, Compound No. 71, Compound No. 74, Compound No. 75, Compound No. 77, Compound No. 78, Compound No. 79, Compound No. 80, Compound No. 81, Compound No. 82, Compound No. 83, Compound No. 84, Compound No. 85, Compound No. 86, Compound No. 87, Compound No. 88, Compound No. 89, Compound No. 90, Compound No. 91, and Compound No. 92, or any pharmaceutically-acceptable salt thereof.

In another preferred sub-embodiment, the compounds of the invention are selected from the group consisting of Compound No. 1, Compound No. 5, Compound No. 6, Compound No. 8, Compound No. 9, Compound No. 10, Compound No. 11, Compound No. 14, Compound No. 15, Compound No. 16, Compound No. 17, Compound No. 19, Compound No. 20, Compound No. 24, Compound No. 29, Compound No. 32, Compound No. 34, Compound No. 36, Compound No. 64, Compound No. 69, Compound No. 72, Compound No. 73, and Compound No. 76, or any pharmaceutically-acceptable salt thereof.

In another embodiment of the invention, R₄ is O and R₅ is —O—.

In one preferred sub-embodiment, the compounds of the invention are selected from the group consisting of Compound No. 93, Compound No. 94, Compound No. 95, Compound No. 96, Compound No. 97, Compound No. 98, Compound No. 99, Compound No. 100, Compound No. 101, Compound No. 102, Compound No. 103, Compound No. 104, Compound No. 105, Compound No. 106, Compound No. 107, Compound No. 108, Compound No. 109, Compound No. 110, Compound No. 111, and Compound No. 112, or any pharmaceutically-acceptable salt thereof.

In another preferred sub-embodiment, the compounds of the invention are selected from the group consisting of Compound No. 93, Compound No. 94, Compound No. 95, Compound No. 108, Compound No. 111, and Compound No. 112, or any pharmaceutically-acceptable salt thereof.

In yet another preferred sub-embodiment, the compounds of the invention are selected from the group consisting of Compound No. 96, Compound No. 97, Compound No. 98, Compound No. 99, Compound No. 100, Compound No. 101, Compound No. 102, Compound No. 103, Compound No. 104, Compound No. 105, Compound No. 106, Compound No. 107, Compound No. 109, and Compound No. 110, or any pharmaceutically-acceptable salt thereof.

In another embodiment of the invention, R₄ is O and R₅ is a chemical bond.

In one preferred sub-embodiment, the compounds of the invention are selected from the group consisting of Compound No. 113, Compound No. 114, Compound No. 115, Compound No. 116, and Compound No. 117, or any pharmaceutically-acceptable salt thereof.

In yet another preferred sub-embodiment, the compounds of the invention are selected from the group consisting of Compound No. 113, Compound No. 114, Compound No. 116, and Compound No. 117, or any pharmaceutically-acceptable salt thereof.

In yet another preferred sub-embodiment, the compounds of the invention are selected from the group consisting of Compound No. 115, or any pharmaceutically-acceptable salt thereof.

In another embodiment of the invention, R₄ is O and R₅ is —NR₇.

In one preferred sub-embodiment, the compound of the invention is Compound No. 125, or any pharmaceutically-acceptable salt thereof.

In another embodiment of the invention, R₄ is S and R₅ is —S—.

In one preferred embodiment, the compound of the invention is Compound No. 126, or any pharmaceutically-acceptable salt thereof.

### Compounds of the Invention Encompassed by Formula II:

In another embodiment, the compounds of the present invention are represented by the Formula II, or any pharmaceutically-acceptable salt thereof:

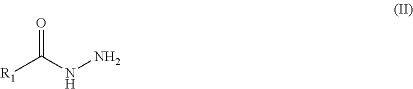

wherein:
R₁ is —CR_{2′}R_{2″}R₃ or heterocyclyl;
R_{2′} and R_{2″} are independently H, —NR₇R₈, —NHC(═O)—O-alkyl, —NHC(═O)—O-aryl, —NHC(═O)—O-heterocyclyl, —SR₇, acyl, aroyl, heteroaroyl, alkyl, substituted alkyl, aryl, substituted aryl, heteroaryl, substituted heteroaryl, heterocyclyl, or substituted heterocyclyl;
R₃ is H, —CHR₇R₈, alkyl, substituted alkyl, acyl, aroyl, heteroaroyl, aryl, substituted aryl, heteroaryl, substituted heteroaryl, heterocyclyl, substituted heterocyclyl —OR₇, or —SR₇. and,
R₇ and R₈ are independently H, aroyl, heteroaroyl, alkyl, substituted alkyl, aryl, substituted aryl, heteroaryl, substituted heteroaryl, heterocyclyl, or substituted heterocyclyl.

In one preferred embodiment, the compounds of the invention are selected from the group consisting of Compound No. 118 and Compound No. 119, or any pharmaceutically-acceptable salt thereof.

### Compounds of the Invention Encompassed by Formula III:

In another embodiment, the compounds of the present invention are represented by the Formula III, or any pharmaceutically-acceptable salt thereof:

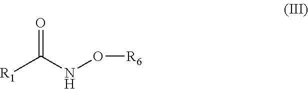

wherein:
R₁ is —CR_{2′}R_{2″}R₃ or heterocyclyl;
R_{2′} and R_{2″} are independently H, —NR₇R₈, —NHC(═O)—O-alkyl, —NHC(═O)—O-aryl, —NHC(═O)—O-heterocyclyl, —SR₇, acyl, aroyl, heteroaroyl, alkyl, substituted alkyl, aryl, substituted aryl, heteroaryl, substituted heteroaryl, heterocyclyl, or substituted heterocyclyl;
R₃ is H, —CHR₇R₈, alkyl, substituted alkyl, acyl, aroyl, heteroaroyl, aryl, substituted aryl, heteroaryl, substituted heteroaryl, heterocyclyl, substituted heterocyclyl —OR₇, or —SR₇;
R₆ is alkyl, substituted alkyl, aryl, substituted aryl, heteroaryl, substituted heteroaryl, heterocyclyl, or substituted heterocyclyl; and,
R₇ and R₈ are independently H, aroyl, heteroaroyl, alkyl, substituted alkyl, aryl, substituted aryl, heteroaryl, substituted heteroaryl, heterocyclyl, or substituted heterocyclyl.

In one preferred embodiment, the compound of the invention is Compound No. 120, or any pharmaceutically-acceptable salt thereof.

### Compounds of the Invention Encompassed by Formula IV:

In one embodiment of the invention, the compounds of the present invention are represented by the Formula IV, or any pharmaceutically-acceptable salt thereof:

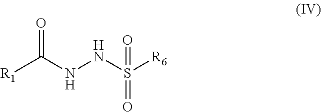

wherein:
R₁ is —CR_{2′}R_{2″}R₃ or heterocyclyl;
R_{2′} and R_{2″} are independently H, —NR₇R₈, —NHC(═O)—O-alkyl, —NHC(═O)—O-aryl, —NHC(═O)—O-heterocyclyl, —SR₇, acyl, aroyl, heteroaroyl, alkyl, substituted alkyl, aryl, substituted aryl, heteroaryl, substituted heteroaryl, heterocyclyl, or substituted heterocyclyl;
R₃ is H, —CHR₇R₈, alkyl, substituted alkyl, acyl, aroyl, heteroaroyl, aryl, substituted aryl, heteroaryl, substituted heteroaryl, heterocyclyl, substituted heterocyclyl —OR₇, or —SR₇;
R₆ is alkyl, substituted alkyl, aryl, substituted aryl, heteroaryl, substituted heteroaryl, heterocyclyl, or substituted heterocyclyl; and
R₇ and R₈ are independently H, aroyl, heteroaroyl, alkyl, substituted alkyl, aryl, substituted aryl, heteroaryl, substituted heteroaryl, heterocyclyl, or substituted heterocyclyl.

In one preferred embodiment, the compound of the invention is Compound No. 122, or any pharmaceutically-acceptable salt thereof.

### Compounds of the Invention Encompassed by Formula V:

In another embodiment, the compounds of the present invention are represented by the Formula V, or any pharmaceutically-acceptable salt thereof:

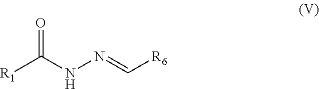

wherein:
R₁ is —CR_{2′}R_{2″}R₃ or heterocyclyl;
R_{2′} and R_{2″} are independently H, —NR₇R₈, —NHC(═O)—O-alkyl, —NHC(═O)—O-aryl, —NHC(═O)—O-heterocyclyl, —SR₇, acyl, aroyl, heteroaroyl, alkyl, substituted alkyl, aryl, substituted aryl, heteroaryl, substituted heteroaryl, heterocyclyl, or substituted heterocyclyl;
R₃ is H, —CHR₇R₈, alkyl, substituted alkyl, acyl, aroyl, heteroaroyl, aryl, substituted aryl, heteroaryl, substituted heteroaryl, heterocyclyl, substituted heterocyclyl —OR₇, or —SR₈;
R₆ is alkyl, substituted alkyl, aryl, substituted aryl, heteroaryl, substituted heteroaryl, heterocyclyl, or substituted heterocyclyl; and
R₇ and R₈ are independently H, aroyl, heteroaroyl, alkyl, substituted alkyl, aryl, substituted aryl, heteroaryl, substituted heteroaryl, heterocyclyl, or substituted heterocyclyl.

In a preferred embodiment, the compound of the invention is selected from the group consisting of Compound No. 123 and Compound No. 124, or any pharmaceutically-acceptable salt thereof.

### Compounds of the Invention Encompassed by Formula VI:

In another embodiment, the compounds of the present invention are represented by the Formula VI, or any pharmaceutically-acceptable salt thereof:

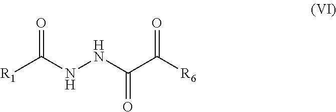

wherein:
R₁ is —CR_{2′}R_{2″}R₃ or heterocyclyl;
R_{2′} and R_{2″} are independently H, —NR₇R₈, —NHC(═O)—O-alkyl, —NHC(═O)—O-aryl, —NHC(═O)—O-heterocyclyl, —SR₇, acyl, aroyl, heteroaroyl, alkyl, substituted alkyl, aryl, substituted aryl, heteroaryl, substituted heteroaryl, heterocyclyl, or substituted heterocyclyl;
R₃ is H, —CHR₇R₈, alkyl, substituted alkyl, acyl, aroyl, heteroaroyl, aryl, substituted aryl, heteroaryl, substituted heteroaryl, heterocyclyl, substituted heterocyclyl —OR₇, or —SR₇;
R₆ is alkyl, substituted alkyl, aryl, substituted aryl, heteroaryl, substituted heteroaryl, heterocyclyl, or substituted heterocyclyl; and
R₇ and R₈ are independently H, aroyl, heteroaroyl, alkyl, substituted alkyl, aryl, substituted aryl, heteroaryl, substituted heteroaryl, heterocyclyl, or substituted heterocyclyl.

In one preferred embodiment, the compound of the invention is Compound No. 127, or any pharmaceutically-acceptable salt thereof.

In one embodiment, the invention includes a cathepsin L inhibitor. A cathepsin L inhibitors comprises a molecule, compound, or agent that inhibits the function activity, or expression of cathepsin L. In a preferred embodiment, the cathepsin L inhibitor comprises a small molecule. In another embodiment, a cathepsin L inhibitor is selected from the group consisting of a thiocarbazate, an oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative thereof. In one embodiment, a cathepsin L inhibitor of the invention is a thiocarbazate selected from the group of compounds consisting of Compound No. 1 and 5-92 (Table 1). In another embodiment, a cathepsin L inhibitor of the invention is a thiocarbazate selected from the group of compounds consisting of Compound No. 1, 5, 7, 8-15, 19-24, 29, 31, 34, 36-43, 46, 47, 50-56, 58, 62, 76-78, and 83 (Table 1). In another embodiment, a cathepsin L inhibitor of the invention is an oxacarbazate selected from the group of compounds consisting of Compound No. 93-112 (Table 2). In another embodiment a cathepsin L inhibitor of the invention is an oxacarbazate selected from the group of compounds consisting of Compound No. 93, 94, and 96 (Table 2). In yet another embodiment, a cathepsin L inhibitor of the invention is a diacyl hydrazine selected from the group of compounds consisting of Compound No. 113-117 (Table 3). In yet another embodiment, a cathepsin L inhibitor of the invention is an acyl hydrazine selected from the group of compounds consisting of Compound No. 118-119 (Table 4). In yet another embodiment, a cathepsin L inhibitor of the invention is an N-hydroxy-amide consisting of Compound No. 120 (Table 5). In yet another embodiment, a cathepsin L inhibitor of the invention is a dialdehyde consisting of Compound No. 121 (Table 6). In yet another embodiment, a cathepsin L inhibitor of the invention is a sulfonylated acyl hydrazine consisting of Compound No. 122 (Table 7). In yet another embodiment, a cathepsin L inhibitor of the invention is a acyl hydrazone selected from the group of compounds consisting of Compound No. 123-124 (Table 8). In yet another embodiment, a cathepsin L inhibitor of the invention is a acyl hydrazine carboxamide consisting of Compound No. 125 (Table 9). In yet another embodiment, a cathepsin L inhibitor of the invention is a acyl hydrazine carbodithioate consisting of Compound No. 126 (Table 10). In yet another embodiment, a cathepsin L inhibitor of the invention is an acyl hydrazine oxoacetamide consisting of Compound No. 127 (Table 11).

In another embodiment, the invention includes a cathepsin B inhibitor. A cathepsin B inhibitor is a molecule, compound, or agent that inhibits the function activity, or expression of cathepsin B. In a preferred embodiment, the cathepsin B inhibitor is a small molecule. In another embodiment, a cathepsin B inhibitor is selected from the group consisting of a thiocarbazate, an oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative thereof. In one embodiment, a cathepsin B inhibitor of the invention is a thiocarbazate selected from the group of compounds consisting of Compound No. 1 and 5-92 (Table 1). In another embodiment, a cathepsin B inhibitor of the invention is a thiocarbazate selected from the group of compounds consisting of Compound No. 21-23, 29, 31, 41, 44, 45, 57, 65, 66, 78 and 79 (Table 1). In another embodiment, a cathepsin B inhibitor of the invention is an oxacarbazate selected from the group of compounds consisting of Compound No. 93-112 (Table 2). In yet another embodiment, a cathepsin B inhibitor of the invention is a diacyl hydrazine selected from the group of compounds consisting of Compound No. 113-117 (Table 3). In yet another embodiment, a cathepsin B inhibitor of the invention is an acyl hydrazine selected from the group of compounds consisting of Compound No. 118-119 (Table 4). In yet another embodiment, a cathepsin B inhibitor of the invention is an N-hydroxy-amide consisting of Compound No. 120 (Table 5). In yet another embodiment, a cathepsin B inhibitor of the invention is a dialdehyde consisting of Compound No. 121 (Table 6). In yet another embodiment, a cathepsin B inhibitor of the invention is a sulfonylated acyl hydrazine consisting of Compound No. 122 (Table 7). In yet another embodiment, a cathepsin B inhibitor of the invention is a acyl hydrazone selected from the group of compounds consisting of Compound No. 123-124 (Table 8). In yet another embodiment, a cathepsin B inhibitor of the invention is a acyl hydrazine carboxamide consisting of Compound No. 125 (Table 9). In yet another embodiment, a cathepsin B inhibitor of the invention is a acyl hydrazine carbodithioate consisting of Compound No. 126 (Table 10). In yet another embodiment, a cathepsin B inhibitor of the invention is an acyl hydrazine oxoacetamide consisting of Compound No. 127 (Table 11).

In still another embodiment, the invention includes a cathepsin S inhibitor. A cathepsin S inhibitor is a molecule, compound, or agent that inhibits the function activity, or expression of cathepsin S. In a preferred embodiment, the cathepsin S inhibitor is a small molecule. In another embodiment, a cathepsin inhibitor is selected from the group consisting of a thiocarbazate, an oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative thereof. In one embodiment, a cathepsin S inhibitor of the invention is a thiocarbazate selected from the group of compounds consisting of Compound No. 1 and 5-92 (Table 1). In another embodiment, a cathepsin S inhibitor of the invention is a thiocarbazate selected from the group of compounds consisting of Compound No. 13, 14, 17-20, 24, 29, 31, 34, 41, 44-54, 57, 58, 62, 63, 76, 77, and 82-84 (Table 1). In another embodiment, a cathepsin S inhibitor of the invention is an oxacarbazate selected from the group of compounds consisting of Compound No. 93-112 (Table 2). In another embodiment a cathepsin S inhibitor of the invention is an oxacarbazate selected from the group of compounds consisting of Compound No. 93, and 96 (Table 2). In yet another embodiment, a cathepsin S inhibitor of the invention is a diacyl hydrazine selected from the group of compounds consisting of Compound No. 113-117 (Table 3). In yet another embodiment, a cathepsin S inhibitor of the invention is an acyl hydrazine selected from the group of compounds consisting of Compound No. 118-119 (Table 4). In yet another embodiment, a cathepsin L inhibitor of the invention is an N-hydroxy-amide consisting of Compound No. 120 (Table 5). In yet another embodiment, a cathepsin S inhibitor of the invention is a dialdehyde consisting of Compound No. 121 (Table 6). In yet another embodiment, a cathepsin S inhibitor of the invention is a sulfonylated acyl hydrazine consisting of Compound No. 122 (Table 7). In yet another embodiment, a cathepsin S inhibitor of the invention is a acyl hydrazone selected from the group of compounds consisting of Compound No. 123-124 (Table 8). In yet another embodiment, a cathepsin S inhibitor of the invention is a acyl hydrazine carboxamide consisting of Compound No. 125 (Table 9). In yet another embodiment, a cathepsin S inhibitor of the invention is a acyl hydrazine carbodithioate consisting of Compound No. 126 (Table 10). In yet another embodiment, a cathepsin S inhibitor of the invention is a acyl hydrazine oxoacetamide consisting of Compound No. 127 (Table 11).

The invention further includes a derivative of any inhibitor disclosed herein.

### A. Chemotypes

### 1. Thiocarbazates

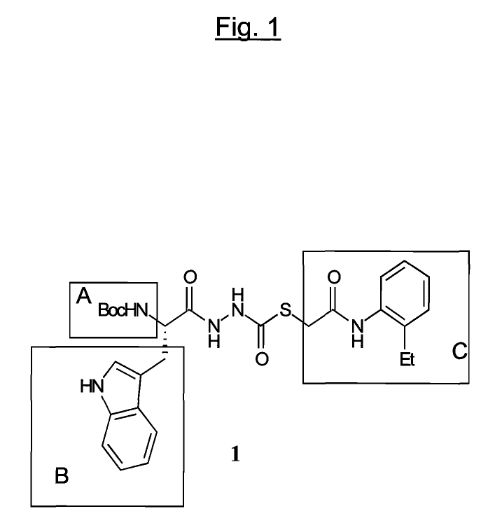

In one embodiment of the present invention, the cysteine protease inhibitor is a thiocarbazate based on a thiocarbazate platform depicted in FIG. 1. Thiocarbazates included in the invention include all compounds listed in Table 1 and designated herein as Compound No. 1, 5-92. Methods of synthesizing the thiocarbazates and other members of the same structural class are disclosed in the Experimental Section of this application. Accordingly, the compounds listed in Table 1 are henceforth called thiocarbazates, herein.

### 2. Oxacarbazates

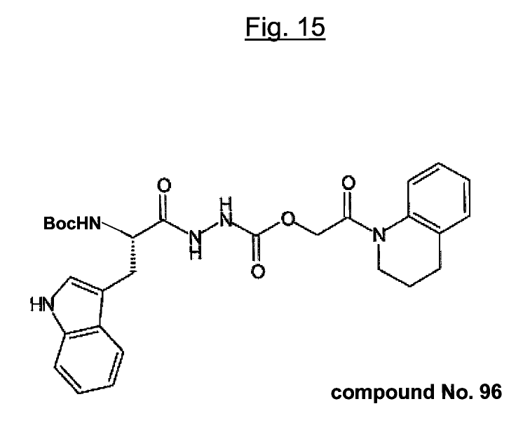

In one embodiment of the present invention, the cysteine protease inhibitor is an oxacarbazate based on the structure depicted in FIG. 15. Oxacarbazates included in the invention include all those compounds depicted in Table 2, and designated herein as Compound No. 93-112. Methods of synthesizing the oxacarbazates and other members of the same structural class are disclosed in the Experimental Section of this application. Accordingly, the compounds listed in Table 2 are henceforth called oxacarbazates, herein.

### 3. Diacyl Hydrazine

In one embodiment of the present invention, the cysteine protease inhibitor is a diacyl hydrazine. Diacyl hydrazones included in the invention include all those compounds depicted in Table 3 and designated herein as Compound No. 113-117. Methods of synthesizing the diacyl hydrazines and other members of the same structural class are disclosed in the Experimental Section of this application. Accordingly, the compounds listed in Table 3 are henceforth called diacyl hydrazines, herein.

### 4. Acyl Hydrazine

In one embodiment of the present invention, the cysteine protease inhibitor is a acyl hydrazine. Acyl hydrazines included in the invention include all those compounds depicted in Table 4 and designated herein as Compound No. 118-119. Accordingly, the compound listed in Table 4 is henceforth called an acyl hydrazine, herein.

### 5. N-Hydroxy-Amide

In one embodiment of the present invention, the cysteine protease inhibitor is an N-hydroxy-amide. N-hydroxy-amides included in the invention includes the compound depicted in Table 5 designated herein as Compound No. 120. Accordingly, the compound listed in Table 5 is henceforth called an N-hydroxy-amide, herein.

### 6. Dialdehyde

In one embodiment of the present invention, the cysteine protease inhibitor is a dialdehyde. Dialdehydes included in the invention include the compound depicted in Table 6 and designated herein as Compound No. 121. Accordingly, the compound listed in Table 6 is henceforth called a dialdehyde, herein.

### 7. Sulfonylated Acyl Hydrazine

In one embodiment of the present invention, the cysteine protease inhibitor is a sulfonylated acyl hydrazine. Sulfonylated acyl hydrazines included in the invention include the compound depicted in Table 7 and designated herein as Compound No. 122. Accordingly, the compound listed in Table 7 is henceforth called a sulfonylated acyl hydrazine, herein.

### 8. Acyl Hydrazone

In one embodiment of the present invention, the cysteine protease inhibitor is an acyl hydrazone. Acyl hydrazones included in the invention include all those compounds depicted in Table 8 and designated herein as Compound No. 123-124. Accordingly, the compounds listed in Table 8 are henceforth called acyl hydrazones, herein.

### 9. Acyl Hydrazine Carboxamide

In one embodiment of the present invention, the cysteine protease inhibitor is an acyl hydrazine carboxamide. Acyl hydrazine carboxamide included in the invention include the compound depicted in Table 9 and designated herein as Compound No. 125. Accordingly, the compound listed in Table 9 is henceforth called an acyl hydrazine carboxamide, herein.

### 10. Acyl Hydrazine Carbodithioate

In one embodiment of the present invention, the cysteine protease inhibitor is an acyl hydrazine carbodithioate. Acyl hydrazine carbodithioate included in the invention includes the compound depicted in Table 10 and designated herein as Compound No. 126. Accordingly, the compound listed in Table 10 is henceforth called an acyl hydrazine carbodithioate, herein.

### 11. Acyl Hydrazone Oxoacetamide

In one embodiment of the present invention, the cysteine protease inhibitor is an acyl hydrazone oxoacetamide. Acyl hydrazone oxoacetamide included in the invention includes the compound depicted in Table 11 and designated herein as Compound No. 127. Accordingly, the compound listed in Table 110 is henceforth called an acyl hydrazone oxoacetamide, herein.

TABLE 1
Thiocarbazates synthesized and assayed against cathepsins B, L, and S.

| Compound | | | IC₅₀ (μM) | | |
|---|---|---|---|---|---|
| No. | Structure | HRMS | Cat B | Cat L | Cat S |
| 1 | 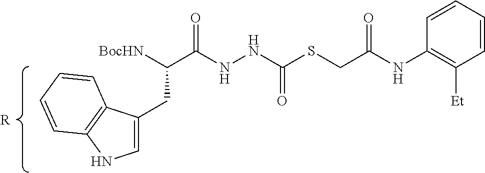 | [M + Na]⁺ 562.2126 | 4.45 | 0.056 | 0.96 |
| 5 | 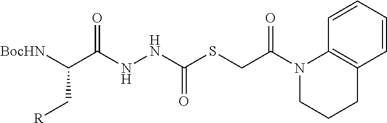 | [M + Na]⁺ 574.2102 | 4.46 | 0.096 | 0.93 |
| 6 | 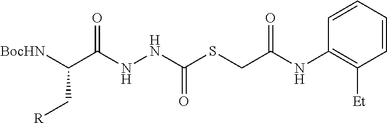 | [M + H]⁺ 487.2383 | >25.0 | >25.0 | >25.0 |
| 7 | 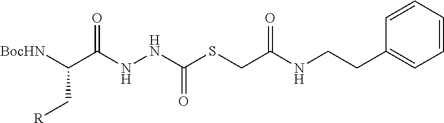 | [M + Na]⁺ 562.2094 | 6.59 | 0.029 | 0.32 |
| 8 | 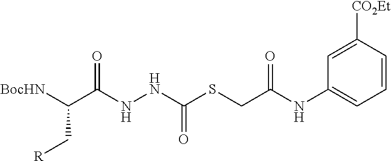 | [M + Na]⁺ 606.1995 | >25.0 | 0.062 | 0.68 |
| 9 | 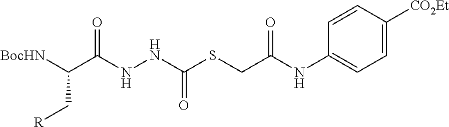 | [M + Na]⁺ 606.1995 | >25.0 | 0.057 | 0.58 |
| 10 | 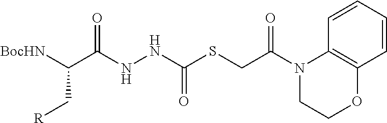 | [M + Na]⁺ 576.1874 | 3.23 | 0.22 | 0.95 |
| 11 | 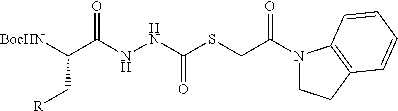 | [M + Na]⁺ 590.1843 | 2.60 | 0.11 | 1.65 |
| 12 | 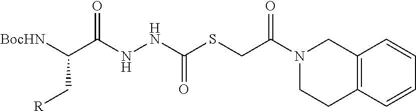 | [M + Na]⁺ 574.2109 | 3.25 | 0.019 | 0.53 |
| 13 | 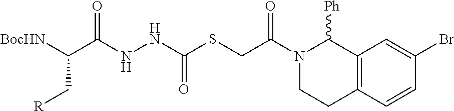 | [M + Na]⁺ 728.1520 | >25.0 | 1.03 | 2.90 |
| 14 | 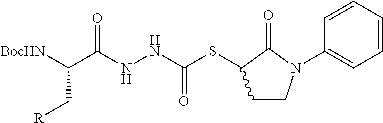 | [M + Na]⁺ 560.1924 | 14.04 | 1.05 | 2.07 |
| 15 | 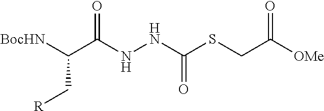 | [M + Na]⁺ 473.1458 | 6.33 | 0.098 | 0.57 |
| 16 | 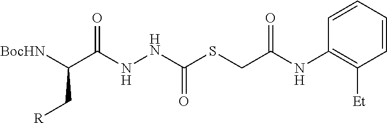 | [M + Na]⁺ 562.2126 | >25.0 | >25.0 | >25.0 |
| 17 | 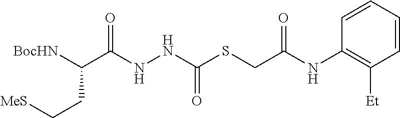 | [M + Na]⁺ 507.1713 | 2.03 | 1.29 | 0.69 |
| 18 | 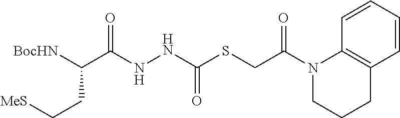 | [M + Na]⁺ 519.1699 | 2.21 | 1.34 | 0.63 |
| 19 | 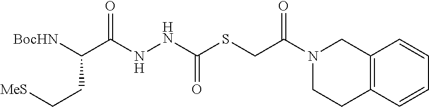 | [M + Na]⁺ 519.1709 | 2.31 | 0.52 | 0.41 |
| 20 | 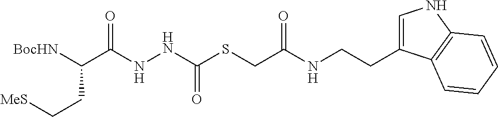 | [M + Na]⁺ 546.1807 | 2.42 | 0.33 | 0.31 |
| 21 | 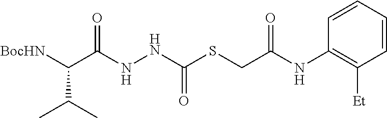 | [M + Na]⁺ 475.2003 | 0.87 | 0.66 | 3.74 |
| 22 | 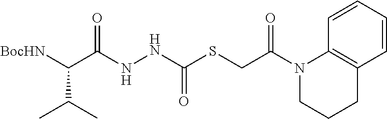 | [M + Na]⁺ 487.1994 | 0.63 | 0.68 | 4.24 |
| 23 | 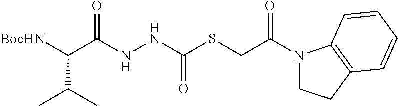 | [M + Na]⁺ 473.1838 | 0.44 | 0.45 | 7.24 |
| 24 | 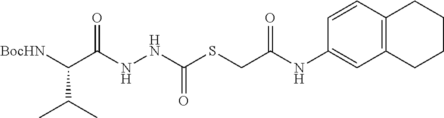 | [M + Na]⁺ 501.2155 | >25.0 | 0.11 | 0.62 |
| 25 | 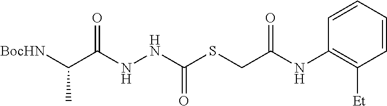 | [M + Na]⁺ 447.1685 | >25.0 | 25.0 | >25.0 |
| 26 | 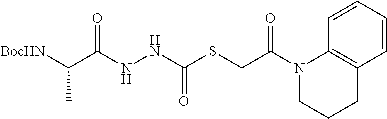 | [M + Na]⁺ 459.1674 | >25.0 | 25.0 | >25.0 |
| 27 | 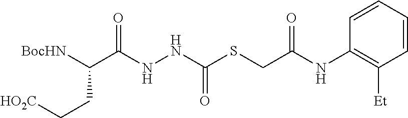 | [M + Na]⁺ 505.1733 | >25.0 | 25.0 | >25.0 |
| 28 | 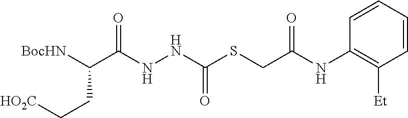 | [M + Na]⁺ 517.1725 | >25.0 | 25.0 | >25.0 |
| 29 | 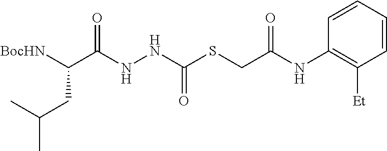 | [M + H]⁺ 467.2318 | 2.63 | 1.09 | 1.65 |
| 30 | 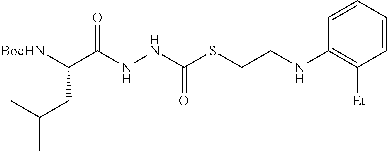 | [M + H]⁺ 453.2516 | >25.0 | >25.0 | >25.0 |
| 31 | 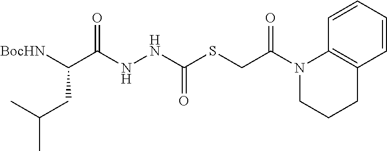 | [M + H]⁺ 479.2327 | 2.72 | 1.64 | 1.64 |
| 32 | 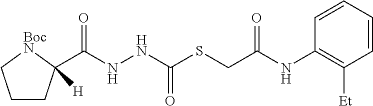 | [M + Na]⁺ 473.1819 | >25.0 | >25.0 | >25.0 |
| 33 | 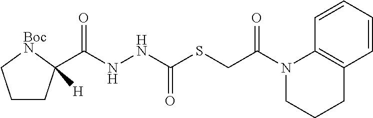 | [M + H]⁺ 463.2014 | >25.0 | >25.0 | >25.0 |
| 34 | 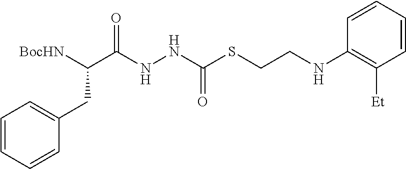 | [M + Na]⁺ 523.1977 | 3.06 | 0.12 | 0.93 |
| 35 | 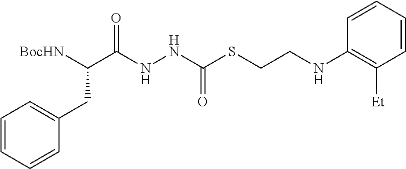 | [M + H]⁺ 487.2383 | >25.0 | >25.0 | >25.0 |
| 36 | 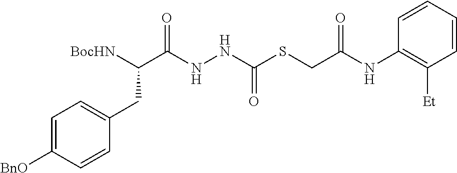 | [M + Na]⁺ 629.2419 | 5.08 | 0.83 | 2.83 |
| 37 | 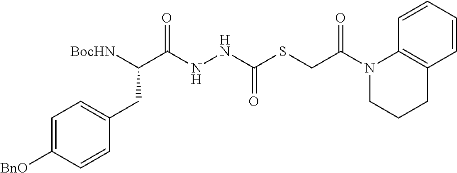 | [M + Na]⁺ 619.2906 | 3.94 | 0.63 | 3.34 |
| 38 | 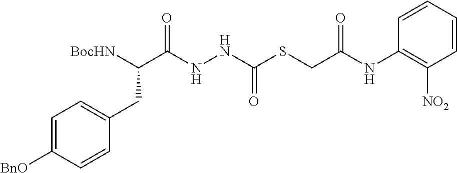 | [M + Na]⁺ 646.1946 | 2.77 | 0.59 | 2.08 |
| 39 | 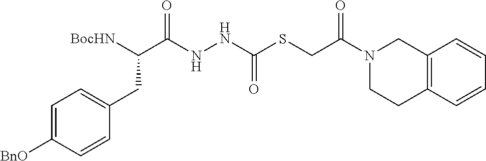 | [M + Na]⁺ 641.2401 | 3.19 | 0.086 | 1.31 |
| 40 | 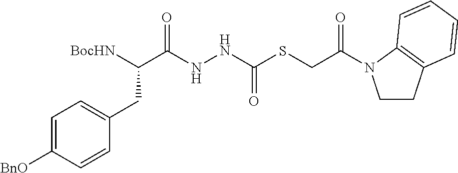 | [M + Na]⁺ 627.2245 | 3.12 | 0.70 | 8.00 |
| 41 | 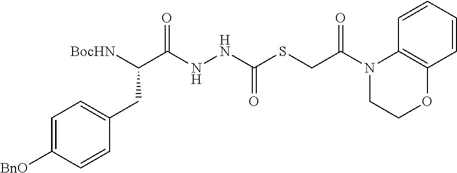 | [M + H]⁺ 643.2224 | 3.03 | 1.56 | 3.98 |
| 42 | 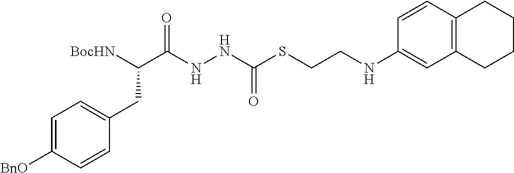 | [M + H]⁺ 655.2554 | >25.0 | 0.50 | 1.90 |
| 43 | 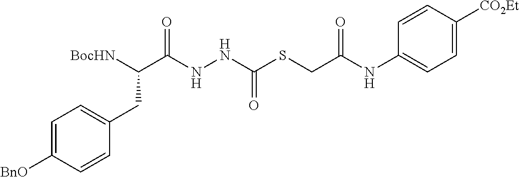 | [M + H]⁺ 673.2322 | >25.0 | 0.14 | 1.08 |
| 44 | 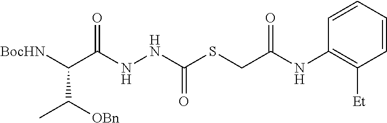 | [M + Na]⁺ 567.2239 | 3.67 | 13.7 | 3.32 |
| 45 | 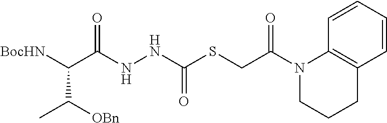 | [M + H]⁺ 579.2236 | 4.35 | >25.0 | 5.15 |
| 46 | 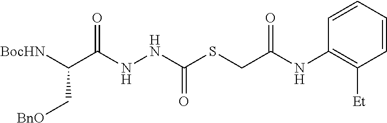 | [M + Na]⁺ 553.2101 | 14.3 | 1.27 | 2.15 |
| 47 | 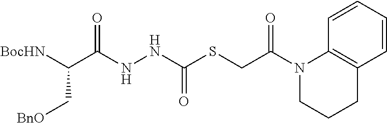 | [M + H]⁺ 565.2097 | 13.2 | 1.51 | 2.64 |
| 48 | 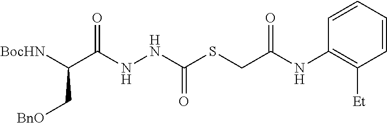 | [M + Na]⁺ 553.2087 | >25.0 | 15.4 | 4.42 |
| 49 | 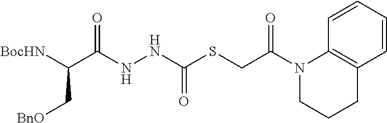 | [M + Na]⁺ 565.2088 | >25.0 | 17.9 | 5.89 |
| 50 | 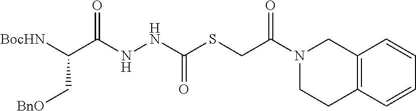 | [M + Na]⁺ 565.2083 | 9.28 | 0.37 | 0.95 |
| 51 | 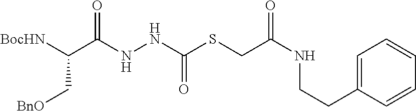 | [M + Na]⁺ 553.2082 | >25.0 | 0.51 | 0.65 |
| 52 | 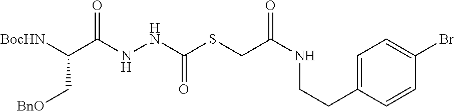 | [M + Na]⁺ 609.1385 | >25.0 | 0.58 | 0.91 |
| 53 | 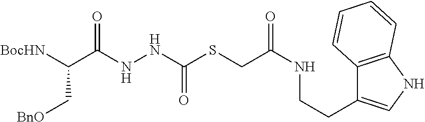 | [M + Na]⁺ 592.2198 | >25.0 | 0.38 | 0.92 |
| 54 | 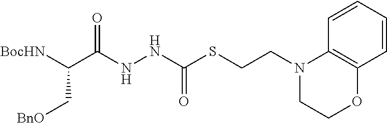 | [M + Na]⁺ 567.1900 | 13.7 | 1.85 | 3.00 |
| 55 | 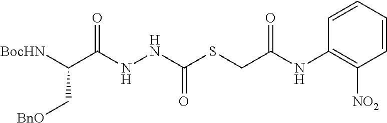 | [M + Na]⁺ 570.1649 | 5.07 | 0.70 | 1.44 |
| 56 | 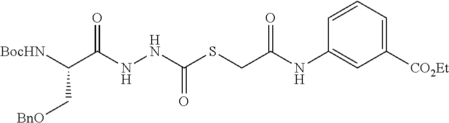 | [M + Na]⁺ 597.1998 | >25.0 | 0.43 | 1.64 |
| 57 | 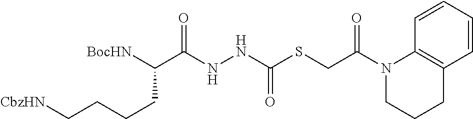 | [M + Na]⁺ 628.286 | 4.35 | >25.0 | 5.15 |
| 58 | 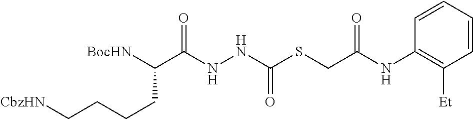 | [M + Na]⁺ 616.2802 | 14.3 | 1.27 | 2.15 |
| 59 | 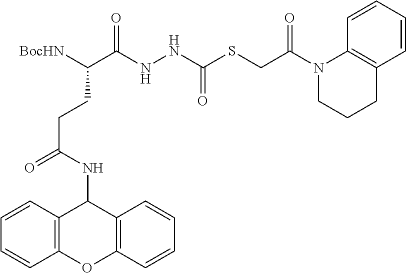 | [M + H]⁺ 662.2664 | >25.0 | >25.0 | >25.0 |
| 60 | 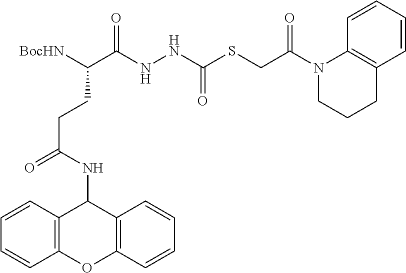 | [M + Na]⁺ 674.2639 | >25.0 | >25.0 | >25.0 |
| 61 | 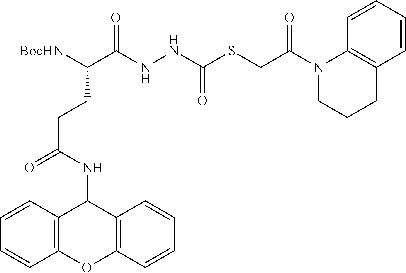 | [M + Na]⁺ 662.2669 | >25.0 | >25.0 | >25.0 |
| 62 | 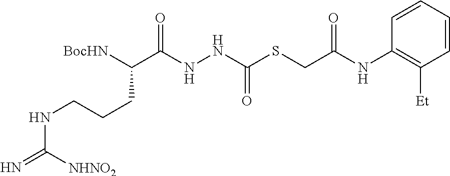 | [M + Na]⁺ 577.2145 | >25.0 | 3.67 | 2.15 |
| 63 | 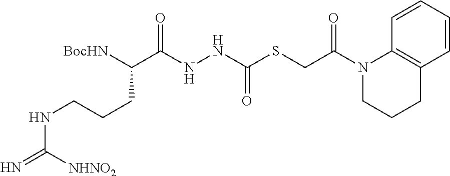 | [M + Na]⁺ 565.2214 | 19.7 | >25.0 | 3.07 |
| 64 | 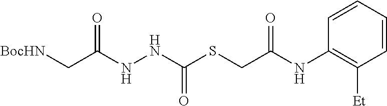 | [M + Na]⁺ 433.1513 | 25.0 | >25.0 | >25.0 |
| 65 | 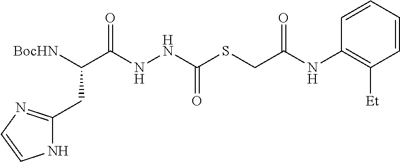 | [M + H]⁺ 491.2057 | 13.7 | >25.0 | >25.0 |
| 66 | 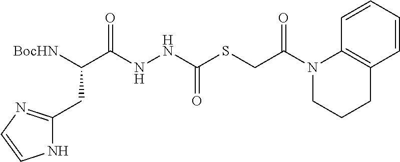 | [M + H]⁺ 503.2071 | 11.6 | >25.0 | >25.0 |
| 67 | 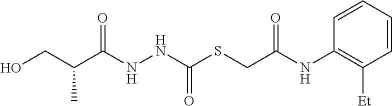 | [M + Na]⁺ 362.1144 | >25.0 | >25.0 | >25.0 |
| 68 | 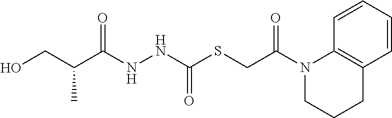 | [M + H]⁺ 374.1152 | >25.0 | >25.0 | >25.0 |
| 69 | 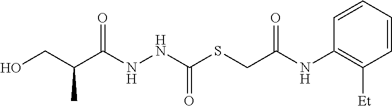 | [M + Na]⁺ 362.1144 | >25.0 | >25.0 | >25.0 |
| 70 | 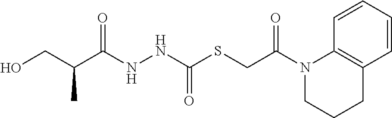 | [M + Na]⁺ 374.1152 | >25.0 | >25.0 | >25.0 |
| 71 | 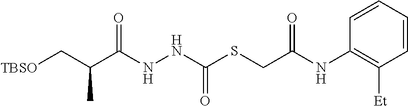 | [M + Na]⁺ 476.2003 | >25.0 | >25.0 | >25.0 |
| 72 | 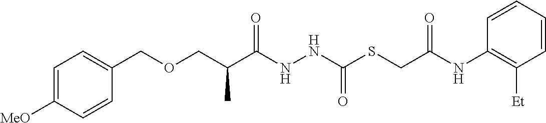 | [M + Na]⁺ 482.1727 | >25.0 | >25.0 | >25.0 |
| 73 | 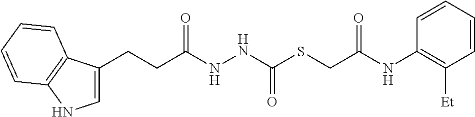 | [M + H]⁺ 447.1485 | >25.0 | >25.0 | >25.0 |
| 74 | 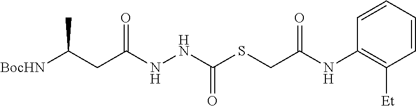 | [M + H]⁺ 439.2004 | >25.0 | >25.0 | >25.0 |
| 75 | 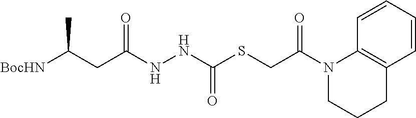 | [M + H]⁺ 451.2012 | >25.0. | >25.0 | >25.0 |
| 76 | 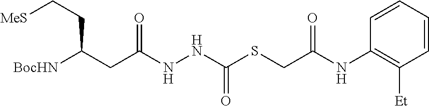 | [M + H]⁺ 499.2047 | >25.0 | 5.52 | 2.41 |
| 77 | 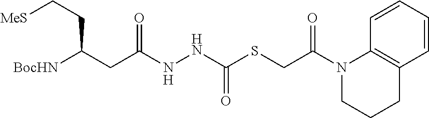 | [M + H]⁺ 511.2044 | >25.0 | 6.95 | 3.01 |
| 78 | 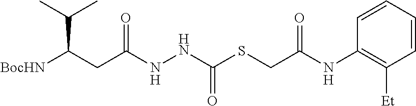 | [M + Na]⁺ 489.2125 | 11.5 | 7.52 | >25.0 |
| 79 | 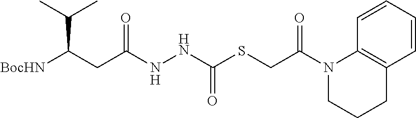 | [M + H]⁺ 479.2319 | 15.8 | >25.0 | >25.0 |
| 80 | 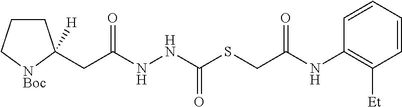 | [M + H]⁺ 465.2172 | >25.0 | >25.0 | >25.0 |
| 81 | 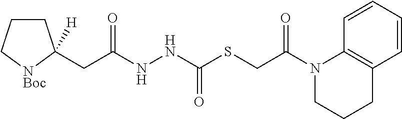 | [M + H]⁺ 477.2177 | >25.0 | >25.0 | >25.0 |
| 82 | 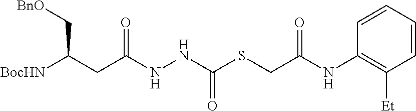 | [M + Na]⁺ 567.2276 | >25.0 | >25.0 | 4.47 |
| 83 | 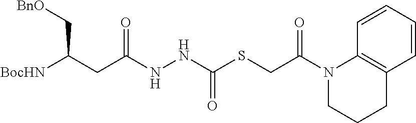 | [M + H]⁺ 557.2435 | >25.0 | 2.12 | 5.95 |
| 84 | 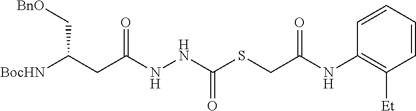 | [M + H]⁺ 545.2438 | >25.0 | >25.0 | 15.2 |
| 85 | 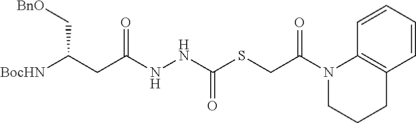 | [M + H]⁺ 557.2454 | >25.0 | >25.0 | >25.0 |
| 86 | 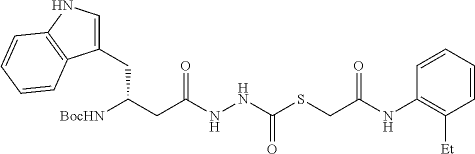 | [M + Na]⁺ 576.2249 | >25.0 | >25.0 | >25.0 |

| Compound | |
|---|---|
| No. | Structure |
| 87 | 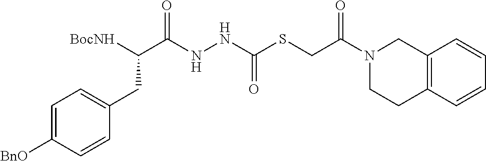 |
| 88 | 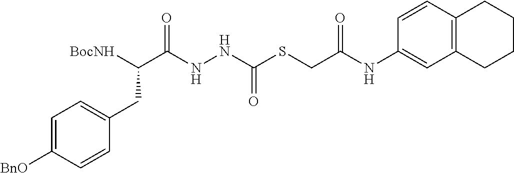 |
| 89 | 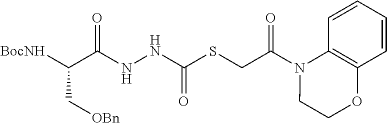 |
| 90 | 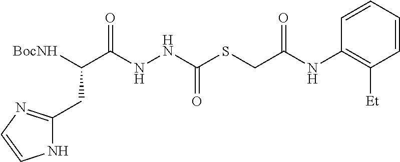 |
| 91 | 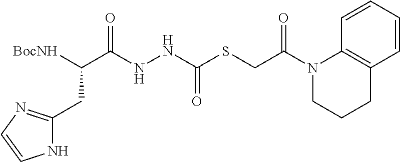 |
| 92 | 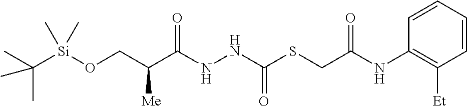 |

TABLE 2
Oxacarbazates.

| Com- | | | | | | |
|---|---|---|---|---|---|---|
| pound | | | Purity | Cat L | Cat B | Cat S |
| No. | Structure | MW | (%) | (μM) | (μM) | (μM) |
| 93 | 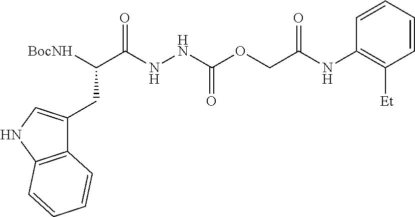 | 523.58 | >95 | 0.011 | 25.000 | 0.062 |
| 94 | 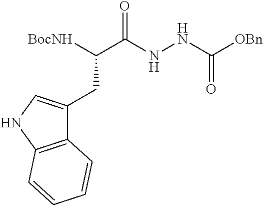 | 452.5 | 95 | 0.806 | 25.000 | 4.105 |
| 95 | 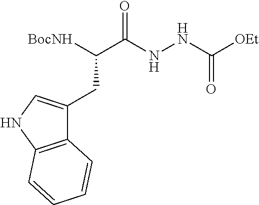 | 390.43 | >95 | 25.000 | 25.000 | 25.000 |
| 96 | 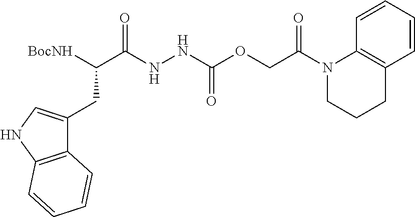 | 535.59 | >95 | 0.006 | 10.128 | 0.025 |
| 97 | 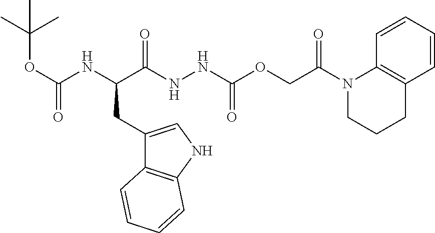 | 535.5 | | | | |
| 98 | 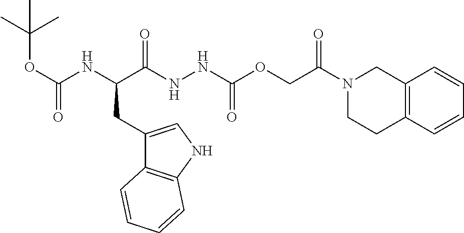 | 535.5 | | | | |
| 99 | 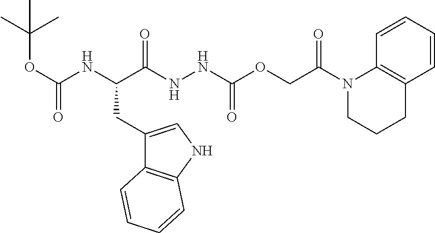 | 535.5 | | | | |
| 100 | 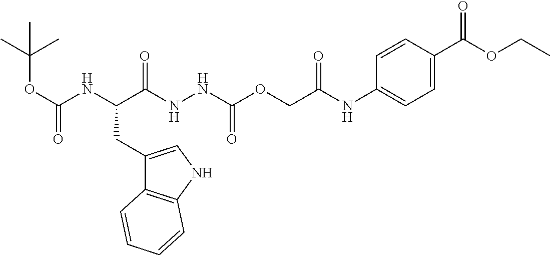 | 567.6 | | | | |
| 101 | 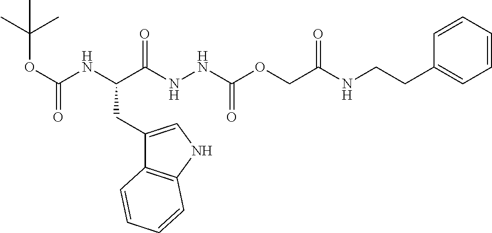 | 523.6 | | | | |
| 102 | 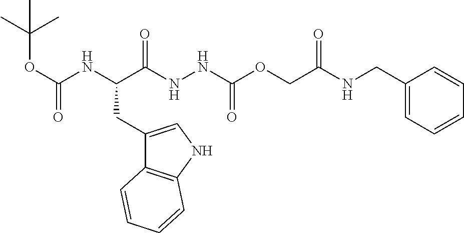 | 509.6 | | | | |
| 103 | 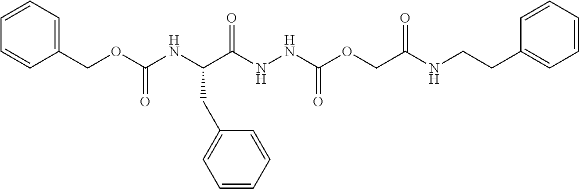 | 518.6 | | | | |
| 104 | 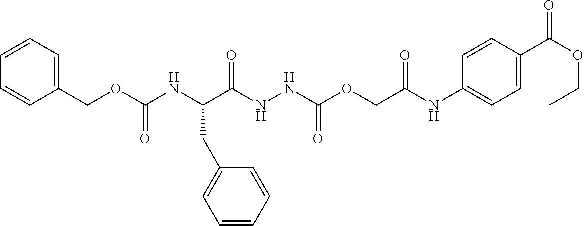 | 562.6 | | | | |
| 105 | 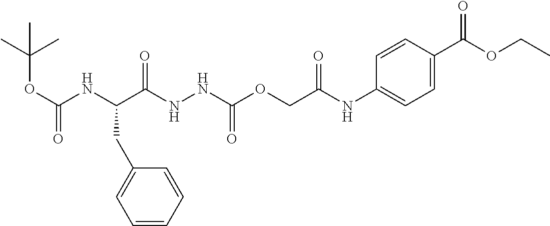 | 528.6 | | | | |
| 106 | 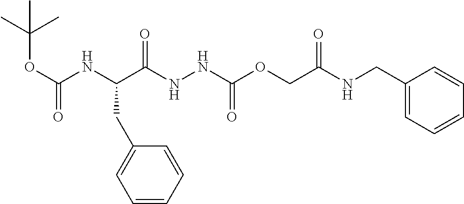 | 528.6 | | | | |
| 107 | 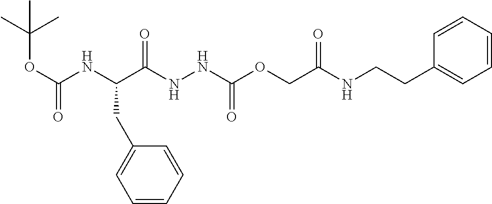 | 484.5 | | | | |
| 108 | 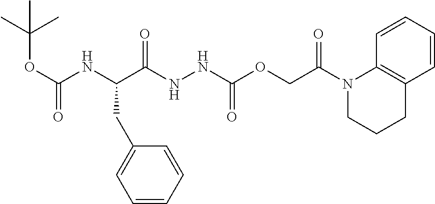 | 496.6 | | | | |
| 109 | 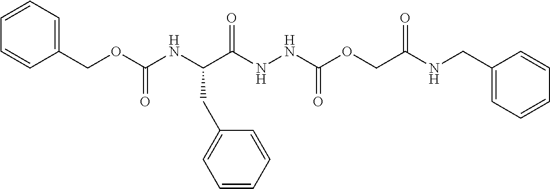 | 504.5 | | | | |
| 110 | 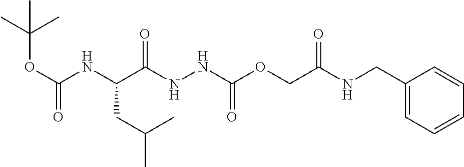 | 436.5 | | | | |
| 111 | 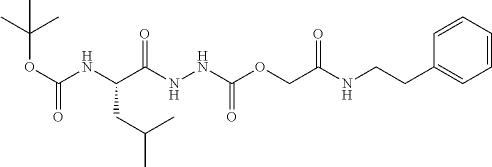 | 450.5 | | | | |
| 112 | 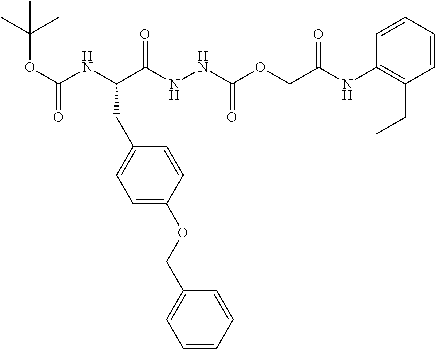 | 590.7 | | | | |

TABLE 3
Diacyl hydrazines.

| Compound | | | Purity | Cat L | Cat B | Cat S |
|---|---|---|---|---|---|---|
| No. | Structure | MW | (%) | (μM) | (μM) | (μM) |
| 113 | 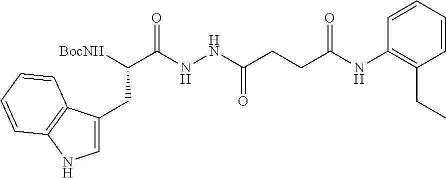 | 521.61 | 99 | 25.000 | 25.000 | 25.000 |
| 114 | 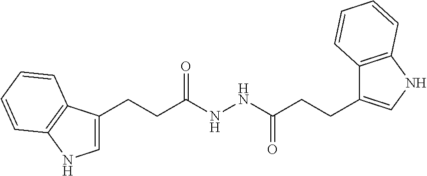 | 374.44 | 99 | 25.000 | 25.000 | 25.000 |
| 115 | 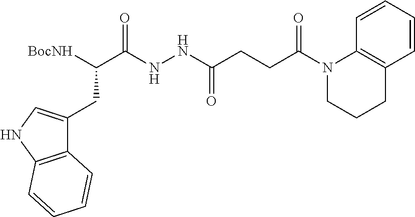 | 533.62 | 90 | 25.000 | 25.000 | 25.000 |
| 116 | 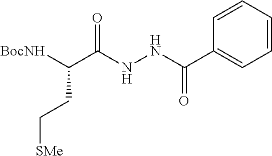 | 367.46 | >95 | 25.000 | 25.000 | 25.000 |
| 117 | 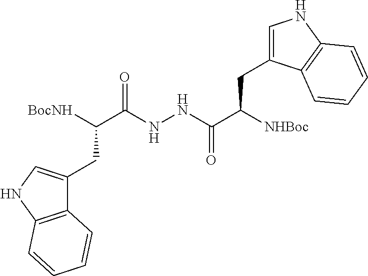 | 604.70 | >95 | 25.000 | 25.000 | 25.000 |

TABLE 4
Acyl hydrazines.

| | | | | Cat L | Cat B | Cat S |
|---|---|---|---|---|---|---|
| Compound No. | Structure | MW | Purity (%) | (μM) | (μM) | (μM) |
| 118 | 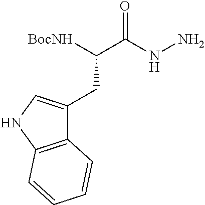 | 318.37 | >95 | 25.000 | 25.000 | 25.000 |
| 119 | 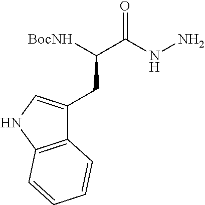 | 318.37 | >95 | 25.000 | 25.000 | 25.000 |

TABLE 5
N-Hydroxy-amides.

| Compound. | | | Purity | Cat L | Cat B | Cat S |
|---|---|---|---|---|---|---|
| No. | Structure | MW | (%) | (μM) | (μM) | (μM) |
| 120 | 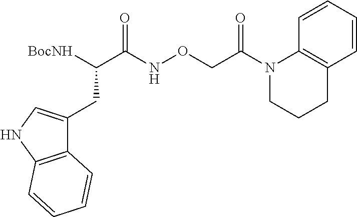 | 492.57 | >95 | 25.000 | 25.000 | 25.000 |

TABLE 6
Dialdehydes.

| Compound. | | | Purity | Cat L | Cat B | Cat S |
|---|---|---|---|---|---|---|
| No. | Structure | MW | (%) | (μM) | (μM) | (μM) |
| 121 | 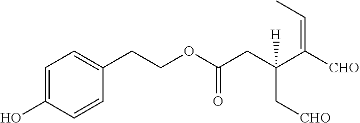 | 304.34 | 99 | 25.000 | 25.000 | 25.000 |

TABLE 7
Acyl hydrazines.

| Compound. | | | Purity | Cat L | Cat B | Cat S |
|---|---|---|---|---|---|---|
| No. | Structure | MW | (%) | (μM) | (μM) | (μM) |
| 122 | 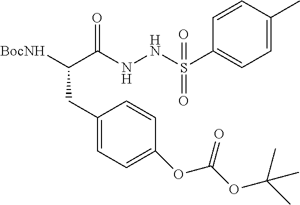 | 549.64 | >95 | 25.000 | 25.000 | 25.000 |

TABLE 8
Acyl hydrazones.

| Compound. | | | Purity | Cat L | Cat B | Cat S |
|---|---|---|---|---|---|---|
| No. | Structure | MW | (%) | (μM) | (μM) | (μM) |
| 123 | 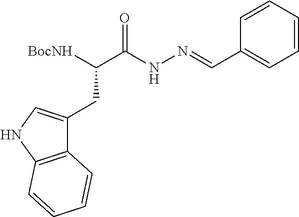 | 406.48 | >95 | 25.000 | 25.000 | 25.000 |
| 124 | 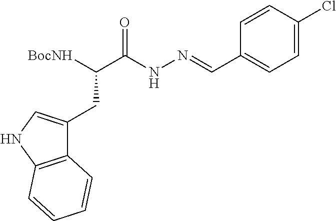 | 440.92 | >95 | 25.000 | 25.000 | 25.000 |

TABLE 9
Acyl hydrazine carboxamides.

| Compound. | | | Purity | Cat L | Cat B | Cat S |
|---|---|---|---|---|---|---|
| No. | Structure | MW | (%) | (μM) | (μM) | (μM) |
| 125 | 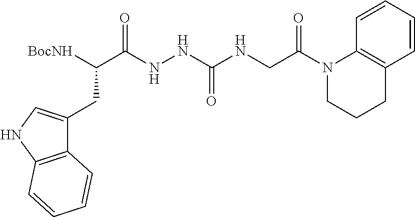 | 534.61 | >95 | 2.343 | 25.000 | 13.000 |

TABLE 10
Acyl hydrazine carbodithioates.

| Compound. | | | Purity | Cat L | Cat B | Cat S |
|---|---|---|---|---|---|---|
| No. | Structure | MW | (%) | (μM) | (μM) | (μM) |
| 126 | 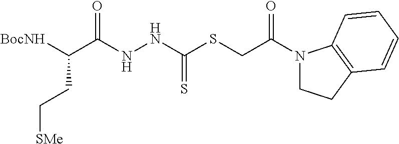 | 498.68 | >95 | 25.000 | 25.000 | 25.000 |

TABLE 11
Acyl hydrazine oxoacetamides.

| Compound. | | | Purity | Cat L | Cat B | Cat S |
|---|---|---|---|---|---|---|
| No. | Structure | MW | (%) | (μM) | (μM) | (μM) |
| 127 | 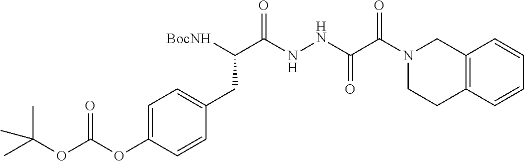 | 582.68 | >95 | 25.000 | 25.000 | 25.000 |

### B. Isomerism of Compounds of the Invention

Compounds included in the invention may comprise chiral centers which result in optical isomerism. The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. See March, Advanced Organic Chemistry, 4^{th} Ed., (1992), p. 109. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 1, the Cahn-Ingold-Prelog ranking is A>B>C>D. The lowest ranking atom, D is oriented away from the viewer.

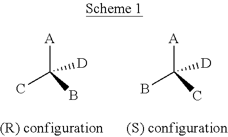

The present invention is meant to encompass the use of compounds comprising optical isomers, as well as their racemic and resolved, diastereomerically and enantiomerically pure forms and salts thereof. Diastereomers result from the presence of more than one chiral center in a compound. Diastereomeric pairs may be resolved by known separation techniques including normal and reverse phase chromatography, and crystallization.

By “isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound having the structure of Formula I, or a chiral intermediate thereof, is separated into 99% wt. % pure optical isomers by HPLC using a suitable chiral column, such as a member of the series of DAICEL CHIRALPAK® family of columns (Daicel Chemical Industries, Ltd., Tokyo, Japan). The column is operated according to the manufacturer's instructions.

### C. Preparation of Compounds of the Invention

Compounds useful in the practice of the invention may be prepared via synthetic organic chemistry methods well known to one of ordinary skill in the are. See March, 1992, Advanced Organic Chemistry, John Wiley & Sons. Inc., New York, N.Y., 4th ed.; Stewart et al., 1984, Solid Phase Peptide Synthesis, Pierce Chemical Company, Rockford, Ill., 2^{nd} ed.; and Harlow et al., 1998, Using Antibodies, A Laboratory Manual, Cold Spring Harbor Laboratory Press, Cold Spring Harbor, N.Y.

The strategy involved the design of a library containing a thiocarbazate scaffold incorporating a variety of functional groups at three different positions A, B and C, as shown in Scheme 2. From the outset, optimal diversity of the final products was sought in terms of size, shape and functionality. At the same time, optimal physical properties were maintained to ensure solubility, permeability and other “drug-like” properties. Finally, a strict requirement was adhered to for an expedient synthesis that would produce a minimum of 10 mg of final product in purities of at least 95% as determined by LC/MS analysis. Modifications at the A position involved changes in size, as well as replacement of the t-butyloxycarbonyl group. Position B underwent the most extensive modifications, where changes in size, polarity, acidity, and functionality were incorporated. Thiocarbazates derived from natural amino acids, such as methionine, valine, alanine, glutamic acid, leucine, proline, phenylalanine, tyrosine, threonine, serine, glutamic acid, lysine, arginine and histidine, along with unnatural amino acids were prepared. Modifications at C involved incorporation of ring constraints, removal of the amide bond and exploration of size requirements; a variety of acetamides derived from aniline, primary amines, and methyl esters were included. Examples of substituents at position C include differentially substituted anilines, quinolines and isoquinolines, non-aromatic amines, morpholines, indoline, and pyridinone.

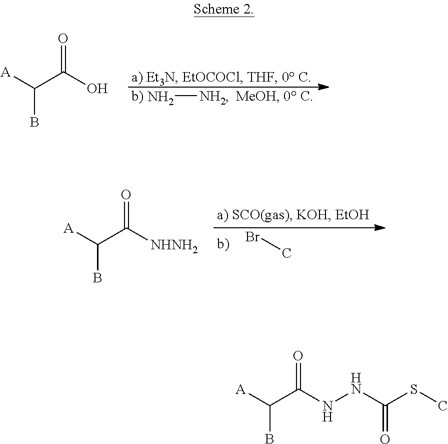

### D. Salts of Compounds of the Invention

Compounds of the present invention may take the form of salts. The term “salts,” embraces addition salts of free acids or free bases which are compounds of the invention. The term “pharmaceutically-acceptable salt” refers to salts which possess toxicity profiles within a range that affords utility in pharmaceutical applications.

Suitable pharmaceutically-acceptable acid addition salts may be prepared from an inorganic acid or from an organic acid. Examples of inorganic acids include hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, salicyclic, salicyclic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, toluenesulfonic, sulfanilic, cyclohexylaminosulfonic, stearic, algenic, γ-hydroxybutyric, salicyclic, galactaric and galacturonic acid.

Suitable pharmaceutically-acceptable base addition salts of compounds of the invention include for example, metallic salts including alkali metal, alkaline earth metal and transition metal salts such as, for example, calcium, magnesium, potassium, sodium and zinc salts. Pharmaceutically-acceptable base addition salts also include organic salts made from basic amines such as, for example, N,N′-dibenzylethylenediamine, chloroprocaine, choline, diethanolamine, ethylenediamine, meglumine (N-methylglucamine) and procaine. All of these salts may be prepared by conventional means from the corresponding compound according to Formulas I-105 as listed in Tables 1-11 by reacting, for example, the appropriate acid or base with the compound according to the Formula.

### E. Dosage and Administration

The invention encompasses the use of a pharmaceutical composition comprising at least one cathepsin L inhibitor, at least one cathepsin B inhibitor, at least one cathepsin S inhibitor, and any combination thereof, in a pharmaceutically-acceptable carrier to practice the methods of the invention.

As used herein, the term “pharmaceutically-acceptable carrier” means a chemical composition with which an inhibitor of a cathepsin may be combined and which, following the combination, can be used to administer an inhibitor of cathepsin L, cathepsin B, cathepsin S, or any combination thereof, to a mammal.

The pharmaceutical compositions useful for practicing the invention may be administered to deliver a dose of between 1 ng/kg/day and 100 mg/kg/day including all whole or partial integers there between. In one embodiment, the invention envisions administering a daily oral dose of 250 milligram to 1000 milligram of an inhibitor of cathepsin L, cathepsin, B, cathepsin, S or any combination thereof to an individual afflicted with a disease or disorder that would benefit from the inhibition of cathepsin L, cathepsin B, cathepsin S, or any combination thereof. In another embodiment, the invention envisions administering an inhalation dose of 1 milligram to 250 milligram daily of an inhibitor of cathepsin L, cathepsin B, cathepsin S, or any combination thereof to an individual in need thereof. In one aspect of the invention, the inhibitor is at least one thiocarbazate selected from the compounds listed in Table 1 or a salt thereof. In another aspect of the invention, the inhibitor is a oxacarbazate selected from the list of compounds listed in Table 2, or a salt thereof. In another aspect of the invention, the inhibitor is a diacyl hydrazine selected from the list of compounds listed in Table 3. In another aspect of the invention, the inhibitor is an acyl hydrazine selected from the list of compounds listed in Table 4. In another aspect of the invention, the inhibitor is an N-hydroxy-amide selected from the list of compounds listed in Table 5. In another aspect of the invention, the inhibitor is a dialdehyde selected from the list of compounds listed in Table 6. In another aspect of the invention, the inhibitor is a sulfonylated acyl hydrazine selected from the list of compounds listed in Table 7. In another aspect of the invention, the inhibitor is a acyl hydrazone selected from the list of compounds listed in Table 8. In another aspect of the invention, the inhibitor is an acyl hydrazine carboxamide selected from the list of compounds listed in Table 9. In another aspect of the invention, the inhibitor is an acyl hydrazine carbodithioate selected from the list of compounds listed in Table 10. In another aspect of the invention, the inhibitor is an acyl hydrazine oxoacetamide selected from the list of compounds listed in Table 11.

Pharmaceutical compositions that are useful in the methods of the invention may be administered systemically in oral solid formulations, ophthalmic, suppository, aerosol, topical or other similar formulations. In addition to an inhibitor of cathepsin L, cathepsin B, cathepsin S or any combination thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an inhibitor of cathepsin L according to the methods of the invention.

Compounds which are identified using any of the methods described herein may be formulated and administered to a mammal for treatment of the diseases disclosed herein are now described.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates. Further, the present invention contemplates administering a pharmacological composition of the present invention to a zoonotic life cycle reservoir that acts as a vector for transmission of a virus including SARS, Ebola, or Hendra, to humans.

Pharmaceutical compositions that are useful in the methods of the invention may be prepared, packaged, or sold in formulations suitable for oral, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

### II. Methods

### Methods of Use of Cathepsin L Inhibitors

In one embodiment, the invention includes a method of treating a subject afflicted with or at risk of a disease or disorder affecting bone and cartilage remodeling, the method comprising administering a therapeutically effective amount of at least one cathepsin L inhibitor to the subject in need thereof, where the cathepsin L inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin L inhibitor of the invention comprises a thiocarbazate. In another embodiment, a thiocarbazate cathepsin L inhibitor of the invention comprises a compound of Compound No. 11, 5-92 (Table 1). In another embodiment, a cathepsin L inhibitor of the invention is a thiocarbazate selected from the group of compounds consisting of Compound No. 1, 5, 7, 8-15, 19-24, 29, 31, 34, 36-43, 46, 47, 50-56, 58, 62, 76-78, and 83 (Table 1). In another embodiment, a cathepsin L inhibitor of the invention is an oxacarbazate selected from the group of compounds consisting of Compound No. 93-112 (Table 2). In another embodiment a cathepsin L inhibitor of the invention is an oxacarbazate selected from the group of compounds consisting of Compound No. 93, 94, and 96 (Table 2). In yet another embodiment, a cathepsin L inhibitor of the invention is a diacyl hydrazine selected from the group of compounds consisting of Compound No. 113-117 (Table 3). In yet another embodiment, a cathepsin L inhibitor of the invention is an acyl hydrazine selected from the group of compounds consisting of Compound No. 118-119 (Table 4). In yet another embodiment, a cathepsin L inhibitor of the invention is an N-hydroxy-amide consisting of Compound No. 120 (Table 5). In yet another embodiment, a cathepsin L inhibitor of the invention is a dialdehyde consisting of Compound No. 121 (Table 6). In yet another embodiment, a cathepsin L inhibitor of the invention is a sulfonylated acyl hydrazine consisting of Compound No. 122 (Table 7). In yet another embodiment, a cathepsin L inhibitor of the invention is a acyl hydrazone selected from the group of compounds consisting of Compound No. 123-124 (Table 8). In yet another embodiment, a cathepsin L inhibitor of the invention is a acyl hydrazine carboxamide consisting of Compound No. 125 (Table 9). In yet another embodiment, a cathepsin L inhibitor of the invention is a acyl hydrazine carbodithioate consisting of Compound No. 126 (Table 10). In yet another embodiment, a cathepsin L inhibitor of the invention is an acyl hydrazine oxoacetamide consisting of Compound No. 127 (Table 11).

In another embodiment, the invention includes a method of inhibiting viral entry into mammalian cells, the method comprising administering a therapeutically effective amount of at least one cathepsin L inhibitor to the subject in need thereof, where the cathepsin L inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin L inhibitor of the invention comprises a thiocarbazate. In another embodiment, a thiocarbazate cathepsin L inhibitor of the invention comprises a compound of Compound No. 11, 5-92 (Table 1). In another embodiment, a cathepsin L inhibitor of the invention is a thiocarbazate selected from the group of compounds consisting of Compound No. 1, 5, 7, 8-15, 19-24, 29, 31, 34, 36-43, 46, 47, 50-56, 58, 62, 76-78, and 83 (Table 1). In another embodiment, a cathepsin L inhibitor of the invention is an oxacarbazate selected from the group of compounds consisting of Compound No. 93-112 (Table 2). In another embodiment a cathepsin L inhibitor of the invention is an oxacarbazate selected from the group of compounds consisting of Compound No. 93, 94, and 96 (Table 2). In yet another embodiment, a cathepsin L inhibitor of the invention is a diacyl hydrazine selected from the group of compounds consisting of Compound No. 113-117 (Table 3). In yet another embodiment, a cathepsin L inhibitor of the invention is an acyl hydrazine selected from the group of compounds consisting of Compound No. 118-119 (Table 4). In yet another embodiment, a cathepsin L inhibitor of the invention is an N-hydroxy-amide consisting of Compound No. 120 (Table 5). In yet another embodiment, a cathepsin L inhibitor of the invention is a dialdehyde consisting of Compound No. 121 (Table 6). In yet another embodiment, a cathepsin L inhibitor of the invention is a sulfonylated acyl hydrazine consisting of Compound No. 122 (Table 7). In yet another embodiment, a cathepsin L inhibitor of the invention is a acyl hydrazone selected from the group of compounds consisting of Compound No. 123-124 (Table 8). In yet another embodiment, a cathepsin L inhibitor of the invention is a acyl hydrazine carboxamide consisting of Compound No. 125 (Table 9). In yet another embodiment, a cathepsin L inhibitor of the invention is a acyl hydrazine carbodithioate consisting of Compound No. 126 (Table 10). In yet another embodiment, a cathepsin L inhibitor of the invention is an acyl hydrazine oxoacetamide consisting of Compound No. 127 (Table 11).

In another embodiment, the invention provides a method of treating a subject infected by a viral pathogen, the method comprising administering a therapeutically effective amount of at least one cathepsin L inhibitor to the subject in need thereof, where the cathepsin L inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin L inhibitor of the invention comprises a thiocarbazate. In another embodiment, a thiocarbazate cathepsin L inhibitor of the invention comprises a compound of Compound No. 11, 5-92 (Table 1). In another embodiment, a cathepsin L inhibitor of the invention is a thiocarbazate selected from the group of compounds consisting of Compound No. 1, 5, 7, 8-15, 19-24, 29, 31, 34, 36-43, 46, 47, 50-56, 58, 62, 76-78, and 83 (Table 1). In another embodiment, a cathepsin L inhibitor of the invention is an oxacarbazate selected from the group of compounds consisting of Compound No. 93-112 (Table 2). In another embodiment a cathepsin L inhibitor of the invention is an oxacarbazate selected from the group of compounds consisting of Compound No. 93, 94, and 96 (Table 2). In yet another embodiment, a cathepsin L inhibitor of the invention is a diacyl hydrazine selected from the group of compounds consisting of Compound No. 113-117 (Table 3). In yet another embodiment, a cathepsin L inhibitor of the invention is an acyl hydrazine selected from the group of compounds consisting of Compound No. 118-119 (Table 4). In yet another embodiment, a cathepsin L inhibitor of the invention is an N-hydroxy-amide consisting of Compound No. 120 (Table 5). In yet another embodiment, a cathepsin L inhibitor of the invention is a dialdehyde consisting of Compound No. 121 (Table 6). In yet another embodiment, a cathepsin L inhibitor of the invention is a sulfonylated acyl hydrazine consisting of Compound No. 122 (Table 7). In yet another embodiment, a cathepsin L inhibitor of the invention is a acyl hydrazone selected from the group of compounds consisting of Compound No. 123-124 (Table 8). In yet another embodiment, a cathepsin L inhibitor of the invention is a acyl hydrazine carboxamide consisting of Compound No. 125 (Table 9). In yet another embodiment, a cathepsin L inhibitor of the invention is a acyl hydrazine carbodithioate consisting of Compound No. 126 (Table 10). In yet another embodiment, a cathepsin L inhibitor of the invention is an acyl hydrazine oxoacetamide consisting of Compound No. 127 (Table 11).

In yet another embodiment, the invention provides a method of treating a subject at risk of developing a viral infection where the method includes prophylactically administering a therapeutically effective amount of at least one cathepsin L inhibitor to the subject in need thereof, where the cathepsin L inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin L inhibitor of the invention comprises a thiocarbazate. In another embodiment, a thiocarbazate cathepsin L inhibitor of the invention comprises a compound of Compound No. 11, 5-92 (Table 1). In another embodiment, a cathepsin L inhibitor of the invention is a thiocarbazate selected from the group of compounds consisting of Compound No. 1, 5, 7, 8-15, 19-24, 29, 31, 34, 36-43, 46, 47, 50-56, 58, 62, 76-78, and 83 (Table 1). In another embodiment, a cathepsin L inhibitor of the invention is an oxacarbazate selected from the group of compounds consisting of Compound No. 93-112 (Table 2). In another embodiment a cathepsin L inhibitor of the invention is an oxacarbazate selected from the group of compounds consisting of Compound No. 93, 94, and 96 (Table 2). In yet another embodiment, a cathepsin L inhibitor of the invention is a diacyl hydrazine selected from the group of compounds consisting of Compound No. 113-117 (Table 3). In yet another embodiment, a cathepsin L inhibitor of the invention is an acyl hydrazine selected from the group of compounds consisting of Compound No. 118-119 (Table 4). In yet another embodiment, a cathepsin L inhibitor of the invention is an N-hydroxy-amide consisting of Compound No. 120 (Table 5). In yet another embodiment, a cathepsin L inhibitor of the invention is a dialdehyde consisting of Compound No. 121 (Table 6). In yet another embodiment, a cathepsin L inhibitor of the invention is a sulfonylated acyl hydrazine consisting of Compound No. 122 (Table 7). In yet another embodiment, a cathepsin L inhibitor of the invention is a acyl hydrazone selected from the group of compounds consisting of Compound No. 123-124 (Table 8). In yet another embodiment, a cathepsin L inhibitor of the invention is a acyl hydrazine carboxamide consisting of Compound No. 125 (Table 9). In yet another embodiment, a cathepsin L inhibitor of the invention is a acyl hydrazine carbodithioate consisting of Compound No. 126 (Table 10). In yet another embodiment, a cathepsin L inhibitor of the invention is an acyl hydrazine oxoacetamide consisting of Compound No. 127 (Table 11). For either treatment or prevention, the infection is preferably viral, and more preferably SARS, Ebola, or Hendra virus.

In another embodiment, the invention provides a method of treating a subject infected by a non-viral pathogen where the method includes administering a therapeutically effective amount of at least one cathepsin L inhibitor to the subject in need thereof, where the cathepsin L inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin L inhibitor of the invention comprises a thiocarbazate. In another embodiment, a thiocarbazate cathepsin L inhibitor of the invention comprises a compound of Compound No. 11, 5-92 (Table 1). In another embodiment, a cathepsin L inhibitor of the invention is a thiocarbazate selected from the group of compounds consisting of Compound No. 1, 5, 7, 8-15, 19-24, 29, 31, 34, 36-43, 46, 47, 50-56, 58, 62, 76-78, and 83 (Table 1). In another embodiment, a cathepsin L inhibitor of the invention is an oxacarbazate selected from the group of compounds consisting of Compound No. 93-112 (Table 2). In another embodiment a cathepsin L inhibitor of the invention is an oxacarbazate selected from the group of compounds consisting of Compound No. 93, 94, and 96 (Table 2). In yet another embodiment, a cathepsin L inhibitor of the invention is a diacyl hydrazine selected from the group of compounds consisting of Compound No. 113-117 (Table 3). In yet another embodiment, a cathepsin L inhibitor of the invention is an acyl hydrazine selected from the group of compounds consisting of Compound No. 118-119 (Table 4). In yet another embodiment, a cathepsin L inhibitor of the invention is an N-hydroxy-amide consisting of Compound No. 120 (Table 5). In yet another embodiment, a cathepsin L inhibitor of the invention is a dialdehyde consisting of Compound No. 121 (Table 6). In yet another embodiment, a cathepsin L inhibitor of the invention is a sulfonylated acyl hydrazine consisting of Compound No. 122 (Table 7). In yet another embodiment, a cathepsin L inhibitor of the invention is an acyl hydrazone selected from the group of compounds consisting of Compound No. 123-124 (Table 8). In yet another embodiment, a cathepsin L inhibitor of the invention is a acyl hydrazine carboxamide consisting of Compound No. 125 (Table 9). In yet another embodiment, a cathepsin L inhibitor of the invention is a acyl hydrazine carbodithioate consisting of Compound No. 126 (Table 10). In yet another embodiment, a cathepsin L inhibitor of the invention is an acyl hydrazine oxoacetamide consisting of Compound No. 127 (Table 11).

In yet another embodiment, the invention provides a method of treating a subject at risk of developing a non-viral pathogen infection where the method includes prophylactically administering a therapeutically effective amount of at least one cathepsin L inhibitor to the subject in need thereof, where the cathepsin L inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin L inhibitor of the invention comprises a thiocarbazate. In another embodiment, a thiocarbazate cathepsin L inhibitor of the invention comprises a compound of Compound No. 11, 5-92 (Table 1). In another embodiment, a cathepsin L inhibitor of the invention is a thiocarbazate selected from the group of compounds consisting of Compound No. 1, 5, 7, 8-15, 19-24, 29, 31, 34, 36-43, 46, 47, 50-56, 58, 62, 76-78, and 83 (Table 1). In another embodiment, a cathepsin L inhibitor of the invention is an oxacarbazate selected from the group of compounds consisting of Compound No. 93-112 (Table 2). In another embodiment a cathepsin L inhibitor of the invention is an oxacarbazate selected from the group of compounds consisting of Compound No. 93, 94, and 96 (Table 2). In yet another embodiment, a cathepsin L inhibitor of the invention is a diacyl hydrazine selected from the group of compounds consisting of Compound No. 113-117 (Table 3). In yet another embodiment, a cathepsin L inhibitor of the invention is an acyl hydrazine selected from the group of compounds consisting of Compound No. 118-119 (Table 4). In yet another embodiment, a cathepsin L inhibitor of the invention is an N-hydroxy-amide consisting of Compound No. 120 (Table 5). In yet another embodiment, a cathepsin L inhibitor of the invention is a dialdehyde consisting of Compound No. 121 (Table 6). In yet another embodiment, a cathepsin L inhibitor of the invention is a sulfonylated acyl hydrazine consisting of Compound No. 122 (Table 7). In yet another embodiment, a cathepsin L inhibitor of the invention is an acyl hydrazone selected from the group of compounds consisting of Compound No. 123-124 (Table 8). In yet another embodiment, a cathepsin L inhibitor of the invention is an acyl hydrazine carboxamide consisting of Compound No. 125 (Table 9). In yet another embodiment, a cathepsin L inhibitor of the invention is a acyl hydrazine carbodithioate consisting of Compound No. 126 (Table 10). In yet another embodiment, a cathepsin L inhibitor of the invention is an acyl hydrazine oxoacetamide consisting of Compound No. 127 (Table 11). For either treatment or prevention, the infection is preferably parasitic.

In still another embodiment, the invention provides a method of treating a subject afflicted with or at risk of hair loss where the method includes prophylactically administering a therapeutically effective amount of at least one cathepsin L inhibitor to the subject in need thereof, where the cathepsin L inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin L inhibitor of the invention comprises a thiocarbazate. In another embodiment, a thiocarbazate cathepsin L inhibitor of the invention comprises a compound of Compound No. 11, 5-92 (Table 1). In another embodiment, a cathepsin L inhibitor of the invention is a thiocarbazate selected from the group of compounds consisting of Compound No. 1, 5, 7, 8-15, 19-24, 29, 31, 34, 36-43, 46, 47, 50-56, 58, 62, 76-78, and 83 (Table 1). In another embodiment, a cathepsin L inhibitor of the invention is an oxacarbazate selected from the group of compounds consisting of Compound No. 93-112 (Table 2). In another embodiment a cathepsin L inhibitor of the invention is an oxacarbazate selected from the group of compounds consisting of Compound No. 93, 94, and 96 (Table 2). In yet another embodiment, a cathepsin L inhibitor of the invention is a diacyl hydrazine selected from the group of compounds consisting of Compound No. 113-117 (Table 3). In yet another embodiment, a cathepsin L inhibitor of the invention is an acyl hydrazine selected from the group of compounds consisting of Compound No. 118-119 (Table 4). In yet another embodiment, a cathepsin L inhibitor of the invention is an N-hydroxy-amide consisting of Compound No. 120 (Table 5). In yet another embodiment, a cathepsin L inhibitor of the invention is a dialdehyde consisting of Compound No. 121 (Table 6). In yet another embodiment, a cathepsin L inhibitor of the invention is a sulfonylated acyl hydrazine consisting of Compound No. 122 (Table 7). In yet another embodiment, a cathepsin L inhibitor of the invention is a acyl hydrazone selected from the group of compounds consisting of Compound No. 123-124 (Table 8). In yet another embodiment, a cathepsin L inhibitor of the invention is a acyl hydrazine carboxamide consisting of Compound No. 125 (Table 9). In yet another embodiment, a cathepsin L inhibitor of the invention is a acyl hydrazine carbodithioate consisting of Compound No. 126 (Table 10). In yet another embodiment, a cathepsin L inhibitor of the invention is an acyl hydrazine oxoacetamide consisting of Compound No. 127 (Table 11).

Although the method of the invention requires the administration of at least one cathepsin L inhibitor, two, three, four, five, six, seven, eight, nine, ten, or more cathepsin L inhibitors may also be used. Further, the skilled artisan will appreciate that the methods of the invention include administering to a subject in need thereof a therapeutically effective amount of at least one cathepsin L inhibitor in combination with at least one cathepsin B inhibitor, at least one cathepsin S inhibitor, or any combination thereof.

### Methods of Use of Cathepsin B Inhibitors

In one embodiment, the invention includes a method of treating a subject afflicted with or at risk of developing cancer, the method comprising administering a therapeutically effective amount of at least one cathepsin B inhibitor to a subject in need thereof, where the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin B inhibitor of the invention is a thiocarbazate selected from the group of compounds consisting of Compound No. 1 and 5-92 (Table 1). In another embodiment, a cathepsin B inhibitor of the invention is a thiocarbazate selected from the group of compounds consisting of Compound No. 21-23, 29, 31, 41, 44, 45, 57, 65, 66, 78 and 79 (Table 1). In another embodiment, a cathepsin B inhibitor of the invention is an oxacarbazate selected from the group of compounds consisting of Compound No. 93-112 (Table 2). In yet another embodiment, a cathepsin B inhibitor of the invention is a diacyl hydrazine selected from the group of compounds consisting of Compound No. 113-117 (Table 3). In yet another embodiment, a cathepsin B inhibitor of the invention is an acyl hydrazine selected from the group of compounds consisting of Compound No. 118-119 (Table 4). In yet another embodiment, a cathepsin B inhibitor of the invention is an N-hydroxy-amide consisting of Compound No. 120 (Table 5). In yet another embodiment, a cathepsin B inhibitor of the invention is a dialdehyde consisting of Compound No. 121 (Table 6). In yet another embodiment, a cathepsin B inhibitor of the invention is a sulfonylated acyl hydrazine consisting of Compound No. 122 (Table 7). In yet another embodiment, a cathepsin B inhibitor of the invention is a acyl hydrazone selected from the group of compounds consisting of Compound No. 123-124 (Table 8). In yet another embodiment, a cathepsin B inhibitor of the invention is a acyl hydrazine carboxamide consisting of Compound No. 125 (Table 9). In yet another embodiment, a cathepsin B inhibitor of the invention is an acyl hydrazine carbodithioate consisting of Compound No. 126 (Table 10). In yet another embodiment, a cathepsin B inhibitor of the invention is an acyl hydrazine oxoacetamide consisting of Compound No. 127 (Table 11).

In another embodiment, the invention includes a method of treating a subject afflicted with or at risk of developing osteoporosis, the method comprising administering a therapeutically effective amount of at least one cathepsin B inhibitor to a subject in need thereof, where the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin B inhibitor of the invention is a thiocarbazate selected from the group of compounds consisting of Compound No. 1 and 5-92 (Table 1). In another embodiment, a cathepsin B inhibitor of the invention is a thiocarbazate selected from the group of compounds consisting of Compound No. 21-23, 29, 31, 41, 44, 45, 57, 65, 66, 78 and 79 (Table 1). In another embodiment, a cathepsin B inhibitor of the invention is an oxacarbazate selected from the group of compounds consisting of Compound No. 93-112 (Table 2). In yet another embodiment, a cathepsin B inhibitor of the invention is a diacyl hydrazine selected from the group of compounds consisting of Compound No. 113-117 (Table 3). In yet another embodiment, a cathepsin B inhibitor of the invention is an acyl hydrazine selected from the group of compounds consisting of Compound No. 118-119 (Table 4). In yet another embodiment, a cathepsin B inhibitor of the invention is an N-hydroxy-amide consisting of Compound No. 120 (Table 5). In yet another embodiment, a cathepsin B inhibitor of the invention is a dialdehyde consisting of Compound No. 121 (Table 6). In yet another embodiment, a cathepsin B inhibitor of the invention is a sulfonylated acyl hydrazine consisting of Compound No. 122 (Table 7). In yet another embodiment, a cathepsin B inhibitor of the invention is a acyl hydrazone selected from the group of compounds consisting of Compound No. 123-124 (Table 8). In yet another embodiment, a cathepsin B inhibitor of the invention is a acyl hydrazine carboxamide consisting of Compound No. 125 (Table 9). In yet another embodiment, a cathepsin B inhibitor of the invention is a acyl hydrazine carbodithioate consisting of Compound No. 126 (Table 10). In yet another embodiment, a cathepsin B inhibitor of the invention is an acyl hydrazine oxoacetamide consisting of Compound No. 127 (Table 11).

In another embodiment, the invention includes a method of treating a subject afflicted with or at risk of developing arthritis, the method comprising administering a therapeutically effective amount of at least one cathepsin B inhibitor to a subject in need thereof, where the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin B inhibitor of the invention is a thiocarbazate selected from the group of compounds consisting of Compound No. 1 and 5-92 (Table 1). In another embodiment, a cathepsin B inhibitor of the invention is a thiocarbazate selected from the group of compounds consisting of Compound No. 21-23, 29, 31, 41, 44, 45, 57, 65, 66, 78 and 79 (Table 1). In another embodiment, a cathepsin B inhibitor of the invention is an oxacarbazate selected from the group of compounds consisting of Compound No. 93-112 (Table 2). In yet another embodiment, a cathepsin B inhibitor of the invention is a diacyl hydrazine selected from the group of compounds consisting of Compound No. 113-117 (Table 3). In yet another embodiment, a cathepsin B inhibitor of the invention is an acyl hydrazine selected from the group of compounds consisting of Compound No. 118-119 (Table 4). In yet another embodiment, a cathepsin B inhibitor of the invention is an N-hydroxy-amide consisting of Compound No. 120 (Table 5). In yet another embodiment, a cathepsin B inhibitor of the invention is a dialdehyde consisting of Compound No. 121 (Table 6). In yet another embodiment, a cathepsin B inhibitor of the invention is a sulfonylated acyl hydrazine consisting of Compound No. 122 (Table 7). In yet another embodiment, a cathepsin B inhibitor of the invention is a acyl hydrazone selected from the group of compounds consisting of Compound No. 123-124 (Table 8). In yet another embodiment, a cathepsin B inhibitor of the invention is a acyl hydrazine carboxamide consisting of Compound No. 125 (Table 9). In yet another embodiment, a cathepsin B inhibitor of the invention is a acyl hydrazine carbodithioate consisting of Compound No. 126 (Table 10). In yet another embodiment, a cathepsin B inhibitor of the invention is an acyl hydrazine oxoacetamide consisting of Compound No. 127 (Table 11).

In another embodiment, the invention includes a method of inhibiting viral entry into mammalian cells, the method comprising administering a therapeutically effective amount of at least one cathepsin B inhibitor to the subject in need thereof, where the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin B inhibitor of the invention is a thiocarbazate selected from the group of compounds consisting of Compound No. 1 and 5-92 (Table 1). In another embodiment, a cathepsin B inhibitor of the invention is a thiocarbazate selected from the group of compounds consisting of Compound No. 21-23, 29, 31, 41, 44, 45, 57, 65, 66, 78 and 79 (Table 1). In another embodiment, a cathepsin B inhibitor of the invention is an oxacarbazate selected from the group of compounds consisting of Compound No. 93-112 (Table 2). In yet another embodiment, a cathepsin B inhibitor of the invention is a diacyl hydrazine selected from the group of compounds consisting of Compound No. 113-117 (Table 3). In yet another embodiment, a cathepsin B inhibitor of the invention is an acyl hydrazine selected from the group of compounds consisting of Compound No. 118-119 (Table 4). In yet another embodiment, a cathepsin B inhibitor of the invention is an N-hydroxy-amide consisting of Compound No. 120 (Table 5). In yet another embodiment, a cathepsin B inhibitor of the invention is a dialdehyde consisting of Compound No. 121 (Table 6). In yet another embodiment, a cathepsin B inhibitor of the invention is a sulfonylated acyl hydrazine consisting of Compound No. 122 (Table 7). In yet another embodiment, a cathepsin B inhibitor of the invention is a acyl hydrazone selected from the group of compounds consisting of Compound No. 123-124 (Table 8). In yet another embodiment, a cathepsin B inhibitor of the invention is a acyl hydrazine carboxamide consisting of Compound No. 125 (Table 9). In yet another embodiment, a cathepsin B inhibitor of the invention is a acyl hydrazine carbodithioate consisting of Compound No. 126 (Table 10). In yet another embodiment, a cathepsin B inhibitor of the invention is an acyl hydrazine oxoacetamide consisting of Compound No. 127 (Table 11).

In another embodiment, the invention provides a method of treating a subject infected by a viral pathogen, the method comprising administering a therapeutically effective amount of at least one cathepsin B inhibitor to the subject in need thereof, where the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin B inhibitor of the invention is a thiocarbazate selected from the group of compounds consisting of Compound No. 1 and 5-92 (Table 1). In another embodiment, a cathepsin B inhibitor of the invention is a thiocarbazate selected from the group of compounds consisting of Compound No. 21-23, 29, 31, 41, 44, 45, 57, 65, 66, 78 and 79 (Table 1). In another embodiment, a cathepsin B inhibitor of the invention is an oxacarbazate selected from the group of compounds consisting of Compound No. 93-112 (Table 2). In yet another embodiment, a cathepsin B inhibitor of the invention is a diacyl hydrazine selected from the group of compounds consisting of Compound No. 113-117 (Table 3). In yet another embodiment, a cathepsin B inhibitor of the invention is an acyl hydrazine selected from the group of compounds consisting of Compound No. 118-119 (Table 4). In yet another embodiment, a cathepsin B inhibitor of the invention is an N-hydroxy-amide consisting of Compound No. 120 (Table 5). In yet another embodiment, a cathepsin B inhibitor of the invention is a dialdehyde consisting of Compound No. 121 (Table 6). In yet another embodiment, a cathepsin B inhibitor of the invention is a sulfonylated acyl hydrazine consisting of Compound No. 122 (Table 7). In yet another embodiment, a cathepsin B inhibitor of the invention is a acyl hydrazone selected from the group of compounds consisting of Compound No. 123-124 (Table 8). In yet another embodiment, a cathepsin B inhibitor of the invention is a acyl hydrazine carboxamide consisting of Compound No. 125 (Table 9). In yet another embodiment, a cathepsin B inhibitor of the invention is a acyl hydrazine carbodithioate consisting of Compound No. 126 (Table 10). In yet another embodiment, a cathepsin B inhibitor of the invention is an acyl hydrazine oxoacetamide consisting of Compound No. 127 (Table 11).

In yet another embodiment, the invention provides a method of treating a subject at risk of developing a viral infection where the method includes prophylactically administering a therapeutically effective amount of at least one cathepsin B inhibitor to the subject in need thereof, where the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin B inhibitor of the invention is a thiocarbazate selected from the group of compounds consisting of Compound No. 1 and 5-92 (Table 1). In another embodiment, a cathepsin B inhibitor of the invention is a thiocarbazate selected from the group of compounds consisting of Compound No. 21-23, 29, 31, 41, 44, 45, 57, 65, 66, 78 and 79 (Table 1). In another embodiment, a cathepsin B inhibitor of the invention is an oxacarbazate selected from the group of compounds consisting of Compound No. 93-112 (Table 2). In yet another embodiment, a cathepsin B inhibitor of the invention is a diacyl hydrazine selected from the group of compounds consisting of Compound No. 113-117 (Table 3). In yet another embodiment, a cathepsin B inhibitor of the invention is an acyl hydrazine selected from the group of compounds consisting of Compound No. 118-119 (Table 4). In yet another embodiment, a cathepsin B inhibitor of the invention is an N-hydroxy-amide consisting of Compound No. 120 (Table 5). In yet another embodiment, a cathepsin B inhibitor of the invention is a dialdehyde consisting of Compound No. 121 (Table 6). In yet another embodiment, a cathepsin B inhibitor of the invention is a sulfonylated acyl hydrazine consisting of Compound No. 122 (Table 7). In yet another embodiment, a cathepsin B inhibitor of the invention is a acyl hydrazone selected from the group of compounds consisting of Compound No. 123-124 (Table 8). In yet another embodiment, a cathepsin B inhibitor of the invention is a acyl hydrazine carboxamide consisting of Compound No. 125 (Table 9). In yet another embodiment, a cathepsin B inhibitor of the invention is a acyl hydrazine carbodithioate consisting of Compound No. 126 (Table 10). In yet another embodiment, a cathepsin B inhibitor of the invention is an acyl hydrazine oxoacetamide consisting of Compound No. 127 (Table 11). For either treatment or prevention, the infection is preferably viral, and more preferably SARS, Ebola, or Hendra virus.

In another embodiment, the invention provides a method of treating a subject infected by a non-viral pathogen where the method includes administering a therapeutically effective amount of at least one cathepsin B inhibitor to the subject in need thereof, where the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin B inhibitor of the invention is a thiocarbazate selected from the group of compounds consisting of Compound No. 1 and 5-92 (Table 1). In another embodiment, a cathepsin B inhibitor of the invention is a thiocarbazate selected from the group of compounds consisting of Compound No. 21-23, 29, 31, 41, 44, 45, 57, 65, 66, 78 and 79 (Table 1). In another embodiment, a cathepsin B inhibitor of the invention is an oxacarbazate selected from the group of compounds consisting of Compound No. 93-112 (Table 2). In yet another embodiment, a cathepsin B inhibitor of the invention is a diacyl hydrazine selected from the group of compounds consisting of Compound No. 113-117 (Table 3). In yet another embodiment, a cathepsin B inhibitor of the invention is an acyl hydrazine selected from the group of compounds consisting of Compound No. 118-119 (Table 4). In yet another embodiment, a cathepsin B inhibitor of the invention is an N-hydroxy-amide consisting of Compound No. 120 (Table 5). In yet another embodiment, a cathepsin B inhibitor of the invention is a dialdehyde consisting of Compound No. 121 (Table 6). In yet another embodiment, a cathepsin B inhibitor of the invention is a sulfonylated acyl hydrazine consisting of Compound No. 122 (Table 7). In yet another embodiment, a cathepsin B inhibitor of the invention is a acyl hydrazone selected from the group of compounds consisting of Compound No. 123-124 (Table 8). In yet another embodiment, a cathepsin B inhibitor of the invention is a acyl hydrazine carboxamide consisting of Compound No. 125 (Table 9). In yet another embodiment, a cathepsin B inhibitor of the invention is a acyl hydrazine carbodithioate consisting of Compound No. 126 (Table 10). In yet another embodiment, a cathepsin B inhibitor of the invention is an acyl hydrazine oxoacetamide consisting of Compound No. 127 (Table 11). For either treatment or prevention, the infection is preferably parasitic.

In yet another embodiment, the invention provides a method of treating a subject at risk of developing a non-viral pathogen infection where the method includes prophylactically administering a therapeutically effective amount of at least one cathepsin B inhibitor to the subject in need thereof, where the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin B inhibitor of the invention is a thiocarbazate selected from the group of compounds consisting of Compound No. 1 and 5-92 (Table 1). In another embodiment, a cathepsin B inhibitor of the invention is a thiocarbazate selected from the group of compounds consisting of Compound No. 21-23, 29, 31, 41, 44, 45, 57, 65, 66, 78 and 79 (Table 1). In another embodiment, a cathepsin B inhibitor of the invention is an oxacarbazate selected from the group of compounds consisting of Compound No. 93-112 (Table 2). In yet another embodiment, a cathepsin B inhibitor of the invention is a diacyl hydrazine selected from the group of compounds consisting of Compound No. 113-117 (Table 3). In yet another embodiment, a cathepsin B inhibitor of the invention is an acyl hydrazine selected from the group of compounds consisting of Compound No. 118-119 (Table 4). In yet another embodiment, a cathepsin B inhibitor of the invention is an N-hydroxy-amide consisting of Compound No. 120 (Table 5). In yet another embodiment, a cathepsin B inhibitor of the invention is a dialdehyde consisting of Compound No. 121 (Table 6). In yet another embodiment, a cathepsin B inhibitor of the invention is a sulfonylated acyl hydrazine consisting of Compound No. 122 (Table 7). In yet another embodiment, a cathepsin B inhibitor of the invention is a acyl hydrazone selected from the group of compounds consisting of Compound No. 123-124 (Table 8). In yet another embodiment, a cathepsin B inhibitor of the invention is a acyl hydrazine carboxamide consisting of Compound No. 125 (Table 9). In yet another embodiment, a cathepsin B inhibitor of the invention is a acyl hydrazine carbodithioate consisting of Compound No. 126 (Table 10). In yet another embodiment, a cathepsin B inhibitor of the invention is an acyl hydrazine oxoacetamide consisting of Compound No. 127 (Table 11). For either treatment or prevention, the infection is preferably parasitic.

Although the methods of the invention require the administration of at least one cathepsin B inhibitor, two, three, four, five, six, seven, eight, nine, ten, or more cathepsin B inhibitors may also be used. Further, the skilled artisan will appreciate that the methods of the invention include administering to a subject in need thereof at least one cathepsin B inhibitor in combination with at least one cathepsin L inhibitor, at least one cathepsin S inhibitor, or an combination thereof.

The method of the invention may be practiced in any subject diagnosed with, or at risk of developing cancer, osteoporosis, arthritis, and/or an infection caused by a pathogen that relies on Cathepsin B expression or activity to maintain its infectivity and pathogenicity. Preferably, the subject is a mammal and more preferably, a human.

### Methods of Use of Cathepsin S Inhibitors

In one another embodiment, the invention includes a method of treating a subject afflicted with an autoimmune disease, the method comprising administering a therapeutically effective amount of at least one cathepsin S inhibitor to a subject in need thereof, where the cathepsin S inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin S inhibitor of the invention is a thiocarbazate selected from the group of compounds consisting of Compound No. 1 and 5-92 (Table 1). In another embodiment, a cathepsin S inhibitor of the invention is a thiocarbazate selected from the group of compounds consisting of Compound No. 13, 14, 17-20, 24, 29, 31, 34, 41, 44-54, 57, 58, 62, 63, 76, 77, and 82-84 (Table 1). In another embodiment, a cathepsin S inhibitor of the invention is an oxacarbazate selected from the group of compounds consisting of Compound No. 93-112 (Table 2). In another embodiment a cathepsin S inhibitor of the invention is an oxacarbazate selected from the group of compounds consisting of Compound No. 93, and 96 (Table 2). In yet another embodiment, a cathepsin S inhibitor of the invention is a diacyl hydrazine selected from the group of compounds consisting of Compound No. 113-117 (Table 3). In yet another embodiment, a cathepsin S inhibitor of the invention is an acyl hydrazine selected from the group of compounds consisting of Compound No. 118-119 (Table 4). In yet another embodiment, a cathepsin S inhibitor of the invention is an N-hydroxy-amide consisting of Compound No. 120 (Table 5). In yet another embodiment, a cathepsin S inhibitor of the invention is a dialdehyde consisting of Compound No. 121 (Table 6). In yet another embodiment, a cathepsin S inhibitor of the invention is a sulfonylated acyl hydrazine consisting of Compound No. 122 (Table 7). In yet another embodiment, a cathepsin S inhibitor of the invention is a acyl hydrazone selected from the group of compounds consisting of Compound No. 123-124 (Table 8). In yet another embodiment, a cathepsin S inhibitor of the invention is a acyl hydrazine carboxamide consisting of Compound No. 125 (Table 9). In yet another embodiment, a cathepsin S inhibitor of the invention is a acyl hydrazine carbodithioate consisting of Compound No. 126 (Table 10). In yet another embodiment, a cathepsin S inhibitor of the invention is a acyl hydrazine oxoacetamide consisting of Compound No. 127 (Table 11).

An autoimmune disease arises through aberrant reactions of the human adaptive or innate immune systems where a patient's immune system is activated against the body's own proteins. Examples of an autoimmune disease include, but are not limited to, psoriasis, rheumatoid arthritis, multiple sclerosis, and asthma.

In another embodiment, the invention provides a method of treating a subject at risk of developing an autoimmune disease, the method comprising prophylactically administering a therapeutically effective amount of at least one cathepsin S inhibitor to a subject in need thereof, where the cathepsin S inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof. In one embodiment, a cathepsin S inhibitor of the invention is a thiocarbazate selected from the group of compounds consisting of Compound No. 1 and 5-92 (Table 1). In another embodiment, a cathepsin S inhibitor of the invention is a thiocarbazate selected from the group of compounds consisting of Compound No. 13, 14, 17-20, 24, 29, 31, 34, 41, 44-54, 57, 58, 62, 63, 76, 77, and 82-84 (Table 1). In another embodiment, a cathepsin S inhibitor of the invention is an oxacarbazate selected from the group of compounds consisting of Compound No. 93-112 (Table 2). In another embodiment a cathepsin S inhibitor of the invention is an oxacarbazate selected from the group of compounds consisting of Compound No. 93, and 96 (Table 2). In yet another embodiment, a cathepsin S inhibitor of the invention is a diacyl hydrazine selected from the group of compounds consisting of Compound No. 113-117 (Table 3). In yet another embodiment, a cathepsin S inhibitor of the invention is an acyl hydrazine selected from the group of compounds consisting of Compound No. 118-119 (Table 4). In yet another embodiment, a cathepsin S inhibitor of the invention is an N-hydroxy-amide consisting of Compound No. 120 (Table 5). In yet another embodiment, a cathepsin S inhibitor of the invention is a dialdehyde consisting of Compound No. 121 (Table 6). In yet another embodiment, a cathepsin S inhibitor of the invention is a sulfonylated acyl hydrazine consisting of Compound No. 122 (Table 7). In yet another embodiment, a cathepsin S inhibitor of the invention is a acyl hydrazone selected from the group of compounds consisting of Compound No. 123-124 (Table 8). In yet another embodiment, a cathepsin S inhibitor of the invention is a acyl hydrazine carboxamide consisting of Compound No. 125 (Table 9). In yet another embodiment, a cathepsin S inhibitor of the invention is a acyl hydrazine carbodithioate consisting of Compound No. 126 (Table 10). In yet another embodiment, a cathepsin S inhibitor of the invention is a acyl hydrazine oxoacetamide consisting of Compound No. 127 (Table 11).

Although the methods of the invention require the administration of at least one cathepsin S inhibitor, two, three, four, five, six, seven, eight, nine, ten, or more cathepsin S inhibitors may also be used. Further, the skilled artisan will appreciate that the methods of the invention include administering at least one cathepsin S inhibitor in combination with at least one cathepsin L inhibitor, at least one cathepsin B inhibitor, or any combination thereof.

The invention may be practiced in any subject diagnosed with, or at risk of developing, an autoimmune disease, including but not limited to psoriasis, rheumatoid arthritis, multiple sclerosis, and asthma that relies on Cathepsin S expression or activity to maintain its pathology. Preferably, the subject is a mammal and more preferably, a human.

The methods of the present invention can be used in combination with other treatment regimens, including virostatic and virotoxic agents, antibiotic agents, antifungal agents, anti-inflammatory agents (steroidal and non-steroidal), antidepressants, anxiolytics, pain management agents, (acetaminophen, aspirin, ibuprofen, opiates (including morphine, hydrocodone, codeine, fentanyl, methadone), steroids (including prednisone and dexamethasone), and antidepressants (including gabapentin, amitriptyline, imipramine, doxepin) antihistamines, antitussives, muscle relaxants, brondhodilaters, beta-agonists, anticholinergics, corticosteroids, mast cell stabilizers, interferons, cytokines, and other immune modulators, leukotriene modifiers, methylxanthines, human immunoglobulins, nucleic acid based therapeutic agents, as well as combination therapies, and the like. The compounds of the present invention may be administered before, during, after, or throughout administration of any therapeutic agents used in the treatment of a subject's disease or disorder.

The invention can also be used in combination with other treatment modalities. These may include intensive supportive care such as assisted ventilation, intravenous and/or oral fluids, transfusion, and the like.

As envisioned in the present invention with respect to the disclosed compositions of matter and methods, in one aspect the embodiments of the invention comprise the components and/or steps disclosed therein. In another aspect, the embodiments of the invention consist essentially of the components and/or steps disclosed therein. In yet another aspect, the embodiments of the invention consist of the components and/or steps disclosed therein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

57,821 compounds from the NIH Molecular Libraries Small Molecule Repository (MLSMR) were screened against human liver cathepsin L. This screen was performed in 384-well Corning 3676 black, low-volume, non-binding surface (NBS)-coated polystyrene plates using a total reaction volume of 10 μL per well. Final well concentrations were: 10 μM compound (2% DMSO), 300 pM (8.7 ng/mL) human liver cathepsin L, 1 μM Z-FR-AMC substrate, and 20 mM sodium acetate buffer containing 1 mM EDTA and 5 mM DTT, pH 5.5. Cathepsin L was activated in the assay buffer for 30 minutes prior to dispensing into wells. Assay plates were incubated at room temperature for one how and the fluorescence intensity of each well was read with a PerkinElmer Envision plate reader (Ex: 355 nm, Em: 460 nm) to measure hydrolysis of the AMC substrate. The screen correctly identified E-64 and E-64c members of the library as potent inhibitors of cathepsin L. A Z′ factor of 0.73 was calculated for this screen, indicating good plate uniformity throughout the run.

From the 57,821 compound primary HTS, 102 compounds (0.18%) showed >45% inhibition against human cathepsin L. Upon a confirmatory dose response assay, 49 (48%) of these small molecules inhibited human cathepsin L activity with an IC₅₀<50 μM. Library samples containing 2,5-disubstituted oxadiazoles were identified as potent hits in a high throughput screen of the NIH Molecular Libraries Small Molecule Repository directed at discovering inhibitors of cathepsin L (Table I). However, when synthesized in pure form, the putative actives were found to be devoid of biological activity. Analyses by LC-MS of original library samples indicated the presence of a number of impurities, in addition to the oxadiazoles. Synthesis and bioassay of the probable impurities led to the identification of a thiocarbazate that likely originated via ring opening of the oxadiazole. Previously unknown, thiocarbazates were independently synthesized as single enantiomers and found to inhibit cathepsin L in the low nanomolar range.

### Cathepsin Assays Optimization

The cathepsin L assay was run with 1 μM Z-Phe-Arg-7-amido-4-methylcoumarin (Z-Phe-Arg-AMC, Sigma C9521) and 8.7 ng/mL human liver cathepsin L (Calbiochem 219402) in 100 μL reactions (96-well plate). Assay buffer consisted of 20 mM sodium acetate, 1 mM ethylenediaminetetraacetic acid (EDTA) and 5 mM cysteine, pH 5.5. Cathepsin L was incubated in assay buffer for 30 minutes prior to dispensing into wells to allow for efficient reduction of the active site cysteine required for full enzymatic activity. AMC dilution controls were performed and no inner filter effect quenching was observed at fluorophore concentrations as high as 50 μM.

Human spleen cathepsin S (Calbiochem 219344, 40 ng/mL) was assayed using 15 microM Z-Phe-Arg-AMC substrate. Human liver cathepsin B (Calbiochem 219362, 65 ng/mL) was assayed using 15 microM Z-Arg-Arg-AMC substrate (Bachem I-1135). All reactions were performed in 20 mM sodium acetate buffer containing 1 mM EDTA and 5 mM cysteine, pH 5.5.

### IC₅₀ Determination

IC₅₀ determinations were conducted with the following assay buffer: 20 mM sodium acetate, 1 mM EDTA, and 5 mM cysteine, pH 5.5. Compounds were serially diluted in DMSO and transferred into a 96-well Corning 3686 assay microplate to give a 16-point two-fold serial dilution dose response ranging from 25 microM to 760 pM. Human liver cathepsin L (Calbiochem 219402) was activated by incubating with assay buffer for 30 min. Upon activation, cathepsin L (300 pM, 8.7 ng/mL) was incubated with 1 microM Z-Phe-Arg-AMC substrate (Sigma C9521) and test compound in 100 mL of assay buffer for 1 hour at room temperature. Fluorescence of AMC released by enzyme-catalyzed hydrolysis of Z-Phe-Arg-AMC was read on a PerkinElmer Envision microplate reader (excitation 355 nm, emission 460 nm). Data were scaled using internal controls and fitted to a four-parameter logistic model (IDBS XLfit equation 205) to obtain IC₅₀ values in triplicate.

### Preincubation Studies

To establish the time-dependent mechanism of inhibition, enzyme and inhibitor were preincubated for various time points in a 96-well microplate prior to the addition of substrate to initiate the enzymatic reaction. 47.5 μL of cathepsin L (18.3 ng/mL) and 47.5 μL of Compound No. 1 at various concentrations in assay buffer were incubated up to 4 hours. Five μL of Z-Phe-Arg-AMC substrate were then added and the plate was monitored for AMC hydrolysis on the Envision fluorescent microplate reader.

### Reversibility

To test the reversibility of Compound No. 1, cathepsin L at 100-fold its final assay concentration (870 ng/mL) and inhibitor at 10-fold its IC₅₀ after 1 hour preincubation were combined and incubated for 1 hour at room temperature at 2 μL. This mixture was then diluted 100-fold in a Corning 3650 96-well plate with assay buffer containing 1 μM Z-Phe-Arg-AMC to a final volume of 200 μL. A rapidly reversible inhibitor should dissociate from the enzyme to restore approximately >90% of enzymatic activity. Fluorescence intensities of the 200 μL reaction wells were monitored continuously for AMC hydrolysis on the Envision plate reader.

### Data Fitting

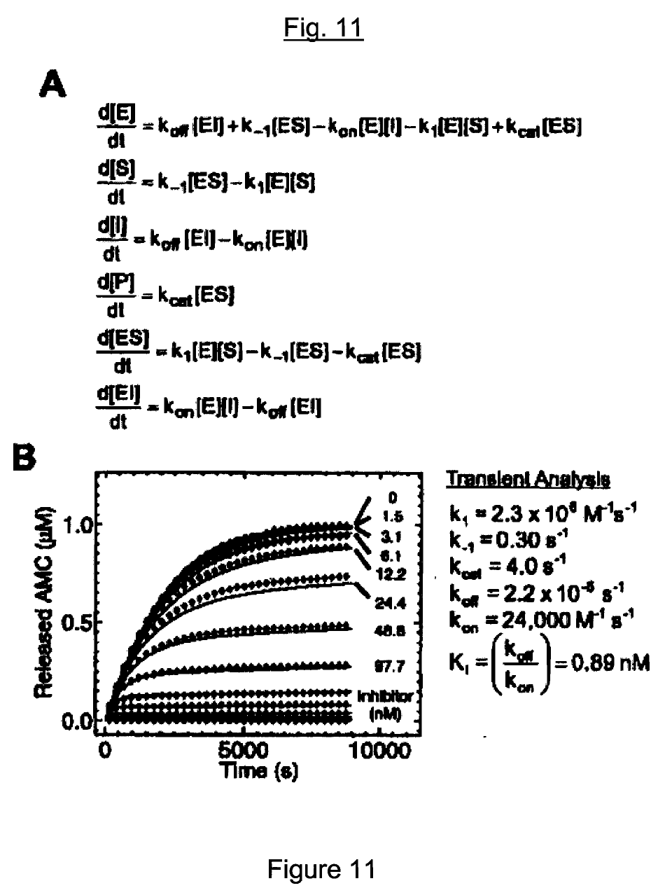

In the kinetic simulations, the concentrations of chemical species ([E], [S], [I], [P], [ES], [EI]) over time were calculated using a system of ordinary differential equations for each reaction step (FIG. 11A). Progress curves for each inhibitor concentration were fit to a five-parameter (k₁, k₋₁, kₒₙ, k_{off}, k_{cat}) kinetic inhibition model using APPSPACK optimization software. APPSPACK is a generic solver for linearly-constrained optimization problems (Griffin and Kolda, 2006, Asynchronous Parallel Generating Set Search for Linearity: Constrained Optimization, Sandia National Laboratories, Livermore, Calif.).

### Selectivity

Compound No. 1 was assayed for inhibition against papain and cathepsins B, G, K, S, and V. Papain from Carica papaya (Calbiochem 5125, 11 ng/mL), human cathepsin K (Calbiochem 342001, 35 ng/mL), human spleen cathepsin S (Calbiochem 219344, 40 ng/mL), and human cathepsin V (Calbiochem 219467, 39 ng/ml) were assayed using Z-Phe-Arg-AMC substrate at 20 μM, 20 μM, 15 μM, and 1 μM, respectively. Human liver cathepsin B (Calbiochem 219362, 65 ng/mL) was assayed using 15 μM Z-Arg-Arg-AMC substrate (Bachem I-1135). Human neutrophil cathepsin G (Calbiochem 219373, 4.2 μg/mL) was assayed using 15 μM Suc-Ala-Ala-Pro-Phe-AMC substrate (Sigma S9761). All reactions were performed in 20 mM sodium acetate buffer containing 5 mM cysteine and 1 mM EDTA, pH 5.5. Reaction progress was monitored using the Envision microplate reader. IC₅₀ values were measured in triplicate.

### Cytotoxicity

Human aortic endothelial cells were seeded in a Corning 3704 384-well white sterile tissue culture-treated microplate at 1000 cells/μL/well. The plate was centrifuged and incubated at 37° C. for 24 hr. Compound No. 1 and doxorubicin positive control were then serially diluted in EGM-2 endothelial cell media (Lonza CC4176). Five μL each of these serial dilutions were added to the cells in triplicate, resulting in final concentrations of compound from either 100 pM to 156 nM or 100 μM to 156 nM (0.17% DMSO). The plate was centrifuged and incubated at 37° C. for 24 hrs.

Thirty μL CellTiter-Glo™ (Promega G7570) were added to each well and centrifuged. After 10 minutes, luminescence was measured using the Envision microplate reader.

### Malaria Assay

Eight two-fold serial dilutions of Compound No. 1 in RPMI 1640 media (Invitrogen 1 1 875) containing L-Glutamine, 50 mg/L hypoxanthine, 6 g/L HEPES, 0.5% Albumax 11 bovine serum (Invitrogen 11021), 0.225% sodium bicarbonate, and 1 pgIrnL gentamicin were performed in Corning 3704 microplates. Adding 30 μL red blood cells infected with synchronized ring stage luciferase-expressing Plasmodium falciparum parasites at 0.5% parasitaemia and 4% hematocrit to 10 μL compound resulted in final concentrations tested of 50 μM to 1.5 nM. In addition, 30 μL normal red blood cells and 30 μL infected red blood cells were added to two control columns containing 10 μL media. The plates were incubated at 37° C. in a 92% humidity chamber with 5% CO₂, 5% O₂, and 90% N₂ for 48 hours to allow for two cycles of red blood cell rupture and invasion to take place. Forty μL BrightGlo™ (Promega E2610) were added to each well and centrifuged. After 5 minutes, luminescence was measured using the Envision microplate reader.

### Leishmaniasis Assay

Five thousand Leishmania major promastigotes were plated per well in a 384-well microtiter plate in a 20 μL volume of promastigote growth medium. Promastigotes were treated with a concentration range from 0 μM to 50 μM of Compound No. 1 for 44 hrs. Five pL Cell-Titer-Blue™ were added per well and incubated for 4 hrs. Relative fluorescence units (A₅₆₀/A₅₉₀) were captured on a SpectraMax M5 microtiter plate reader. DMSO concentrations were held constant at 0.5%.

The results of the experiments presented in the Examples are now described.

### Example 1

### Design of the Thiocarbazate Library

The strategy involved the design of a library containing a thiocarbazate scaffold incorporating a variety of functional groups at three different positions A, B and C (FIG. 1). From the outset, optimal diversity of the final products was sought in terms of size, shape and functionality. At the same time, optimal physical properties were maintained to ensure solubility, permeability and other “drug-like” properties. Finally, a strict requirement was adhered to for an expedient synthesis that would produce a minimum of 10 mg of final product in purities of at least 95% as determined by LC/MS analysis. Modifications at the A position involved changes in size, as well as replacement of the t-butyloxycarbonyl group. Modifications made at this position addressed the issue of instability when a free amine is present at this position. Position B underwent the most extensive modifications, where changes in size, polarity, acidity, and functionality were incorporated. Thiocarbazates derived from natural amino acids, such as methionine, valine, alanine, glutamic acid, leucine, proline, phenylalanine, tyrosine, threonine, serine, glutamic acid, lysine, arginine and histidine, along with unnatural amino acids were prepared. Modifications at C involved incorporation of ring constraints, removal of the amide bond and exploration of size requirements; a variety of acetamides derived from aniline, primary amines, and methyl esters were included. Examples of substituents at position C include differentially substituted anilines, quinolines and isoquinolines, non-aromatic amines, morpholines, indoline, and pyridinone.

To ensure that the library contained compounds with a range of acceptable physical properties such as logP, molecular weight, polar surface area etc, cheminformatics tool, Leadscope (Leadscope, Inc.) was relied upon. Table 12 below details this analysis. The aggregated library exhibited excellent properties according to Lipinski-like parameters (Lipinski et al., 2001, Adv. Drug Del. Rev. 46:3): average Log P=3.5, average H-bond acceptors=4.8, average H-bond donors=3.9. The average molecular weight (526) is typical of small molecule protease inhibitors, but slightly above the Lipinski target of <500. Consequently, the number of Lipinski violations on average was 1, which is reflective of the molecular weight characteristics. Polar surface area and rotable bonds were on average 134 A and 16, respectively. While the PSA was within the range suggested by Veber et al., 2002, (J. Med. Chem. 45:2615) for orally available drugs, the rotatable bond count was somewhat higher than suggested as optimal.

TABLE 12
Physical properties of thiocarbazate library.

| | Average | Range | Median | Mode |
|---|---|---|---|---|
| AlogP | 3.5 | 1.0-6.0 | 3.5 | 4.1 |
| H-Bond Acceptors | 4.8 | 3-7 | 5.0 | 5.0 |
| H-Bond Donors | 3.9 | 2-7 | 4.0 | 4.0 |
| Lipinski Violations | 1.0 | 0-3 | 1.0 | 1.0 |
| Molecular Weight | 526.1 | 367.2-706.6 | 536.6 | 539.6 |
| Polar Surface Area | 134.3 | 95.5-210.8 | 132.6 | 125.6 |
| Rotatable Bonds | 16 | 9-23 | 16 | 16 |

### Example 2

### Synthesis of the Thiocarbazate Library

Based on chemistry described in Myers et al, 2008, (Bioorg. Med. Chem. Lett. 18:210) and Myers et al., 2008, (Bioorg. Med. Chem. Lett. 18:3646), a versatile synthetic strategy was designed to prepare the thiocarbazate library as illustrated in previously shown Scheme 2. A variety of acids were treated with ethylchloroformate to form the corresponding mixed anhydride, which were not isolated. Treatment with hydrazine monohydrate then furnished the hydrazides which were isolated after aqueous workup. Reaction with carbonyl sulfide gas in ethanol (Chande et al., 1998, Indian J. Chem. 37B, 352) formed the thiosemicarbazide intermediates that were directly treated with an alkylating agent to form the thiocarbazate products. The thiocarbazates were isolated and purified by HPLC to at least 95% purity. All compounds were characterized by high resolution mass spectroscopy, and a subset further characterized by ¹H NMR, ¹³C NMR and IR. The general procedure for the preparation of thiocarbazates was amenable to all of the thiocarbazates produced in the library (Table 1).

While many of the amino acid and acid starting materials were commercially available, several required preparation. Hydrazides derived from commercially available R and S-2-methyl-3-hydroxypropionate were generated directly from the ester as shown in Scheme 3, then converted to the corresponding thiocarbazates (e.g., thiocarbazates of formulas 67-70). Subsequent etherification of formula 69 afforded the butyldimethylsilyl (TBS) (Patrick et al., 2004, Org. Biomol. Chem. 2:2220), and para-methoxyl benzyl (PMB) (Hayashi et al., 2007, J. Am. Chem. Soc. 129:12650) ether analogs 71 and 72. Beta amino acids, such as those incorporated into thiocarbazates of formulas 73-81, were prepared using a modified Arndt-Eistert protocol to furnish the desired α-amino acid in yields of 70-95% (Linder et al., 2002, Org. Synth. 79:154).

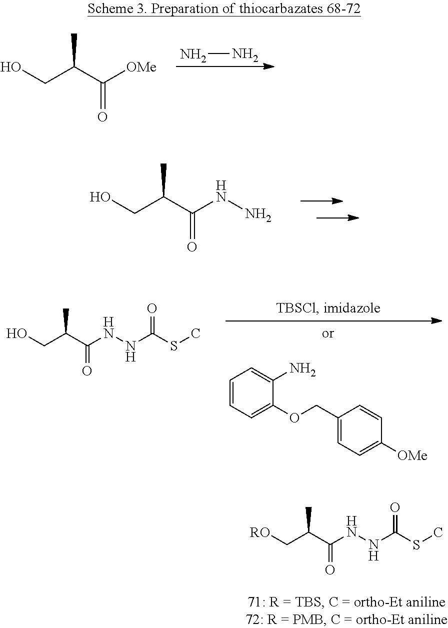

### Example 3

### Characterization of the Thiocarbazate Library

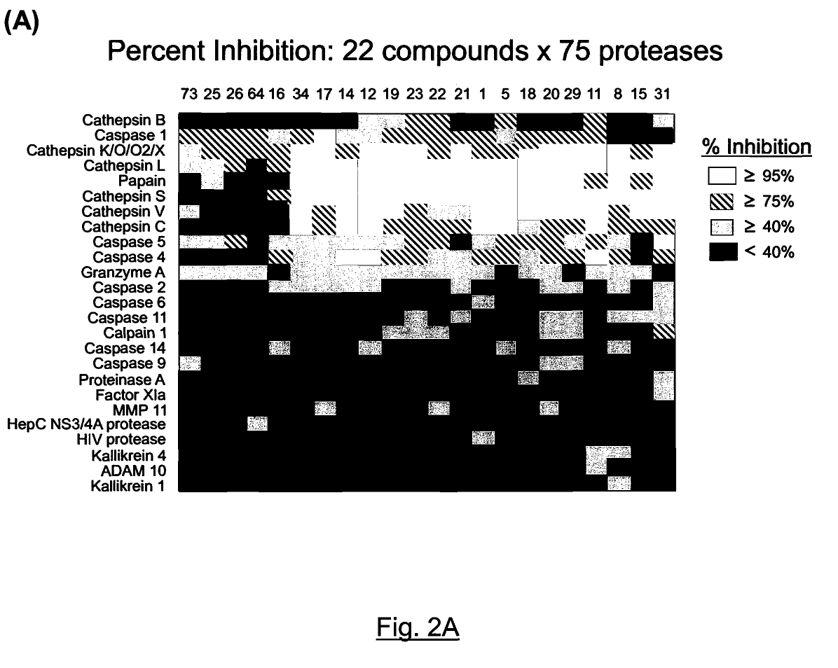

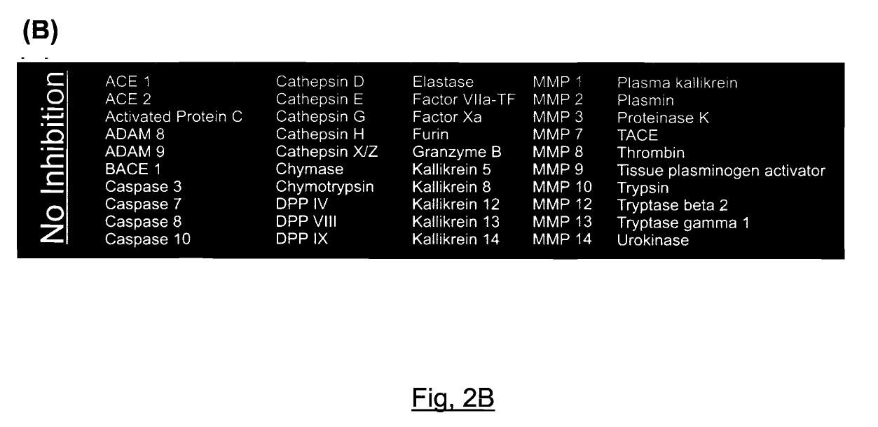

Thiocarbazates and their activity as protease inhibitors have not been described previously. A subset of twenty-two compounds was profiled at a concentration of 10 μM for inhibitory activity against 75 different proteases (www.reactionbiology.com) (FIG. 2). The proteases chosen covered a broad spectrum of classes including serine proteases, metalloproteases, aspartyl proteases and cysteine proteases. The aim of this study was to determine quickly whether thiocarbazates as a class displayed selectivity towards different families of proteases, or displayed broad protease inhibition properties.

The heatmap illustrated in FIG. 2 illustrates several broad conclusions. First, the thiocarbazates as a class exhibit selectivity towards cysteine proteases. No significant activity was detected against any other protease family member (e.g. serine, aspartyl, and metalloproteases). Second, even among the cysteine protease family, a preference for the papain family is observed, as only modest activity against representatives of the calpain or caspase families is observed. Finally, several thiocarbazates exhibit potent activity (>95% inhibition at 10 μM) against Cathepsins L, S, V, K and papain.

Further attention was focused on a more thorough biological characterization of this library against Cathepsins B, L and S. This choice was based on the potential for potent inhibition, as well as selectivity. In addition, all three cathepsins represent important targets for drug discovery efforts. Towards this end, IC₅₀'s were generated for all 82 members of the thiocarbazate library (Table 1) as well as the other chemotype compounds (Tables 2-11) against Cathepsins B, L and S.

Based on these data, several generalizations for broad cathepsin inhibition are apparent. First, the substitution patterns displayed at positions A and B are key determinants for inhibitory activity. Alpha amino acid derived thiocarbazates are the preferred substituents, while those prepared from beta amino acid derivatives (thiocarbazates of Formula No. 74-81, Table 1) and acid precursors (thiocarbazates of formulas 67-73, Table 1) are devoid of activity. Among the thiocarbazates that incorporate alpha amino acids, the size of substituent B had a profound effect on potency. Thiocarbazates containing large groups (such as 3-indolemethylene, benzyl, 4-benzyloxybenzyl) or medium sized substituents (such as isopropyl, methyl thioethyl) were more potent than those incorporating smaller groups [e.g., hydrogen (thiocarbazate of Formula No. 64, Table 1), methyl (thiocarbazate of Formula No. 25, Table 1).] Incorporation of the specific amino acids proline (thiocarbazates of Formula No. 32, 33, 80, 81, Table 1), histidine (thiocarbazates of Formula No. 65, 66, Table 1) or glutamic acid (thiocarbazates of Formula No. 27, 28, 59, 60, 61, Table 1) proved detrimental to activity. Stereochemistry preferences at position B were also explored through the preparation a key enantiomeric pairs such as Formula No. 4 and 16; 46 and 48; and 47 and 49 of Table 1. In those examples, the thiocarbazate derived from the L-amino acid was more potent than that derived from the D isomer. Structural requirements for activity at position C involved a strong preference for a carbonyl group (Formula No. 6 vs. 4; 30 vs. 29; 35 vs. 34, Table 1). In all cases where the direct comparison could be made, removal of the carbonyl group significantly diminishes the inhibitory activity against all three cathepsins. In contrast, the specific nature of the amine side chain influenced activity far less.

### Example 4

### Identification and Synthesis of a Unique Thiocarbazate Cathepsin L Inhibitor

The Penn Center for Molecular Discovery (PCMD) recently completed a high throughput screening (HTS) campaign of the NIH Molecular Libraries Small Molecule Repository (MLSMR) to identify inhibitors of members of the papain-like cysteine protease family, including cathepsins B, L, and S (Myers et al., 2007, Bioorg. Med. Chem. Lett. 17:4761).

Previously reported inhibitors of cathepsin L include the peptides, leupeptin and aprotinin, and the fluoromethyl ketone, Z-LLL-FMK. The few known-potent small molecule inhibitors are either peptidic and therefore suffer from physiological instability and poor permeability, or are non-selective for cathepsin L (Esser et al., 1994, Arthritis Rheum. 37-236; Montaser et al., 2002, J. Biol. Chem. 383:1305; Fujishima et al., 1997, FEBS Lett. 407:47). The identification of potent, selective, stable, and cell permeable small-molecule inhibitors would therefore provide valuable tools to interrogate cathepsin L and cathepsin L-like function, as well as potential starting points for drug discovery and development.

TABLE 13
Cathepsin L inhibitory activity of
oxadiazole-containing mixtures library samples.^{a} 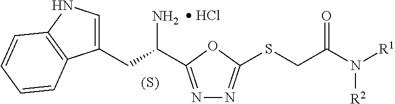
Impurities

| PubChem SID | R¹ | R² | IC50 (uM)^{b} |
|---|---|---|---|
| 861540 (1) | 2-ethylbenzene | H | 0.13 ± 0.01 |
| 861087 (2) | 2,4-dimethylbenzene | H | 0.16 ± 0.05 |
| 861840 (3) | Me | Me | 0.17 ± 0.02 |
| 861542 (4) | 2,3-dimethylbenzene | H | 0.30 ± 0.04 |
| 861992 (5) | Et | Et | 0.51 ± 0.02 |

^{a}Library samples containing the parent oxadiazole with impurities.
^{b}IC₅₀ values are reported as mean ± standard deviation (number of determinations = 3).

Initial HTS results of our cathepsin L screen indicated that several structurally compounds exhibited potent inhibitory activity. Library samples containing the putative active oxadiazoles were evaluated for both purity and integrity by LC-MS analysis. This analysis indicated that the primary constituent of each sample was indeed the expected oxadiazole, in up to 60% purity. However numerous impurities were also present. Table 13 presents apparent IC₅₀ data obtained for mixtures of compounds, the parent compound of which (the oxadiazole) is presented in Table 13. The carbazate contaminant that is created by the ring opening of the oxadiazole by water is the active compound responsible for the apparent IC₅₀˜100 nM activity presented in Table 13. The active carbazate contaminant is a smaller fraction of the mass in the mixture, roughly 10-30% based on LCMS. The most potent hit exhibited an IC₅₀ of 0.13 μM. To confirm the biological activity attributed to 1, a synthetic sequence was developed to generate the oxadiazoles in pure form which was found to be devoid of activity.

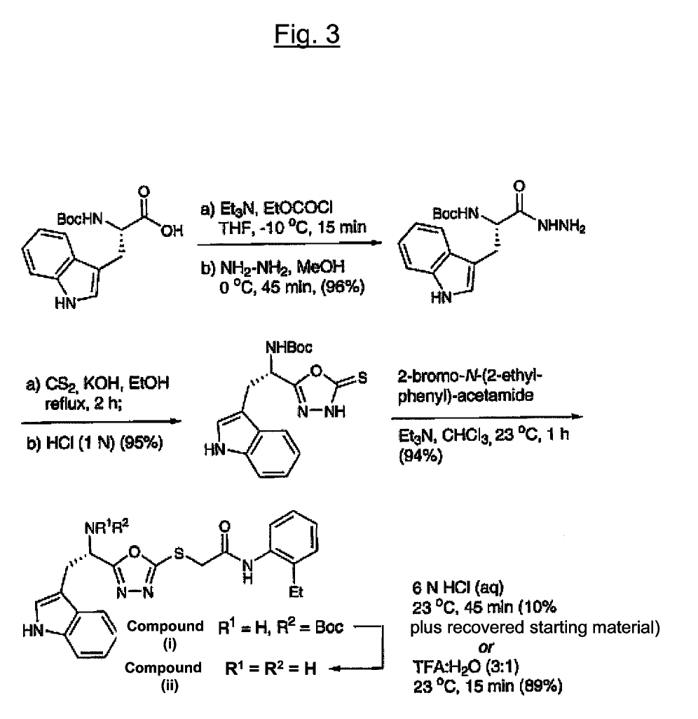

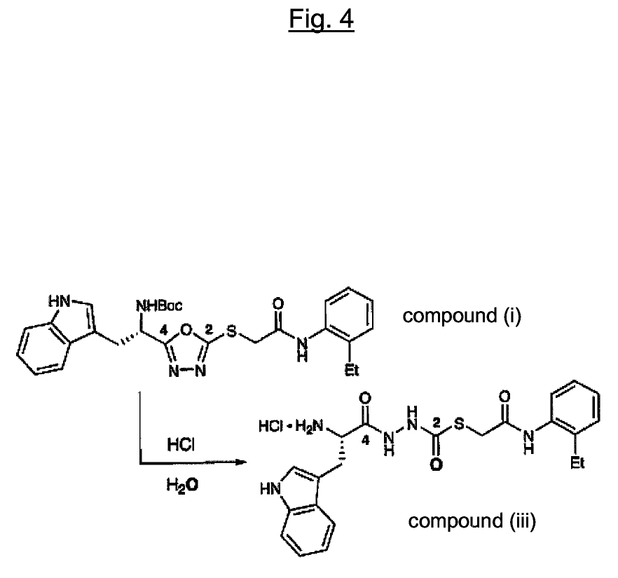

Although little literature precedence exists for the construction of compounds such as Compound (ii) (shown in FIG. 3), the present invention discloses modification of the Woodward-Confalone (Confalone and Woodwar, 1983, J. Am. Chem. Soc. 105:902) approach to α-amino acid substituted oxadiazolethiones could provide an entry to this class of sulfur-based 2,5-disubstituted oxadiazoles. Toward this end, commercially available L-Boc-Trp-OH was converted in high yield to the corresponding hydrazide, on a 10-g scale (FIG. 3). Cyclization with carbon disulfide in ethanol at reflux afforded the expected substituted thione (FIG. 3) as a stable solid in excellent yield, which upon chemoselective alkylation with 2-bromo-N-(2-ethyl-phenyl)-acetamide (FIG. 3) efficiently generated Compound (i) (FIG. 3; 94% yield). Further study indicated that isolation of the thione (FIG. 3) was unnecessary, and Compound (i) (FIG. 3 and FIG. 4) could be prepared directly from the hydrazide precursor (FIG. 3), in a single flask. Both sequences furnished Compound (i) (FIG. 3 and FIG. 4) in high yield. In similar fashion, the antipode of Compound (i) was prepared starting from D-Boc-Trp-OH. High enantiomeric purities (>99%) were demonstrated via chiral supercritical fluid chromatography.

Initial attempts to remove the Boc-group utilizing HCl in dioxane (4 N) or HCl in water (6 N) were compromised by the poor solubility of Compound (i) (FIG. 3 and FIG. 4). However, small quantities of the HCl salt of Compound (ii) (FIG. 3) could be obtained by treatment with 6 N HCl over 45 minutes. Upon LCMS analysis of synthetic Compound (ii) (FIG. 3), it was recognized that the impurities formed during the acid-promoted Boc-deprotection step were identical to those found in the library screening samples. Optimal conditions to remove the Boc-group were eventually developed. Specifically, treatment of Compound (i) (FIG. 3 and FIG. 4) with 25% water in TFA for 15 minutes, followed in turn by adjustment of the pH to 8.0 with NaHCO₃ and aqueous extraction with methylene chloride-furnished the free-base of Compound (ii) (FIG. 3) in 89% yield and 99% purity after column chromatography. In the end, oxadiazole Compound (ii) (FIG. 3) was prepared on gram scale from L-Boc-Trp-OH; the overall yield was 76%.

The free base of Compound (ii) was found to be completely devoid of activity when assayed against cathepsin L. This result suggested that an impurity present in the original library sample was responsible for the observed activity. Based on LC-MS analyses of the biologically active samples, it was hypothesized that the active component was likely the ring-opened product Compound (iii) (FIG. 4), formed via acid-promoted addition of H₂O to Compound (i) (FIG. 4). The presence of a molecular [M+1] ion equal to 440 amu in the LC-MS analysis of impure samples was consistent with the presence of Compound (iii), thereby providing strong support for the hypothesis.

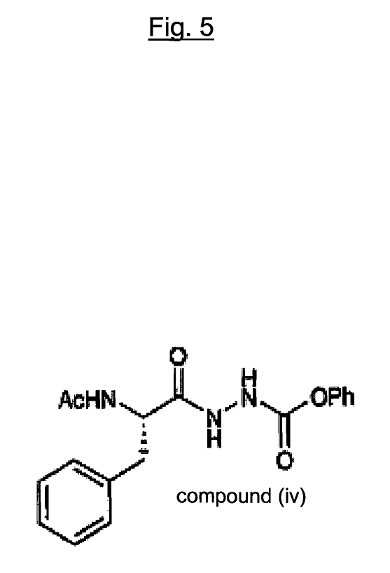

Thiocarbazates such as Compound (iii) (FIG. 4) have not been described previously in the literature. However, these compounds do bear structural resemblance to aza-peptides (e.g., Compound (iv) in FIG. 5), examples of which have been reported to exhibit cysteine protease inhibitory activity through a mechanism involving attack by the active site cysteine on the carbamate carbonyl.

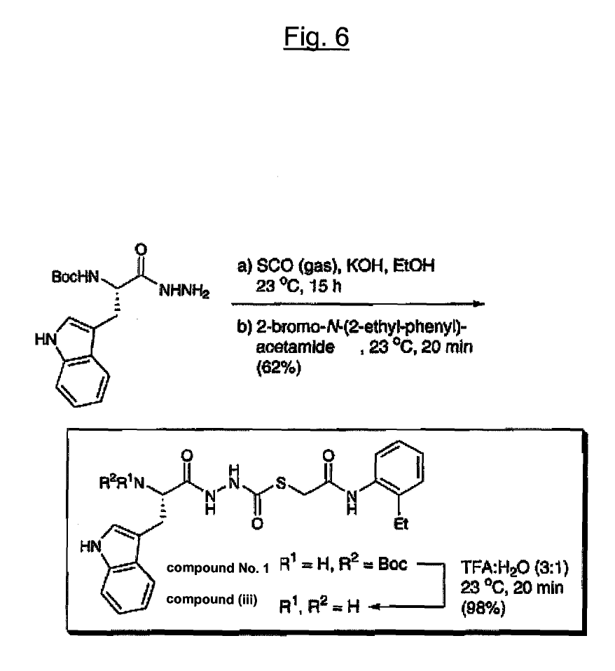

To test the hypothesis that Compound (iii) was indeed the active species, an expedient synthesis of this structural class was devised. It was hypothesized that introduction of the C2 carbonyl unit of Compound (iii) could be achieved via chemistry parallel to that used to prepare Compound (ii), beginning with the hydrazide shown in FIG. 6. The synthesis was begun by converting tryptophan hydrazide to an intermediate thiosemicarbazide (not shown) employing, carbonyl sulfide (S═C═O) gas dissolved in ethanol (FIG. 6). The intermediate thiosemicarbazide did not precipitate from solution, therefore 2-bromo-N-(2-ethyl-phenyl)-acetamide was added to the reaction flask to generate thiocarbazate Compound No. 1 (FIG. 6). The yield for the two-step, one-flask operation was 62%. Deprotection of Compound No. 1, employing the previously developed conditions (25% water in TFA), generated the free base Compound (iii) in 98% yield with >95% purity (FIG. 6). This three-step sequence permitted construction of Compound (iii) on gram scale, starting from L-Boc-Trp-OH; the overall yield was 58%.

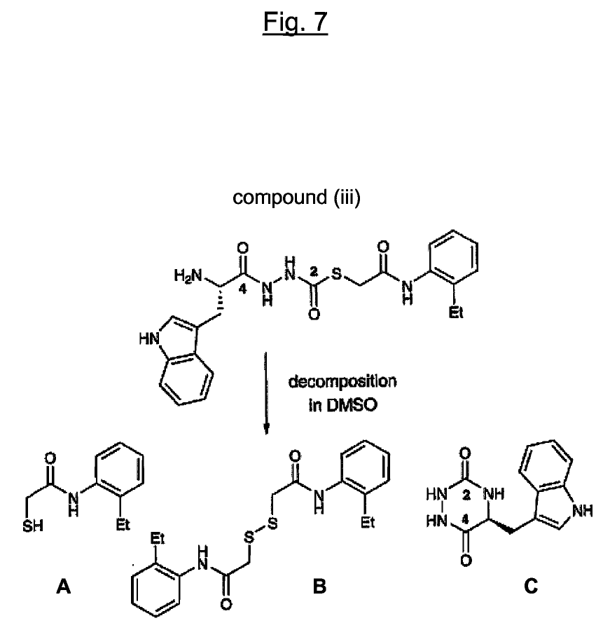

Both Compound No. 1 and Compound (iii) exhibited potent inhibitory activity against cathepsin L with IC₅₀ values of 56 nM and 133 nM, respectively. The R-enantiomer of Compound No. 1 was only modestly active against cathepsin L (IC₅₀=34 μM). Some variability in IC₅₀ values determined for Compound (iii) prompted us to explore the stability of both Compound No. 1 and Compound (iii) in solvents relevant to the bioassay. While Compound No. 1 was found to be completely stable in DMSO, as well as in the buffer employed in the cathepsin L assay [NaOAc (20 mM), pH 5.5; EDTA (1 mM); cysteine (5 mM)], the free base Compound (iii) proved unstable in DMSO, generating decomposition products A, B and C after only 1 h at room temperature (FIG. 7).

Presumably decomposition products A-C are formed from Compound (iii) via intramolecular attack of the primary amine on the C2 thiocarbazate moiety, with release of Compound A, FIG. 7. These products, prepared either via synthesis (Compounds A and B, FIG. 7) or by HPLC purification of decomposed material (Compound C, FIG. 7), were assayed for cathepsin L activity and found to be inactive. Due to the instability of Compound (iii) under the assay conditions, IC₅₀ values vary somewhat, rendering interpretation of the bioassay data difficult. The bioassay results for Compound No. 1 are accurate, as this compound is stable under all conditions evaluated.

Since most cysteine protease inhibitors contain an electrophilic ‘warhead,’ and work through a mechanism involving reaction with the active site cysteine, it is thought these novel thiocarbazates behave similarly, and are active by virtue of their electrophilic carbonyl moiety. The formation of Compound C (FIG. 7) supports this mechanism and is indicative of the electrophilicity of the C2 carbonyl group. Reactivity at the anilide carbonyl is also a possibility and cannot be ruled out. However, no evidence of side-products resulting from reaction at this position (FIG. 7) was observed.

In summary, samples from the NIH MLSMR revealed promising cathepsin L inhibitory activity attributed to a series of 2,5-disubstituted oxadiazoles. Analytical analyses (LC-MS) of the library samples, however, indicated numerous impurities, thus requiring the development of an efficient synthesis for the putative active 2,5-disubstituted oxadiazoles. Synthetic samples of pure 2,5-disubstituted oxadiazoles were found to be completely devoid of cathepsin L inhibitory activity. Careful LC-MS investigation of both the library and synthetic samples revealed a thiocarbazate to be the active component. Bioassay of the synthetic thiocarbazates confirmed the hypothesis: Compound A and Compound (iii) display potent inhibitory activity against cathepsin L, with IC₅₀ values of 56 nM and 133 nM, respectively, however instability of Compound (iii) was noted.

### Example 5

### Molecular Docking of Compound No. 1, a Novel, Potent and Selective Inhibitor of Human Cathepsin L

### Kinetic Characterization

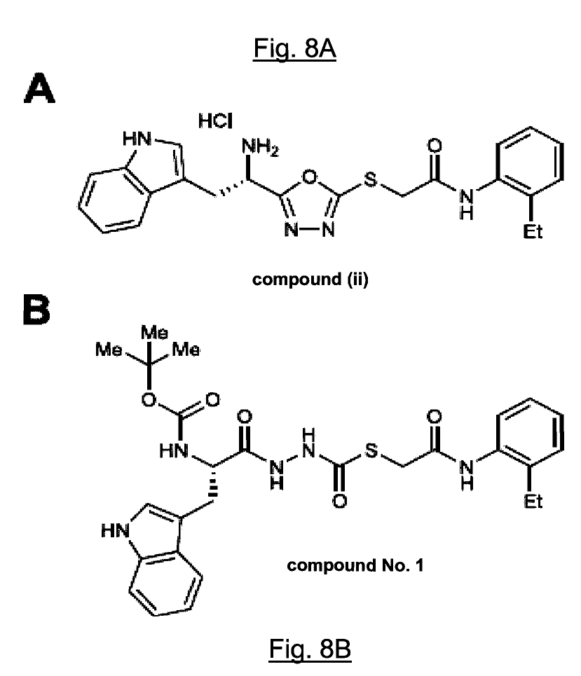

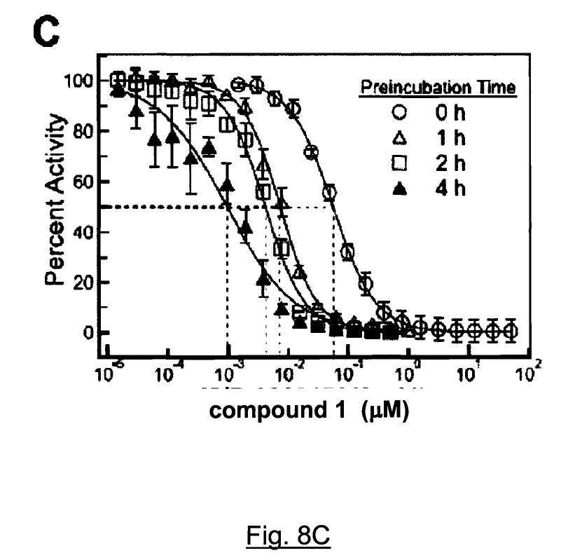

With immediate mixing of enzyme, substrate and inhibitor (no preincubation of enzyme and inhibitor), Compound No. 1 was found to inhibit human cathepsin L with an IC₅₀ of 56±4 nM. After preincubation with enzyme for 1, 2, and 4 hours prior to substrate addition at t=0, Compound No. 1 displayed increasing potency with IC₅₀ values falling to 7.5±1.0 nM, 4.2±0.6 nM, and 1.0±0.5 nM, respectively, demonstrating a slow onset of inhibition against the target enzyme (FIG. 8).

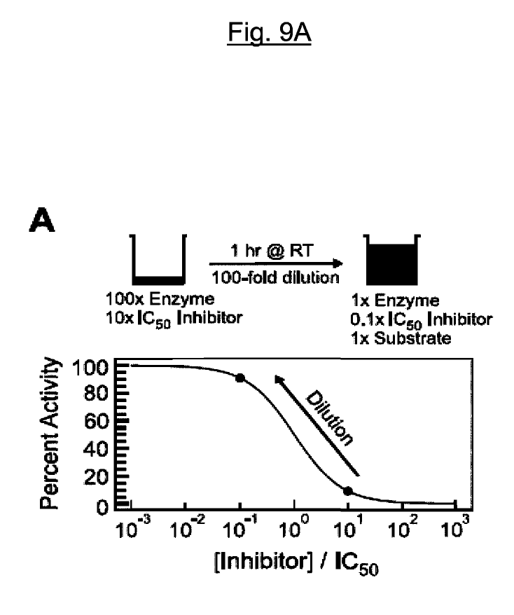

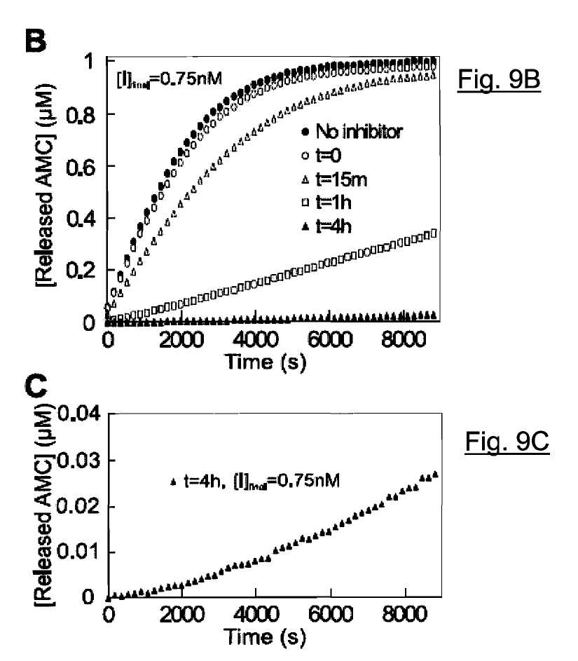

The mechanism of inhibition, to determine whether the compound acted as a rapidly reversible, slowly reversible, or irreversible inhibitor, was evaluated using a preincubation/dilution assay (Copeland, 2005, Evaluation of Enzyme Inhibitors in Drug Discovery: A Guide for Medicinal Chemists and Pharmacologists, J. Wiley, Hoboken, N.J.). By preincubating human cathepsin L and the compound for 1 hr at 10-fold its IC₅₀ after 1 hour preincubation (75 nM), a condition is created whereby >90% of the enzyme should be in an enzyme-inhibitor complex (FIG. 9A). Upon 100-fold dilution of the 1 hour preincubated mixture of cathepsin L and the inhibitor into assay buffer containing 1 pM ZPhe-Arg-AMC substrate, approximately 11% enzymatic activity was returned after 6000 seconds (s) into the reaction, by comparison of the substrate conversion rates of the preincubated and uninhibited reactions (FIG. 9B). For the 4 hr preincubated enzyme-inhibitor reaction condition (FIG. 9C), 99.8% of the reaction was inhibited immediately after addition of substrate due to almost all the enzyme being bound to small molecule inhibitor Compound No. 1. After 8820 s, the rate of product formation for the 4 hour preincubated reaction was 4.7 times greater than the initial rate of product formation, showing that the inhibitor was being released from the enzyme-inhibitor complex and enzymatic activity was indeed recovering. Therefore, Compound No. 1 was determined to be a very slowly reversible inhibitor of human cathepsin L.

### Nonlinear Regression of Transient Kinetics

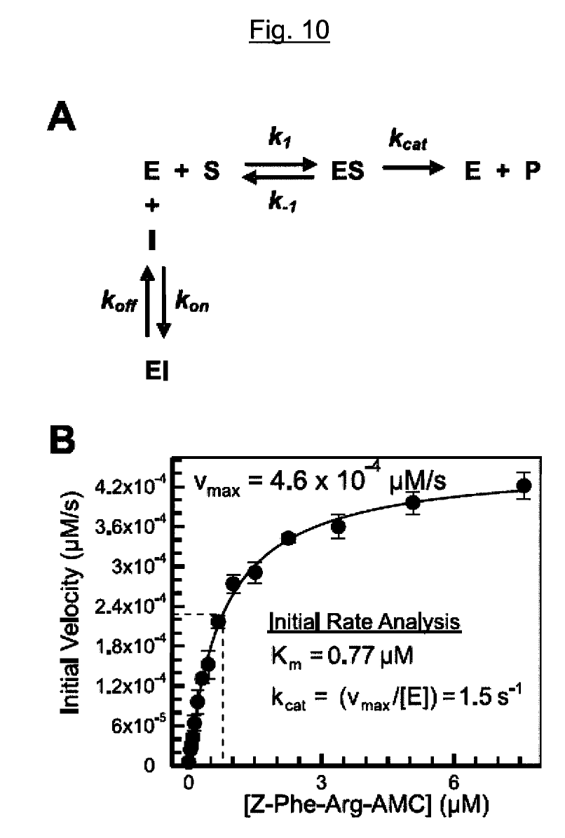

For human cathepsin L cleavage of Z-Phe-Arg-AMC, Kₘ and k_{cat} were determined through initial rate analysis to be 0.77 μM and 1.5 s⁻¹, respectively (FIG. 10B). A nonlinear regression for transient dynamics was conducted based on the reaction scheme shown in FIG. 10A. Here, the values of k₁, k₋₁, kₒₙ, and k_{off} are explicitly estimated rather than combined into the equilibrium parameters, Kₘ and Kᵢ, estimated by traditional kinetic analyses. The best fit parameters were k₁=2.3×10⁶ M⁻¹s⁻¹, k₋₁=0.30 s⁻¹, k_{cat}=4.0 s⁻¹, kₒₙ=24,000 M⁻¹s⁻¹, and k_{off}=2.2×10⁻⁵ s⁻¹ (FIG. 11B). The regressed Kᵢ=0.89 nM was quite consistent with the measured IC₅₀=1.0±0.5 nM obtained after 4 hr preincubation of human cathepsin L with SID 26681509. To explore alternate models for inhibition, the data were fit to models for irreversible inhibitor binding ([E]+[I]→[EI]); two-step inhibitor binding ([E]+[I] [symbol] [EI]₁ [symbol] [EI]₂), where a weak enzyme-inhibitor encounter complex is formed prior to the formation of a more tightly-bound enzyme-inhibitor complex; and uncompetitive inhibitor binding ([ES]+[I] [symbol] [ESI]), where inhibitor binds only to the enzyme-substrate complex. These models failed to reproduce the data as well as the five-parameter model described above for reversible, single-step competitive inhibition.

### Mechanism of Reversibility

The return of activity shown in FIG. 9C demonstrated that the thiocarbazate was a reversible inhibitor. Transient kinetic analyses (FIG. 11) quantified the rate of reversibility. To investigate the mechanism of reversibility and the generation of a putative leaving group, a stoichiometric reaction between 4.5 μM cathepsin L and 4.5 μM Compound No. 1 was analyzed by liquid chromatography-mass spectrometry [Shimadzu LC-MS/4.6 mm×50 mm Premier C18 column, 1 mL/min and a step from 90:10 to 60:40 water:acetonitrile with 10 min hold time, mobile phase contained 0.05% formic acid]. A potential thiol leaving group formed by reaction of cathepsin L with the thiocarbazate carbonyl of Compound No. 1 was synthesized (MW=195) and was detectable on the LC-MS at a concentration of 100 nM with a

retention time of 12.1 minutes (no suppression detected due to presence of human cathepsin L). However, this thiol leaving group was not detected by LC-MS after 6, 12, and 24 hours incubation of human cathepsin L with Compound No. 1. While this result argues against acylation of cathepsin by the inhibitor, formation of a tetrahedral intermediate by attack of the active site Cys residue on the thiocarbazate carbonyl of Compound No. 1 is not excluded. In fact, when the thiocarbazate sulfur in Compound No. 1 is replaced by carbon, the resulting molecule is a much weaker inhibitor (IC₅₀>50 μM, data not shown).

### Selectivity Against Papain and Cathepsins B, G, K, S, and V

Compound No. 1 was tested for inhibitory activity against papain and human cathepsins B, G, K, L, S, and V (Table 14) with no preincubation of enzyme and inhibitor. IC₅₀ values were calculated at time points of 10, 30, 60, and 90 minutes. The selectivity indexes of Compound No. 1 (a ratio of the IC₅₀ against the assayed protease divided by the IC₅₀ against cathepsin L) ranged from 7 to 151 for the various papain-like cysteine proteases (Table 14). Compound No. 1 inhibited papain and cathepsins B, K, S, and V with IC₅₀ values determined after one hour ranging from 618 nM to 8.442 μM. As expected, Compound No. 1 showed no inhibitory activity against the serine protease cathepsin G.

The IC₅₀ values systematically decreased with time for each protease, demonstrating the slow binding nature of the small molecule inhibitor. The qualitative order of the selectivity index is fairly insensitive to when the measurement was taken; however, the weak trends observed in the selectivity index data likely reflect the relative rates of slowly reversible inhibition of the enzyme. Thus, it would appear that the slowly reversible reaction proceeds faster for cathepsins V and S than for cathepsin L; whereas, it proceeds more slowly for papain.

TABLE 14
IC₅₀ values of Compound No. 1 against papain and human cathepsins B, G, K, L, S, and V.

| | | | | | | % |
|---|---|---|---|---|---|---|
| | IC₅₀ @ 10 | IC₅₀ @ 30 | IC₅₀ @ 60 | IC₅₀ @ 90 | Selectivity | Identity |
| Enzyme | min (μM) | min (μM) | min (μM) | min (μM) | Index | to Cat L |
| Cathepsin L, | 0.155 ± 0.012 | 0.075 ± 0.005 | 0.056 ± 0.004 | 0.050 ± 0.003 | 1 | 100 |
| human liver | | | | | | |
| Cathepsin V, | 1.008 ± 0.1416 | 0.688 ± 0.039 | 0.618 ± 0.035 | 0.576 ± 0.024 | 7-11 | 78 |
| human, | | | | | | |
| recombinant, | | | | | | |
| NSO cells | | | | | | |
| Cathepsin S, | 1.107 ± 0.134 | 0.745 ± 0.010 | 0.724 ± 0.011 | 0.721 ± 0.029 | 7-14 | 55 |
| human spleen | | | | | | |
| Papain, Carica | 6.765 ± 0.367 | 2.981 ± 0.282 | 2.600 ± 0.208 | 1.562 ± 0.314 | 31-46 | 48 |
| papaya | | | | | | |
| Cathepsin B, | 7.492 ± 0.693 | 2.983 ± 0.295 | 2.527 ± 0.073 | 2.512 ± 0.139 | 40-50 | 26 |
| human liver | | | | | | |
| Cathepsin K, | 17.596 ± 1.040 | 8.460 ± 0.366 | 8.442 ± 0.140 | 6.857 ± 0.063 | 113-151 | 58 |
| human, | | | | | | |
| recombinant, | | | | | | |
| E. coli | | | | | | |
| Cathepsin G, | >50 | >50 | >50 | >50 | — | — |
| human | | | | | | |
| neutrophil | | | | | | |

### Biological Assays

Compound No. 1 was found to be non-toxic to human aortic endothelial

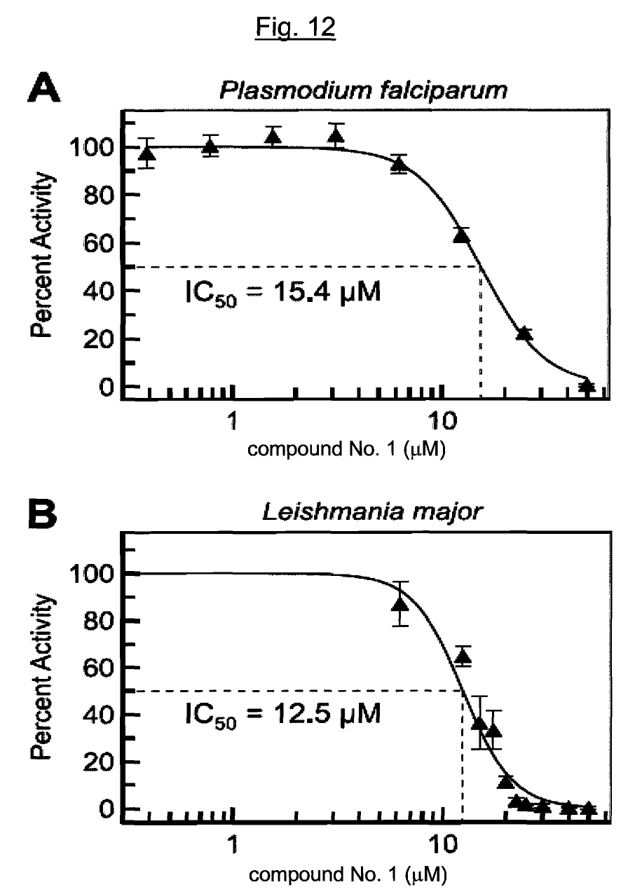

cells at 100 μM. The inhibitor also demonstrated a lack of toxicity to zebrafish in a live organism assay at 100 μM. Compound No. 1 was active in an in vitro propagation assay against Plasmodium falciparum with an IC₅₀ of 15.4±0.6 μM (FIG. 12A). Additionally, the thiocarbazate inhibitor was toxic toward Leishmania major promastigotes with an IC₅₀ of 12.5±0.6 μM (FIG. 12B).

TABLE 15
Selectivity indexes of Compound No. 1 against papain and human
cathepsins B, G, K, L, S, and V.

| | Selectivity | Selectivity | Selectivity | Selectivity |
|---|---|---|---|---|
| | Index @ | Index @ | Index @ | Index @ |
| Enzyme | 10 min | 30 min | 60 min | 90 min |
| Cathepsin L, human | 1 | 1 | 1 | 1 |
| liver | | | | |
| Cathepsin V, human, | 7 | 9 | 11 | 11 |
| recombinant, NSO cells | | | | |
| Cathepsin S, human | 7 | 10 | 13 | 14 |
| spleen | | | | |
| Papain, Carica papaya | 44 | 40 | 46 | 31 |
| Cathepsin B, human | 48 | 40 | 45 | 50 |
| liver | | | | |
| Cathepsin K, human, | 114 | 113 | 151 | 137 |
| recombinant, E. coli | | | | |
| Cathepsin G, human | — | — | — | — |
| neutrophil | | | | |

### Molecular Docking of Compound No. 1 in Papain

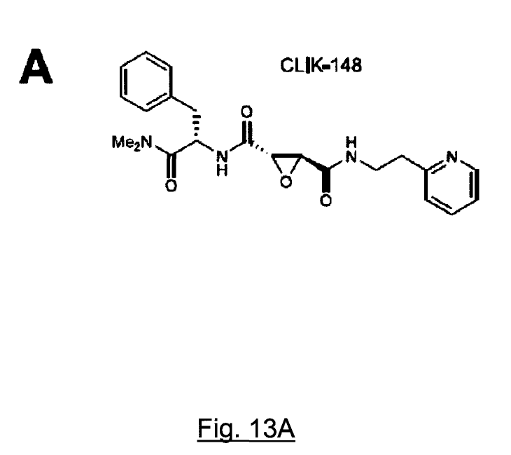

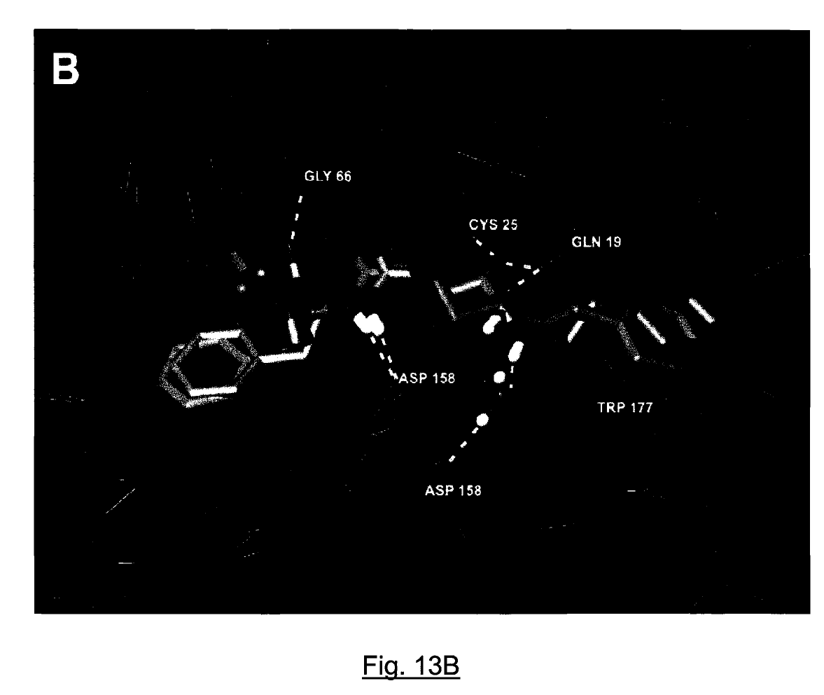

The co-crystal structure of CLIK-148 bound to papain (1 cvz.pdb) (Katunuma et al., 1999, FEBS Lett. 458:6-10; Tsuge et al., 1999, Biochem. Biophys. Res. Commun. 266:411-416) was used as a model to study hydrogen bonding and hydrophobic interactions of the thiocarbazate inhibitor Compound No. 1 within the cysteine protease binding site. The chemical structure of CLIK-148 is depicted in FIG. 13A. Other researchers have used papain to design highly specific cathepsin inhibitors and CLIK-148 directly inhibits cathepsin L (Katunuma et al., 1999, FEBS Lett. 458:6-10; LaLonde et al., 1998, J. Med. Chem. 41:4567-4576; Tsuge et al., 1999, Biochem. Biophys. Res. Commun. 266:411-416).

Molecular docking studies of CLIK-148 and Compound No. 1 in the binding site of papain were carried out using XP (extra precision) Glide software. Predictions of accurate binding modes have been accomplished with exceptional accuracy using XP Glide docking, resulting in computationally-derived protein/ligand complexes with adequate root mean square deviations from the known experimentally-derived co-crystal structure (Perola et al., 2004, Proteins 56:235-249). Initial docking studies were conducted on the papain/CLIK-148 system in order to verify that XP Glide could reproduce the binding mode of CLIK-148.

To prepare this system for docking, the covalent bond between CLIK-148 and papain was broken, and the epoxide ring-opened form of CLIK-148 was independently docked into papain. The highest scoring pose for CLIK-148 obtained from this docking study overlaid very well with the experimentally-derived bound inhibitor CLIK-148 (FIG. 13B). The XP glide score for CLIK-148 in papain was −9.27 kcal/mol. With this validation, the interaction of Compound No. 1 with papain was studied.

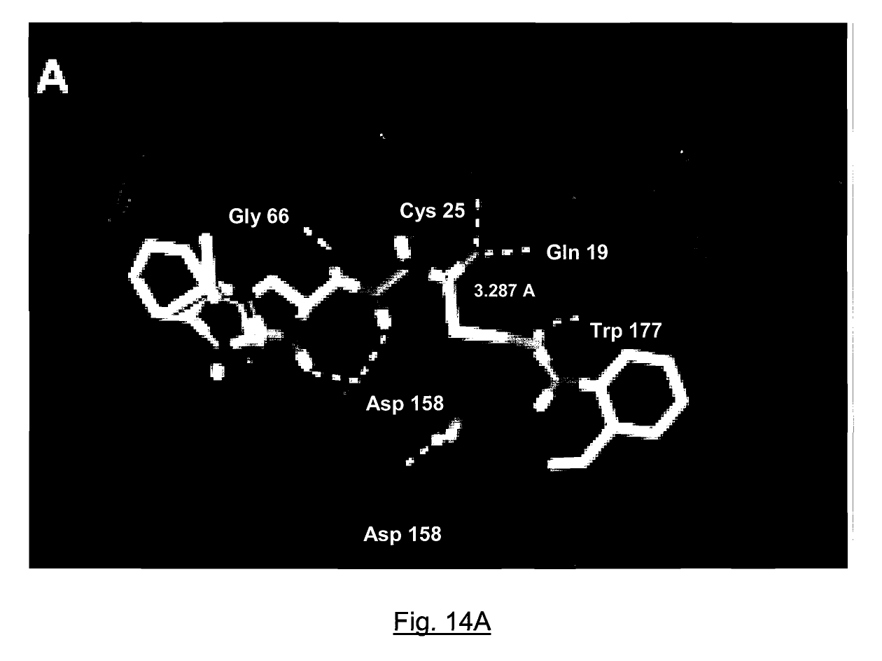

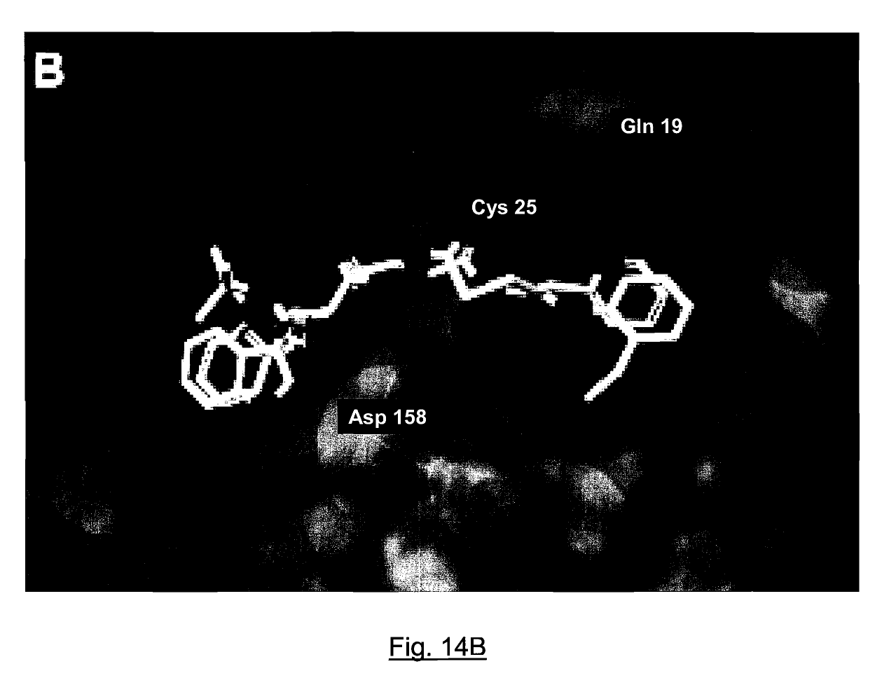

Compound No. 1 was prepared for docking using LigPrep software. The highest scoring pose of Compound No. 1 had an excellent score of −9.04 kcal/mol. This score was very close to the XP Glide score obtained for independently docked CLIK-148 in papain. In addition, many of the residues that made contacts between CLIK-148 and papain were also involved in making contacts between Compound No. 1 and papain (FIG. 14A). The backbone NH hydrogens of Gln19 and Cys25 made direct hydrogen bonding contacts to the thiocarbazate carbonyl oxygen of SID 26681509; the backbone NH hydrogen of Gly66 made a hydrogen bond to the acyl hydrazine CO oxygen of the ligand; the backbone carbonyl oxygen of Asp158 was involved in a hydrogen bonding network to both a hydrazine NH and an amide NH of Compound No. 1; and finally, the

Trp177 side chain NH formed a hydrogen bond to an amide carbonyl oxygen of Compound No. 1. In addition, the 2-ethylanilide group of Compound No. 1 made a large hydrophobic contact with the aromatic side chain of Trp177. Trp177 is located in the prime region of the enzyme binding pocket (S1′ subsite). The indole group of Compound No. 1 occupies the S2 subsite of the enzyme binding pocket. When the docking poses of CLIK-148 and Compound No. 1 were overlaid (FIG. 14B), Compound No. 1 looked remarkably like the epoxide ring-opened form of CLIK-148. This overlay illustrated that both inhibitors maintain the same critical distances between the two carbonyl groups that are disposed in a 1,4 relationship to each other. The intramolecular distance between the 1,4-dicarbonyl in both CLIK-148 and Compound No. 1 was approximately 4.80 to 4.92 Å.

Finally, the active site Cys25 sulfur is in close proximity (3.289 Å) to the carbonyl carbon of the thiocarbazate. Although the contribution from covalent bonding between this carbon and sulfur cannot be directly assessed through the docking studies presented herein, the molecule sits in the proper orientation to achieve this covalent binding interaction (FIG. 14A).

The kinetic analyses presented herein demonstrates that Compound No. 1 is a highly potent and selective competitive inhibitor of human cathepsin L with slow binding and slow reversibility kinetics. Molecular docking of Compound No. 1 into the papain crystal structure revealed hydrophobic interactions within the S2 and S1′ subsites and hydrogen bonding interactions with Gln19, Cys25, Gly66, Asp158, and Trp177. These interactions share a high degree of similarity with the papain/CLIK-148 complex (Katunuma et al., 1999, FEBS Lett. 458:6-10; LaLonde et al., 1998, J. Med. Chem. 41:4567-4576; Tsuge et al., 1999, Biochem. Biophys. Res. Commun. 266:411-416).

With the exception of cathepsin K, the range of selectivity indexes of Compound No. 1 decreased as the percent identity to cathepsin L increased. This may be due to the strong preference of cathepsin K for proline (Choe et al., 2006, J. Biol. Chem. 281:12824-12832). It is interesting to note that Compound No. 1 is 7 to 11 times more potent against cathepsin L than cathepsin V. Cathepsin V, which is sometimes referred to as cathepsin L2, shares 78% amino acid sequence identity with cathepsin L, and has been shown to compensate for the role of cathepsin L in epidermal homeostasis and hair follicle morphogenesis of knockout mice (Hagemann et al., 2004, Eur. J. Cell. Biol. 83:775-780; Nagler and Menard, 2003, Biol. Chem. 384:837-843; Reinheckel et al., 2001, Biol. Chem. 382:735-741).

Using the papain/CLIK-148 coordinate system, it was possible to independently dock Compound No. 1 into the binding site of papain. This led to the conclusion that Compound No. 1 appears to bind to papain in a manner similar to CLIK-148. Five residues that are conserved between papain and cathepsin L make direct contacts to both inhibitors. In addition, a highly hydrophobic/aromatic site involving Trp177 interacts with the hydrophobic 2-ethylanilide group of Compound No. 1.

The fact that Compound No. 1 inhibits both malaria and leishmaniasis suggests that it acts in a cellular system requiring transit across lipid membranes. However, the micromolar potency, as opposed to sub-nanomolar potency against purified human cathepsin L, was not surprising since i) there is as yet no measure of the internal concentration of inhibitor achieved in these organisms and ii) the active site geometries of their cathepsin L-like cysteine proteases might differ from that of the human enzyme. Further investigations of Compound No. 1 and related analogs against purified cathepsin L-like enzymes such as falcipain, congopain, cruzipain, T. gondii cathepsin L, histolysain, and rhodesain are warranted based on the findings of this study. The thiocarbazate scaffold can be readily derivatized, introducing functional groups to occupy specific binding sites in a variety of cysteine proteases, and thus holds promise as a general scaffold for the design of specific cysteine protease inhibitors.

### Example 6

### Identification and Synthesis of a Unique Oxycarbazite Cathepsin L Inhibitor

Previously reported inhibitors of cathepsin L include the peptides, leupeptin and aprotinin, and the fluoromethyl ketone, Z-LLL-FMK. The few known-potent small molecule inhibitors are either peptidic and therefore suffer from physiological instability and poor permeability, or are non-selective for cathepsin L (Esser et al., 1994, Arthritis Rheum. 37-236; Montaser et al., 2002, J. Biol. Chem. 383:1305; Fujishima et al., 1997, FEBS Lett. 407:47). The identification of potent, selective, stable, and cell permeable small-molecule inhibitors would therefore provide valuable tools to interrogate cathepsin L and cathepsin L-like function, as well as potential starting points for drug discovery and development.

Based on a previously disclosed thiocarbazate probe (Compound No. 1) depicted in FIG. 8, a new compound, Compound No. 96 depicted in FIG. 15, was designed by completing a ring structure connecting the ethyl group of the 2-ethylanilide to the nearby nitrogen atom. The resulting molecule is more hydrophobic, and thereby exhibits a higher binding affinity for the cathepsin L active site and more rapidly inhibits the cysteine protease.

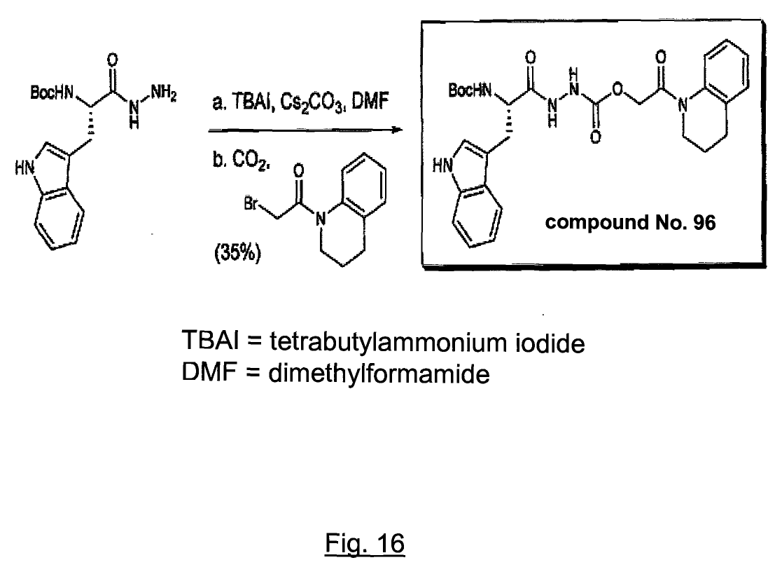

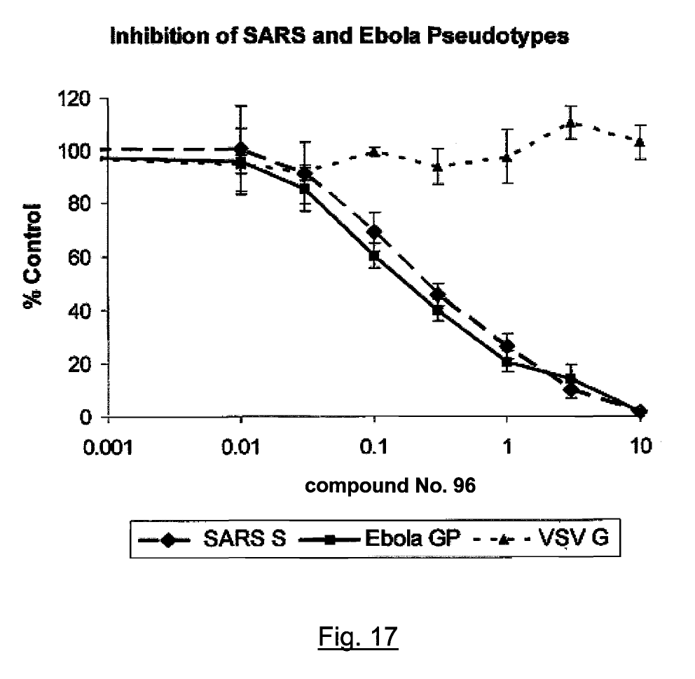

In addition, synthesis of analogs to this molecule replacing the sulfur atom of the molecule with carbon or oxygen revealed that oxygen is generally preferred in that position. This resulted in the development of Compound No. 96 (FIG. 15), an oxacarbazate cathepsin L inhibitor. The synthetic route for preparation of Compound No. 96 is shown in FIG. 16.

TABLE 16
Cathepsin L inhibitory activity of oxadiazole-containing library samples.^{a} 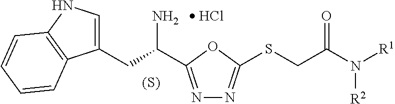
Impurities

| PubChem SID | R¹ | R² | IC50 (μM)^{b} |
|---|---|---|---|
| 861540 (1) | 2-ethylbenzene | H | 0.13 ± 0.01 |
| 861087 (2) | 2,4-dimethylbenzene | H | 0.16 ± 0.05 |
| 861840 (3) | Me | Me | 0.17 ± 0.02 |
| 861542 (4) | 2,3-dimethylbenzene | H | 0.30 ± 0.04 |
| 861992 (5) | Et | Et | 0.51 ± 0.02 |

^{a}Library samples contaning the parent oxadiazole with impurities.
^{b}IC₅₀ values are reported as mean ± standard deviation (number of determinations = 3).

TABLE 17
Cathepsin L inhibitory activity (IC₅₀ data, n = 3) of synthesized
compounds replacing the sulfur atom of Compound No. 1 with either
carbon or oxygen. Inhibitory data is also shown for the
tetrahydroquinoline compounds, replacing the same sulfur atom with
either carbon or oxygen. Compounds do not show inhibition against
cathepsin L when carbon is present in that position.

| | C | S | O |
|---|---|---|---|
| 2-ethylanilide | >50 μM | 0.056 ± 0.004 μM | 0.028 ± 0.004 μM |
| Tetrahydroquinoline | >50 μM | 0.041 ± 0.002 μM | 0.007 ± 0.001 μM |

An addition hydrogen bonding interaction between conserved His15A present in the binding site of papain is observed with the tetrahydroquinoline probe Compound No. 96 as compared to the thiocarbazate probe Compound No. 1. Accordingly, the docking score for tetrahydroquinoline Compound No. 96 in papain was correspondingly better at −10.00 kcal/mol as compared to −9.03 kcal/mol for thiocarbazate Compound No. 1 in papain.

### Example 7

### Specificity of Compound No. 96

SID 46493575 was tested for selectivity against human cathepsin B (Table 18) with no preincubation of enzyme and inhibitor. The selectivity ratio IC_{50,cat B}/IC_{50,cat L}) of SID 46493575 showed a 725-fold preference for inhibition of human cathepsin L over human cathepsin B.

TABLE 18
Specificity of Compound No. 96 for Cathepsin L.

| Enzyme | IC₅₀ (μM) | Selectivity Index |
|---|---|---|
| Cathepsin L, human liver | 0.007 ± 0.001 | 1.0 |
| Cathepsin B, human liver | 5.072 ± 0.883 | 724.6 |

### Example 8

### Efficacy of Tetrahydroquinoline Compound No. 96 in Treating Viral Infection

Severe acute respiratory syndrome coronavirus (SARS-CoV) and Ebola virus are hypothesized to function by trafficking to an intracellular compartment, wherein the contents of the viral package are released due to proteolytic cleavage by cathepsin L (Simmons et al., 2005, J. Virol. 79:12714-12720). The present invention is based upon the discovery that by inhibiting human cathepsin L using the tetrahydroquinoline SID 46493575, SARS-CoV and Ebola viral entry could be inhibited.

Compound No. 96 was tested in vitro in a SARS coronavirus pseudotype entry assay and an Ebola virus pseudotype entry assay using 293T cells. Compound No. 96 inhibits entry of both SARS-CoV (IC₅₀=273±49 nM) and Ebola virus (IC₅₀=193±39 nM). Vesicular stomatitis virus (VSV), which does not rely upon cathepsin L, was used as a control and has no effect (IC₅₀=10±10 μM).

A summary of probe properties of Compound No. 96 is shown in Table 19.

TABLE 19
Summary of properties of tetrahydroquinoline probe SID 46493575

| | |
|---|---|
| IC₅₀ (no preincubation) | 6.9 nM |
| IC₅₀ (4 hours preincubation) | 0.4 nM |
| (IC₅₀ Cat B)/(IC₅₀ Cat L) | 724.6 |
| Toxicity | Non-toxic to zebrafish at 100 μM |
| Viral entry assays | Inhibits SARS-CoV pseudotype infection |
| | with IC₅₀ ~200 nM. |
| | Inhibits Ebola virus pseudotype infection |
| | with IC₅₀ ~200 nM. |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising at least one compound of Formula I, or any pharmaceutically-acceptable salt thereof: 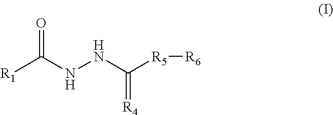
wherein:
R₁ is —CR_{2′}R_{2″}R₃ or heterocyclyl;
R_{2′} and R_{2″} are independently H, —NR₇R₈, —SR₇, acyl, aroyl, heteroaroyl, alkyl, substituted alkyl, aryl, substituted aryl, heteroaryl, substituted heteroaryl, heterocyclyl, or substituted heterocyclyl;
R₃ is H, —CHR₇R₈, alkyl, substituted alkyl, acyl, aroyl, heteroaroyl, aryl, substituted aryl, heteroaryl, substituted heteroaryl, heterocyclyl, substituted heterocyclyl —OR₇, or —SR₇;
R₄ is O or S;
R₅ is —O—, —S—, —C(═O)—, —NR₇— or a chemical bond;
R₆ is H, alkyl, substituted alkyl, aryl, substituted aryl, heteroaryl, substituted heteroaryl, heterocyclyl, or substituted heterocyclyl; and,
R₇ and R₈ are independently H, aroyl, heteroaroyl, alkyl, substituted alkyl, aryl, substituted aryl, heteroaryl, substituted heteroaryl, heterocyclyl, or substituted heterocyclyl.

2. The composition of claim 1, wherein:
(i) R₄ is O and R₅ is —S—;
(ii) R₄ is O and R₅ is —O—;
(iii) R₄ is O and R₅ is a chemical bond;
(iv) R₄ is O and R₅ is —NR₇; or
(v) R₄ is S and R₅ is —S—.

3-11. (canceled)

12. A pharmaceutical composition comprising a pharmaceutically acceptable carrier, and at least one compound selected from the group consisting of Compound No. 1, Compound No. 5, Compound No. 6, Compound No. 7, Compound No. 8, Compound No. 9, Compound No. 10, Compound No. 11, Compound No. 12, Compound No. 13, Compound No. 14, Compound No. 15, Compound No. 16, Compound No. 17, Compound No. 18, Compound No. 19, Compound No. 20, Compound No. 21, Compound No. 22, Compound No. 23, Compound No. 24, Compound No. 25, Compound No. 26, Compound No. 27, Compound No. 28, Compound No. 29, Compound No. 30, Compound No. 31, Compound No. 32, Compound No. 33, Compound No. 34, Compound No. 35, Compound No. 36, Compound No. 37, Compound No. 38, Compound No. 39, Compound No. 40, Compound No. 41, Compound No. 42, Compound No. 43, Compound No. 44, Compound No. 45, Compound No. 46, Compound No. 47, Compound No. 48, Compound No. 49, Compound No. 50, Compound No. 51, Compound No. 52, Compound No. 53, Compound No. 54, Compound No. 55, Compound No. 56, Compound No. 57, Compound No. 58, Compound No. 59, Compound No. 60, Compound No. 61, Compound No. 62, Compound No. 63, Compound No. 64, Compound No. 65, Compound No. 66, Compound No. 67, Compound No. 68, Compound No. 69, Compound No. 70, Compound No. 71, Compound No. 72, Compound No. 73, Compound No. 74, Compound No. 75, Compound No. 76, Compound No. 77, Compound No. 78, Compound No. 79, Compound No. 80, Compound No. 81, Compound No. 82, Compound No. 83, Compound No. 84, Compound No. 85, Compound No. 86, Compound No. 87, Compound No. 88, Compound No. 89, Compound No. 90, Compound No. 91, Compound No. 92, Compound No. 93, Compound No. 94, Compound No. 95, Compound No. 96, Compound No. 97, Compound No. 98, Compound No. 99, Compound No. 100, Compound No. 101, Compound No. 102, Compound No. 103, Compound No. 104, Compound No. 105, Compound No. 106, Compound No. 107, Compound No. 108, Compound No. 109, Compound No. 110, Compound No. 111, Compound No. 112, Compound No. 113, Compound No. 114, Compound No. 115, Compound No. 116, Compound No. 117, Compound No. 118, Compound No. 119, Compound No. 120, Compound No. 122, Compound No. 123, Compound No. 124, Compound No. 125, Compound No. 126, and Compound No. 127.

13-14. (canceled)

15. A method of inhibiting cathepsin L activity, said method comprising contacting a medium comprising cathepsin L with an effective amount of an inhibitor compound selected from the group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof, wherein when said inhibitor compound contacts said medium comprising cathepsin L, the activity of said cathepsin L is inhibited.

16. The method of claim 15, wherein:
(i) said thiocarbazate comprises a compound selected from the group consisting of Compound Nos. 1 and 5-92;
(ii) said oxacarbazate comprises a compound selected from the group consisting of Compound Nos. 93-112;
(iii) said diacyl hydrazine comprises a compound selected from the group of compounds consisting of Compound Nos. 113-117;
(iv) said acyl hydrazine comprises a compound selected from the group of compounds consisting of Compound Nos. 118-119;
(v) said N-hydroxy-amide comprises Compound No. 120;
(vi) said dialdehyde comprises Compound No. 121;
(vii) said sulfonylated acyl hydrazine comprises Compound No. 122;
(viii) said acyl hydrazone comprises a compound selected from the group of compounds consisting of Compound Nos. 123-124;
(ix) said acyl hydrazine carboxamide comprises Compound No. 125;
(x) said acyl hydrazine carbodithioate comprises Compound No. 126; or,
(xi) said acyl hydrazine oxoacetamide comprises Compound No. 127.

17-30. (canceled)

31. A method of inhibiting cathepsin B activity, said method comprising contacting a medium comprising cathepsin B with an effective amount of an inhibitor compound selected from the group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof, wherein when said inhibitor compound contacts said medium comprising cathepsin B, the activity of said cathepsin B is inhibited.

32. The method of claim 31, wherein:
(i) said thiocarbazate comprises a compound selected from the group consisting of Compound Nos. 1 and 5-92;
(ii) said oxacarbazate comprises a compound selected from the group consisting of Compound Nos. 93-112;
(iii) said diacyl hydrazine comprises a compound selected from the group of compounds consisting of Compound Nos. 113-117;
(iv) said acyl hydrazine comprises a compound selected from the group of compounds consisting of Compound Nos. 118-119;
(v) said N-hydroxy-amide comprises Compound No. 120;
(vi) said dialdehyde comprises Compound No. 121;
(vii) said sulfonylated acyl hydrazine comprises Compound No. 122;
(viii) said acyl hydrazone comprises a compound selected from the group of compounds consisting of Compound Nos. 123-124;
(ix) said acyl hydrazine carboxamide comprises Compound No. 125;
(x) said acyl hydrazine carbodithioate comprises Compound No. 126; or,
(xi) said acyl hydrazine oxoacetamide comprises Compound No. 127.

33-42. (canceled)

43. A method of inhibiting cathepsin S activity, said method comprising contacting a medium comprising cathepsin S with an effective amount of an inhibitor compound selected from the group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof, wherein when said inhibitor compound contacts said medium comprising cathepsin S, the activity of said cathepsin S is inhibited.

44. The method of claim 43, wherein:
(i) said thiocarbazate comprises a compound selected from the group consisting of Compound Nos. 1 and 5-92;
(ii) said oxacarbazate comprises a compound selected from the group consisting of Compound Nos. 93-112;
(iii) said diacyl hydrazine comprises a compound selected from the group of compounds consisting of Compound Nos. 113-117;
(iv) said acyl hydrazine comprises a compound selected from the group of compounds consisting of Compound Nos. 118-119;
(v) said N-hydroxy-amide comprises Compound No. 120;
(vi) said dialdehyde comprises Compound No. 121;
(vii) said sulfonylated acyl hydrazine comprises Compound No. 122;
(viii) said acyl hydrazone comprises a compound selected from the group of compounds consisting of Compound Nos. 123-124;
(ix) said acyl hydrazine carboxamide comprises Compound No. 125;
(x) said acyl hydrazine carbodithioate comprises Compound No. 126; or,
(xi) said acyl hydrazine oxoacetamide comprises Compound No. 127.

45-58. (canceled)

59. A method of treating a subject infected by or at risk of infection by, a viral pathogen, the method comprising administering a therapeutically effective amount of at least one cathepsin L inhibitor to the subject in need thereof, where the cathepsin L inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof.

60. The method of claim 59, wherein:
(i) said thiocarbazate comprises a compound selected from the group consisting of Compound Nos. 1 and 5-92;
(ii) said oxacarbazate comprises a compound selected from the group consisting of Compound Nos. 93-112;
(iii) said diacyl hydrazine comprises a compound selected from the group of compounds consisting of Compound Nos. 113-117;
(iv) said acyl hydrazine comprises a compound selected from the group of compounds consisting of Compound Nos. 118-119;
(v) said N-hydroxy-amide comprises Compound No. 120;
(vi) said dialdehyde comprises Compound No. 121;
(vii) said sulfonylated acyl hydrazine comprises Compound No. 122;
(viii) said acyl hydrazone comprises a compound selected from the group of compounds consisting of Compound Nos. 123-124;
(ix) said acyl hydrazine carboxamide comprises Compound No. 125;
(x) said acyl hydrazine carbodithioate comprises Compound No. 126; or,
(xi) said acyl hydrazine oxoacetamide comprises Compound No. 127.

61-71. (canceled)

72. The method of claim 59, wherein said infection is selected from the group consisting of SARS, Ebola, and Hendra virus.

73. A method of treating a subject infected by or at risk of infection by, a viral pathogen, the method comprising administering a therapeutically effective amount of at least one cathepsin B inhibitor to the subject in need thereof, wherein the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof.

74. The method of claim 73, wherein:
(i) said thiocarbazate comprises a compound selected from the group consisting of Compound Nos. 1 and 5-92;
(ii) said oxacarbazate comprises a compound selected from the group consisting of Compound Nos. 93-112;
(iii) said diacyl hydrazine comprises a compound selected from the group of compounds consisting of Compound Nos. 113-117;
(iv) said acyl hydrazine comprises a compound selected from the group of compounds consisting of Compound Nos. 118-119;
(v) said N-hydroxy-amide comprises Compound No. 120;
(vi) said dialdehyde comprises Compound No. 121;
(vii) said sulfonylated acyl hydrazine comprises Compound No. 122;
(viii) said acyl hydrazone comprises a compound selected from the group of compounds consisting of Compound Nos. 123-124;
(ix) said acyl hydrazine carboxamide comprises Compound No. 125;
(x) said acyl hydrazine carbodithioate comprises Compound No. 126; or,
(xi) said acyl hydrazine oxoacetamide comprises Compound No. 127.

75-83. (canceled)

84. The method of claim 73, wherein said infection is selected from the group consisting of SARS, Ebola, and Hendra virus.

85. A method of treating a subject afflicted with cancer, the method comprising administering a therapeutically effective amount of at least one cathepsin B inhibitor to the subject in need thereof, where the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof.

86. The method of claim 85, wherein:
(i) said thiocarbazate comprises a compound selected from the group consisting of Compound Nos. 1 and 5-92;
(ii) said oxacarbazate comprises a compound selected from the group consisting of Compound Nos. 93-112;
(iii) said diacyl hydrazine comprises a compound selected from the group of compounds consisting of Compound Nos. 113-117;
(iv) said acyl hydrazine comprises a compound selected from the group of compounds consisting of Compound Nos. 118-119;
(v) said N-hydroxy-amide comprises Compound No. 120;
(vi) said dialdehyde comprises Compound No. 121;
(vii) said sulfonylated acyl hydrazine comprises Compound No. 122;
(viii) said acyl hydrazone comprises a compound selected from the group of compounds consisting of Compound Nos. 123-124;
(ix) said acyl hydrazine carboxamide comprises Compound No. 125;
(x) said acyl hydrazine carbodithioate comprises Compound No. 126; or,
(xi) said acyl hydrazine oxoacetamide comprises Compound No. 127.

87-95. (canceled)

96. A method of treating a subject afflicted with or at risk of developing osteoporosis, the method comprising administering a therapeutically effective amount of at least one cathepsin B inhibitor to the subject in need thereof, where the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof.

97. The method of claim 96, wherein:
(i) said thiocarbazate comprises a compound selected from the group consisting of Compound Nos. 1 and 5-92;
(ii) said oxacarbazate comprises a compound selected from the group consisting of Compound Nos. 93-112;
(iii) said diacyl hydrazine comprises a compound selected from the group of compounds consisting of Compound Nos. 113-117;
(iv) said acyl hydrazine comprises a compound selected from the group of compounds consisting of Compound Nos. 118-119;
(v) said N-hydroxy-amide comprises Compound No. 120;
(vi) said dialdehyde comprises Compound No. 121;
(vii) said sulfonylated acyl hydrazine comprises Compound No. 122;
(viii) said acyl hydrazone comprises a compound selected from the group of compounds consisting of Compound Nos. 123-124;
(ix) said acyl hydrazine carboxamide comprises Compound No. 125;
(x) said acyl hydrazine carbodithioate comprises Compound No. 126; or,
(xi) said acyl hydrazine oxoacetamide comprises Compound No. 127.

98-106. (canceled)

107. A method of treating a subject afflicted with or at risk of developing arthritis, the method comprising administering a therapeutically effective amount of at least one cathepsin B inhibitor to the subject in need thereof, where the cathepsin B inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof.

108. The method of claim 107, wherein:
(i) said thiocarbazate comprises a compound selected from the group consisting of Compound Nos. 1 and 5-92;
(ii) said oxacarbazate comprises a compound selected from the group consisting of Compound Nos. 93-112;
(iii) said diacyl hydrazine comprises a compound selected from the group of compounds consisting of Compound Nos. 113-117;
(iv) said acyl hydrazine comprises a compound selected from the group of compounds consisting of Compound Nos. 118-119;
(v) said N-hydroxy-amide comprises Compound No. 120;
(vi) said dialdehyde comprises Compound No. 121;
(vii) said sulfonylated acyl hydrazine comprises Compound No. 122;
(viii) said acyl hydrazone comprises a compound selected from the group of compounds consisting of Compound Nos. 123-124;
(ix) said acyl hydrazine carboxamide comprises Compound No. 125;
(x) said acyl hydrazine carbodithioate comprises Compound No. 126; or,
(xi) said acyl hydrazine oxoacetamide comprises Compound No. 127.

109-117. (canceled)

118. A method of treating a subject infected by or at risk of infection by, a viral pathogen, the method comprising administering a therapeutically effective amount of at least one cathepsin S inhibitor to the subject in need thereof, where the cathepsin S inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof.

119. The method of claim 118, wherein:
(i) said thiocarbazate comprises a compound selected from the group consisting of Compound Nos. 1 and 5-92;
(ii) said oxacarbazate comprises a compound selected from the group consisting of Compound Nos. 93-112;
(iii) said diacyl hydrazine comprises a compound selected from the group of compounds consisting of Compound Nos. 113-117;
(iv) said acyl hydrazine comprises a compound selected from the group of compounds consisting of Compound Nos. 118-119;
(v) said N-hydroxy-amide comprises Compound No. 120;
(vi) said dialdehyde comprises Compound No. 121;
(vii) said sulfonylated acyl hydrazine comprises Compound No. 122;
(viii) said acyl hydrazone comprises a compound selected from the group of compounds consisting of Compound Nos. 123-124;
(ix) said acyl hydrazine carboxamide comprises Compound No. 125;
(x) said acyl hydrazine carbodithioate comprises Compound No. 126; or,
(xi) said acyl hydrazine oxoacetamide comprises Compound No. 127.

120-132. (canceled)

133. The method of claim 118, wherein said infection is selected from the group consisting of SARS, Ebola, and Hendra virus.

134. A method of treating a subject afflicted with hair loss, the method comprising administering a therapeutically effective amount of at least one cathepsin L inhibitor to the subject in need thereof, where the cathepsin L inhibitor is selected from a chemotype group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof.

135. The method of claim 134, wherein:
(i) said thiocarbazate comprises a compound selected from the group consisting of Compound Nos. 1 and 5-92;
(ii) said oxacarbazate comprises a compound selected from the group consisting of Compound Nos. 93-112;
(iii) said diacyl hydrazine comprises a compound selected from the group of compounds consisting of Compound Nos. 113-117;
(iv) said acyl hydrazine comprises a compound selected from the group of compounds consisting of Compound Nos. 118-119;
(v) said N-hydroxy-amide comprises Compound No. 120;
(vi) said dialdehyde comprises Compound No. 121;
(vii) said sulfonylated acyl hydrazine comprises Compound No. 122;
(viii) said acyl hydrazone comprises a compound selected from the group of compounds consisting of Compound Nos. 123-124;
(ix) said acyl hydrazine carboxamide comprises Compound No. 125;
(x) said acyl hydrazine carbodithioate comprises Compound No. 126; or,
(xi) said acyl hydrazine oxoacetamide comprises Compound No. 127.

136-146. (canceled)

147. A method of treating a subject afflicted with an autoimmune disease, said method comprising administering a therapeutically effective amount of at least one cathepsin S inhibitor to a subject in need thereof, wherein said cathepsin S inhibitor is selected from the group consisting of a thiocarbazate, oxacarbazate, a diacyl hydrazine, an acyl hydrazine, an N-hydroxy-amide, a dialdehyde, a sulfonylated acyl hydrazine, an acyl hydrazone, an acyl hydrazine carboxamide, an acyl hydrazine carbodithioate, an acyl hydrazine oxoacetamide, and a derivative, thereof.

148. The method of claim 147, wherein:
(i) said thiocarbazate comprises a compound selected from the group consisting of Compound Nos. 1 and 5-92;
(ii) said oxacarbazate comprises a compound selected from the group consisting of Compound Nos. 93-112;
(iii) said diacyl hydrazine comprises a compound selected from the group of compounds consisting of Compound Nos. 113-117;
(iv) said acyl hydrazine comprises a compound selected from the group of compounds consisting of Compound Nos. 118-119;
(v) said N-hydroxy-amide comprises Compound No. 120;
(vi) said dialdehyde comprises Compound No. 121;
(vii) said sulfonylated acyl hydrazine comprises Compound No. 122;
(viii) said acyl hydrazone comprises a compound selected from the group of compounds consisting of Compound Nos. 123-124;
(ix) said acyl hydrazine carboxamide comprises Compound No. 125;
(x) said acyl hydrazine carbodithioate comprises Compound No. 126; or,
(xi) said acyl hydrazine oxoacetamide comprises Compound No. 127.

149-161. (canceled)

162. The method of claim 147, wherein said autoimmune disease is selected from the group consisting of psoriasis, rheumatoid arthritis, multiple sclerosis, and asthma.
